SEQUENCE LISTING (18 sequences)

SEQ ID NO: 1 - protein, 22 aa, Artificial sequence
  Synthetic polypeptide
LLCFLVLTSLSHAFGQTDMSRK

SEQ ID NO: 2 - protein, 23 aa, Artificial sequence
  Synthetic polypeptide
INTIYLGGPFSPNVLNWRALKYE

SEQ ID NO: 3 - protein, 10 aa, Artificial sequence
  Synthetic polypeptide
GYSIFSYATK

SEQ ID NO: 4 - protein, 11 aa, Artificial sequence
  Synthetic polypeptide
KLISEEDLKAR

SEQ ID NO: 5 - protein, 224 aa, Artificial sequence
  Synthetic polypeptide
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 6 - protein, 6 aa, Artificial sequence
  Synthetic polypeptide
AFVFPK

SEQ ID NO: 7 - protein, 10 aa, Artificial sequence
  Synthetic polypeptide
ESDTSYVSLK

SEQ ID NO: 8 - protein, 12 aa, Artificial sequence
  Synthetic polypeptide
RQDNEILIFWSK

SEQ ID NO: 9 - protein, 10 aa, Artificial sequence
  Synthetic polypeptide
YEVQGEVFTK

SEQ ID NO: 10 - protein, 21 aa, Artificial sequence
  Synthetic polypeptide
GIVEQCCTSICSLYQLENYCN

SEQ ID NO: 11 - protein, 30 aa, Artificial sequence
  Synthetic polypeptide
FVNQHLCGSHLVEALYLVCGERGFFYTPKT

SEQ ID NO: 12 - protein, 17 aa, Artificial sequence
  Synthetic polypeptide
GIVEQCCTSICSLYQLE

SEQ ID NO: 13 - protein, 4 aa, Artificial sequence
  Synthetic polypeptide
NYCN

SEQ ID NO: 14 - protein, 9 aa, Artificial sequence
  Synthetic polypeptide
RGFFYTPKT

SEQ ID NO: 15 - protein, 4 aa, Artificial sequence
  Synthetic polypeptide
GIVE

SEQ ID NO: 16 - protein, 8 aa, Artificial sequence
  Synthetic polypeptide
ALYLVCGE

SEQ ID NO: 17 - protein, 13 aa, Artificial sequence
  Synthetic polypeptide
FVNQHLCGSHLVE

SEQ ID NO: 18 - protein, 13 aa, Artificial sequence
  Synthetic polypeptide
QCCTSICSLYQLE

(19) United States
(12) Patent Application Publication
(10) Pub. No.: US 2021/0379591 A1
(43) Pub. Date: Dec. 9, 2021

(54) FRAGMENTATION OF TARGET MOLECULES FOR SEQUENCING

(71) Applicant: Quantum-Si Incorporated, Guilford, CT (US)

(72) Inventors: Jonathan M. Rothberg, Guilford, CT (US); John H. Leamon, Stonington, CT (US); Jonathan C. Schultz, Guilford, CT (US); Michele Millham, Guilford, CT (US); Caixia Lv, Guilford, CT (US); Haidong Huang, Madison, CT (US); Roger Nani, Madison, CT (US); Omer Ad, Hamden, CT (US); Brian Reed, Madison, CT (US); Matthew Dyer, Spring, TX (US); Robert E. Boer, Westbrook, CT (US)

(73) Assignee: Quantum-Si Incorporated, Guilford, CT (US)

(21) Appl. No.: 17/236,826

(22) Filed: Apr. 21, 2021

Related U.S. Application Data
(60) Provisional application No. 63/014,093, filed on Apr. 22, 2020.
(60) Provisional application No. 63/139,346, filed on Jan. 20, 2021.

(51) Int. Cl. B01L 3/00; C12Q 1/6806

(52) U.S. Cl. CPC B01L 3/502738; B01L 3/502761; C12Q 1/6806; B01L 2200/027; B01L 2200/0668; B01L 2400/0644; B01L 2300/021; B01L 2300/041; B01L 2300/0816; B01L 2300/0883; B01L 2300/0887; B01L 2200/16

(57) ABSTRACT

Methods and devices for preparing target molecules (e.g., target nucleic acids or target proteins) from a biological sample are provided herein. In some embodiments, methods and devices involve sample lysis, sample fragmentation, enrichment of target molecule(s), and/or functionalization of target molecule(s).

### CROSS-REFERENCE TO RELATED APPLICATIONS

This application claims priority under 35 U.S.C. § 119(e) to U.S. Provisional Patent Applications 63/014,093, filed on Apr. 22, 2020, and 63/139,346, filed on Jan. 20, 2021; the entire contents of each of which are incorporated herein by reference.

### BACKGROUND OF INVENTION

Proteomics, genomics, and transcriptomics have emerged as important and necessary in the study of biological systems. These analysis of an individual organism or sample type can provide insights into cellular processes and response patterns, which lead to improved diagnostic and therapeutic strategies. The complexity surrounding nucleic acid and protein compositions and modification present challenges in determining large-scale sequencing information for a biological sample.

### SEQUENCE LISTING INCORPORATION BY REFERENCE

This application contains a Sequence Listing which has been filed electronically in ASCII and is hereby incorporated by reference in its entirety. This ASCII copy, created on Jul. 14, 2021, is named R070870097US02-SEQ-MSB and is 6,273 bytes in size.

### SUMMARY OF INVENTION

Aspects of the instant disclosure provide methods, compositions, devices, and/or cartridges for use in a process to prepare a sample for analysis and/or analyze (e.g., analyze by sequencing) one or more target molecules in a sample. In some embodiments, a target molecule is a nucleic acid (e.g., DNA or RNA, including without limitation, cDNA, genomic DNA, mRNA, and derivatives and fragments thereof). In some embodiments, a target molecule is a protein.

Some aspects of the disclosure provide devices for preparing a biological sample for sequencing. In some embodiments, the device comprises an automated module configured to receive two or more cartridges selected from the group consisting of (i) a lysis cartridge; (ii) an enrichment cartridge; (iii) a fragmentation cartridge; and (iv) a functionalization cartridge. In some embodiments, the device comprises an automated module comprising one or more microfluidic channels and configured to intake a biological sample comprising one or more target molecules. In some embodiments, the device comprises an automated module configured to receive (i) a lysis cartridge; and (ii) an enrichment cartridge. In some embodiments, the device comprises an automated module configured to receive (i) a lysis cartridge; and (iii) a fragmentation cartridge. In some embodiments, the device comprises an automated module configured to receive (i) a lysis cartridge; and (iv) a functionalization cartridge. In some embodiments, the device comprises an automated module configured to receive (ii) an enrichment cartridge; and (iii) a fragmentation cartridge. In some embodiments, the device comprises an automated module configured to receive (i) an enrichment cartridge; and (iv) a functionalization cartridge. In some embodiments, the device comprises an automated module configured to receive (i) a fragmentation cartridge; and (iv) a functionalization cartridge. In some embodiments, the device comprises an automated module configured to receive (i) a fragmentation cartridge; (ii) an enrichment cartridge; and (iii) a fragmentation cartridge. In some embodiments, the device comprises an automated module configured to receive (i) a fragmentation cartridge; (ii) an enrichment cartridge; and (iv) a functionalization cartridge. In some embodiments, the device comprises an automated module configured to receive (ii) an enrichment cartridge; (iii) a fragmentation cartridge; and (iv) a functionalization cartridge. In some embodiments, the device comprises an automated module configured to receive (i) a fragmentation cartridge; (ii) an enrichment cartridge; (iii) a fragmentation cartridge; and (iv) a functionalization cartridge. In some embodiments, the device produces nucleic acids with an average read-length that is longer than an average read-length produced using control methods. Further aspects of the disclosure provide devices for preparing one or more target molecules, configured to perform two or more of the following steps selected from (i), (ii), (iii), and (iv), wherein (i), (ii), (iii), and (iv) are defined as follows: (i) lyse a biological sample comprising one or more target molecules; (ii) enrich at least one of the one or more target molecules and/or at least one non-target molecule; (iii) fragment the one or more target molecules; and (iv) functionalize a terminal moiety of the one or more target molecules.

In some embodiments, one or more of the method steps selected from (i), (ii), (iii), and (iv) are performed in a cartridge. In some embodiments, the one or more steps are performed in the same cartridge. In some embodiments, the cartridge is a single-use cartridge or a multi-use cartridge. In some embodiments, the cartridge comprises one or more microfluidic channels configured to contain and/or transport a fluid used in any one of the automated steps. In some embodiments, the cartridge comprises one or more microfluidic channels configured to contain and/or transport the one or more target molecules between any one of the automated steps. In some embodiments, the cartridge comprises resin for purification of the one or more target molecules between any one of the automated steps. In some embodiments, the resin is Sephadex resin, optionally G-10 Sephadex resin. In some embodiments, the cartridge comprises any size exclusion medium.

Still further aspects of the disclosure provide methods for preparing one or more target molecules. In some embodiments, methods for preparing one or more target molecules comprise two or more of the following steps selected from (i), (ii), (iii), and (iv), wherein (i), (ii), (iii), and (iv) are defined as follows: (i) lyse a biological sample comprising one or more target molecules; (ii) enrich at least one of the one or more target molecules and/or at least non-target molecule; (iii) fragment the one or more target molecules; and (iv) functionalize a terminal moiety of the one or more fragmented target molecules; wherein at least one of steps (i), (ii), (iii), or (iv) is performed in an automated sample preparation device. In some embodiments, two steps are performed in an automated sample preparation device. In some embodiments, three steps are performed in an automated sample preparation device. In some embodiments, four steps are performed in an automated sample preparation device. In some embodiments, step (i) is performed using a lysis cartridge. In some embodiments, step (ii) is performed using an enrichment cartridge. In some embodiments, step (iii) is performed using a fragmentation cartridge. In some embodiments, step (iv) is performed using a functionalization cartridge.

Yet further aspects of the disclosure provide cartridges for preparing one or more target molecules. In some embodiments, a cartridge is configured to perform two or more of the following steps selected from (i), (ii), (iii), and (iv), wherein (ii), (iii), and (iv) are defined as follows: (i) lyse a biological sample comprising one or more target molecules; (ii) enrich at least one of the one or more target molecules and/or at least one non-target molecule; (iii) fragment the one or more target molecules; and (iv) functionalize a terminal moiety of the one or more target molecules. In some embodiments, the cartridge is a single-use cartridge or a multi-use cartridge. In some embodiments, the cartridge comprises one or more microfluidic channels configured to contain and/or transport a fluid used in any one of the automated steps. In some embodiments, the cartridge comprises one or more microfluidic channels configured to contain and/or transport the one or more target molecules between any one of the automated steps. In some embodiments, the cartridge comprises resin for purification of the one or more target molecules between any one of the automated steps. In some embodiments, the resin is Sephadex resin, optionally G-10 Sephadex resin.

In some embodiments, the biological sample is a single cell, mammalian cell tissue, animal sample, fungal sample, or plant sample. In some embodiments, the biological sample is a blood sample, saliva sample, sputum sample, fecal sample, urine sample, buccal swab sample, amniotic sample, seminal sample, synovial sample, spinal sample, or pleural fluid sample. In some embodiments, the one or more target molecules are nucleic acids. In some embodiments, the one or more target molecules are proteins.

In some embodiments, a device further comprises a peristaltic pump configured to transport one or more fluids into, within, or out of any one of cartridges received by the device. In some embodiments, a device further comprises a peristaltic pump configured to transport one or more fluids within, or through any of the microfluidic channels of cartridges received by the device. In some embodiments, a device is configured to transport fluids with a fluid flow resolution of less than or equal to 1000 microliters, less than or equal to 100 microliters, less than or equal to 50 microliters, or less than or equal to 10 microliters. In some embodiments, the device is configured to receive two or more cartridges at the same time. In some embodiments, the device is configured to establish fluidic communication between two or more cartridges received by the device at the same time. In some embodiments, the device is configured to receive two or more cartridges sequentially.

In some embodiments, the device further comprises a sequencing module. In some embodiments, the device is configured to deliver the one or more target molecules to the sequencing module. In some embodiments, the sequencing module performs nucleic acid sequencing. In some embodiments, the nucleic acid sequencing comprises single-molecule real-time sequencing, sequencing by synthesis, sequencing by ligation, nanopore sequencing, and/or Sanger sequencing. In some embodiments, the sequencing module performs protein sequencing. In some embodiments, the protein sequencing comprises Edman degradation or mass spectroscopy. In some embodiments, the sequencing module performs single-molecule protein sequencing.

In some embodiments, a lysis cartridge comprises one or more microfluidic channels and configured to intake a biological sample comprising one or more target molecules and produce a lysed sample. In some embodiments, an enrichment cartridge comprises one or more microfluidic channels and is configured to enrich at least one of the one or more target molecules to produce an enriched sample. In some embodiments, a fragmentation cartridge comprises one or more microfluidic channels and is configured to digest or fragment at least one of the one or more target molecules to produce a fragmented sample. In some embodiments, a functionalization cartridge comprises one or more microfluidic channels and is configured to functionalize a terminal moiety of at least one of the one or more target molecules to form a functionalized sample.

In some embodiments, any one cartridge is positioned to receive a sample or target molecule(s) from any other cartridge. In some embodiments, any one cartridge is connected by one or more microfluidic channels to any other cartridge.

In some embodiments, a lysis cartridge comprises reagents that lyse the sample but does not degrade or fragment the one or more target molecules. In some embodiments, the lysis cartridge comprises reagents that promote the one or more target molecules to be at least partially isolated or purified from non-target molecules of the sample. In some embodiments, the reagents comprise detergents, acids, and/or bases. In some embodiments, the reagents comprise a lysis buffer. In some embodiments, the lysis buffer is selected from the group consisting of: RIPA buffer, GCl (Guanidine-HCl) buffer, and GlyNP40 buffer. In some embodiments, the one or more microfluidic channels in the lysis cartridge promote shearing of cells and/or tissues (e.g., shear flow of cells and/or tissues). In some embodiments, the lysis cartridge comprises a needle passage that promotes mechanical shearing of cells and/or tissues. In some embodiments, the needle passage has an internal diameter of 0.1 to 1 mm. In some embodiments, the one or more microfluidic channels in the lysis cartridge comprise a post array. In some embodiments, the lysis cartridge is configured to be heated at an elevated temperature (e.g., 20-60° C., 20-30° C., 25-40° C., 30-50° C., 35-50° C., or 50-75° C.). In some embodiments, the device is configured to heat the lysis cartridge at an elevated temperature (e.g., 20-60° C., 20-30° C., 25-40° C., 30-50° C., 35-50° C., or 50-75° C.). In some embodiments, the device is configured to subject the lysis cartridge to microwaves or sonication.

In some embodiments, the enrichment cartridge comprises one or more affinity matrices. In some embodiments, the one or more affinity matrices are in microfluidic channels of the enrichment cartridge. In some embodiments, the one or more target molecules are nucleic acids, the immobilized capture probe is an oligonucleotide capture probe, and the oligonucleotide capture probe comprises a sequence that is at least partially complementary to at least one of the one or more target molecules. In some embodiments, the oligonucleotide capture probe comprises a sequence that is at least 80%, 90% 95%, or 100% complementary to the target molecule. In some embodiments, the one or more target molecules are proteins, and the immobilized capture probe is a protein capture probe that binds to at least one of the one or more target molecules. In some embodiments, the protein capture probe is an aptamer or an antibody. In some embodiments, the protein capture probe binds to the target protein with a binding affinity of 10-9 to 10-8 M, 10-8 to 10-7 M, 10-7 to 10-6 M, 10-6 to 10-5 M, 10-5 to 10-4 M, 10-4 to 10-3 M, or 10-3 to 10-2 M. In some embodiments, the one or more target molecules are nucleic acids, the immobilized capture probe is an oligonucleotide capture probe, and the oligonucleotide capture probe comprises a sequence that is at least partially complementary to at least one non-target molecule. In some embodiments, the oligonucleotide capture probe comprises a sequence that is at least 80%, 90% 95%, or 100% complementary to the non-target molecule. In some embodiments, the oligonucleotide capture probe is not complementary to the one or more target molecules. In some embodiments, the one or more target molecules are proteins, and the immobilized capture probe is a protein capture probe that binds to at least one non-target molecule. In some embodiments, the protein capture probe binds to the non-target protein with a binding affinity of 10-9 to 10-8 M, 10-8 to 10-7 M, 10-7 to 10-6 M, 10-6 to 10-5 M, 10-5 to 10-4 M, 10-4 to 10-3 M, or 10-3 to 10-2 M. In some embodiments, the protein capture probe does not bind to the one or more target molecules. In some embodiments, the enrichment cartridge is configured to deplete the sample of non-target molecules.

In some embodiments, the fragmentation cartridge comprises non-enzymatic reagents that digest or fragment the sample and/or the one or more target molecules. In some embodiments, the non-enzymatic reagents that digest or fragment the sample and/or the one or more target molecules comprise detergents, acids, and/or bases. In some embodiments, the non-enzymatic reagents that digest or fragment the sample and/or the one or more target molecules comprise cyanogen bromide, hydroxylamine, iodosobenzoic acid, dimethyl sulfoxide, hydrochloric acid, BNPS-skatole [2-(2-nitrophenylsulfenyl)-3-methylindole], and/or 2-nitro-5-thiocyanobenzoic acid. In some embodiments, the fragmentation cartridge comprises one or more enzymatic reagents that digest or fragment at least one of the one or more target molecules. In some embodiments, the one or more enzymatic reagents comprise one or more proteases. In some embodiments, the one or more proteases are selected from the group consisting of: trypsin, chymotrypsin, LysC, LysN, AspN, GluC and ArgC. In some embodiments, the one or more enzymatic reagents comprise one or more endonucleases or exonucleases. In some embodiments, the fragmentation cartridge can be heated at an elevated temperature (e.g., 20-60° C., 20-30° C., 25-40° C., 30-50° C., 35-50° C., or 50-75° C.). In some embodiments, a device is configured to heat the fragmentation cartridge at an elevated temperature (e.g., 20-60° C., 20-30° C., 25-40° C., 30-50° C., 35-50° C., or 50-75° C.). In some embodiments, a device is configured to subject the fragmentation cartridge to microwaves or sonication.

In some embodiments, the functionalization cartridge comprises a first chamber comprising reagents that covalently modify a moiety M0 of the one or more target molecules, or of one or more fragments thereof, to a modified moiety M1. In some embodiments, the reagents are non-enzymatic. In some embodiments, the covalent modification is regiospecific. In some embodiments, the portion of the one or more target molecules, or of the one or more fragments thereof, is a C-terminal carboxylate group or a C-terminal amino group. In some embodiments, the reagents comprise buffers, salts, organic compounds, acids, and/or bases. In some embodiments, the portion of the one or more target molecules, or of the one or more fragments thereof, is a C-terminal amino group, and the covalent modification is diazo transfer. In some embodiments, moiety M0 is —NH₂ and moiety M1 is —N₃. In some embodiments, the reagents comprise imidazole-1-sulfonyl azide and a copper salt (e.g., copper sulfate), and a buffer having a pH of about 9-11 (e.g. a potassium carbonate buffer having a pH of about 9-11). In some embodiments, the reagents comprise any azide transfer agent. In some embodiments, the reagents comprise trifluoromethanesulfonyl azide. In some embodiments, the azide transfer agent comprises benzenesulfonyl-azide. In some embodiments, the first chamber is connected via one or more microfluidic channels, and/or optionally a purification chamber, to a second chamber. In some embodiments, the second chamber comprises reagents that covalently modify moiety M1 to produce a functionalized peptide. In some embodiments, the covalent modification is an electrocyclic click reaction. In some embodiments, the reagents comprise a DBCO-labeled DNA-streptavidin conjugate and a buffer, optionally wherein the DBCO-labeled DNA-streptavidin conjugate is immobilized to the surface of the second chamber. In some embodiments, the functionalized peptide is functionalized with a DBCO-labeled DNA-streptavidin conjugate.

In some embodiments, a purification chamber is positioned between the first chamber and the second chamber, comprising a resin that promotes purification or enrichment of the modified target molecules, or fragments thereof. In some embodiments, the resin is Sephadex resin, optionally G-10 Sephadex resin. In some embodiments, the functionalization cartridge can be heated at an elevated temperature (e.g., 20-60° C., 20-30° C., 25-40° C., 30-50° C., 35-50° C., or 50-75° C.). In some embodiments, a device is configured to heat the functionalization cartridge at an elevated temperature (e.g., 20-60° C., 20-30° C., 25-40° C., 30-50° C., 35-50° C., or 50-75° C.). In some embodiments, the functionalization cartridge can be subjected to microwaves or sonication.

In some embodiments, purifying comprises passing the functionalized sample through a size exclusion medium. In some embodiments, the size exclusion medium may be a column. The column may be a desalting column. In some embodiments, the column is a Zeba column (e.g. a Zeba 7 kDa or a Zeba 40 kDa column). In some embodiments, the size exclusion medium is part of a fluidic device. In some embodiments, the size exclusion medium is part of a system, but is not part of a fluidic device of that system.

In some embodiments, purifying a protein comprises purification via immunoprecipitation. In some embodiments, immunoprecipitation comprises precipitating a target protein out of sample (e.g., a sample before or after functionalization) using an antibody that specifically binds to the target protein.

In some embodiments, the one or more microfluidic channels are configured to contain and/or transport fluid(s) and/or reagent(s).

In some embodiments, any one of the cartridges comprises a base layer having a surface comprising channels. In some embodiments, the channels include the one or more microfluidic channels. In some embodiments, at least a portion of at least some of the channels have a substantially triangularly-shaped cross-section having a single vertex at a base of the channel and having two other vertices at the surface of the base layer. In some embodiments, at least a portion of at least some of the channels of any one of the cartridges have a surface layer, comprising an elastomer, configured to substantially seal off a surface opening of the channel. In some embodiments, the elastomer comprises silicone. In some embodiments, at least one portion of at least some of the channels have walls and a base comprising a substantially rigid material compatible with biological material. In some embodiments, any one of the cartridges comprise one or more fluid reservoirs. In some embodiments, at least some of the channels connect to a reservoir in a temperature zone. In some embodiments, at least some of the channels connect to an electrophoresis gel.

### BRIEF DESCRIPTION OF DRAWINGS

FIG. 1 shows an example method for preparing a target molecule from a biological sample (e.g., using an automated sample preparation device or cartridge of the disclosure).

FIGS. 2A-2B show an example workflow for sample preparation of a target protein (e.g., using an automated sample preparation device or cartridge of the disclosure).

FIG. 3 shows an example workflow for sample lysis (e.g., using an automated device or cartridge of the disclosure).

FIG. 4 shows an example workflow for sample enrichment of a target molecule (e.g., using an automated device or cartridge of the disclosure).

FIG. 5 shows an example workflow for digestion of a target molecule (e.g., using an automated device or cartridge of the disclosure).

FIGS. 6-7 show example workflows for C-terminal functionalization of a target protein (e.g., using an automated device or cartridge of the disclosure).

FIG. 8 shows a schematic diagram of a cross-section view of a cartridge 100 along the width of channels 102, in accordance with some embodiments.

FIGS. 9A-9B show a top view schematic diagram (FIG. 9A) and an image of exemplary cartridges of the disclosure.

FIGS. 10A-10B show sequencing data output from DNA libraries generated with automated end-to-end (DNA extraction-to-finished library) sample preparation using a sample preparation device of the disclosure compared to libraries generated from manually extracted and purified DNA.

FIGS. 11A-11D show sequencing data output from a DNA library generated with automated end-to-end (DNA extraction-to-finished library) sample preparation using a sample preparation device of the disclosure compared to DNA libraries derived from samples that were size selected using commercial and manual methods.

FIG. 12 shows an example of a C-terminal carboxylate coupling procedure.

FIG. 13 shows an example of a C-terminal carboxylate coupling procedure.

FIGS. 14A-14D show examples of C-terminal coupling procedures. FIG. 14A shows representative functionalization of aspartic acid and glutamic acid terminated peptides. FIG. 14B shows representative functionalization of lysine and arginine terminated peptides. FIG. 14C shows an exemplary protection of sulfide moieties prior to functionalization of a lysine terminated peptide (Reaction 1), and an example of competitive intramolecular cyclization, which can be overcome using high concentrations of nucleophile and coupling reagent (Reaction 2). FIG. 14D shows model functionalization of a lysine terminated peptide (Reaction 3), and model functionalization of an arginine terminated peptide having internal glutamic acid and aspartic acid residues (Reaction 4).

FIG. 15 shows a model C-terminal lysine coupling procedure.

FIGS. 16A-16C show data related to a model C-terminal lysine coupling procedure. FIG. 16A and FIG. 16B show binding events to the N-terminus of QP126. The red arrow denotes when enzyme (peptidase) is added, after which a change in pulsing behavior is observed due to binding of the Clps to a different amino acid. FIG. 16C shows full length CRP sequence with bold fragments that were tagged).

FIG. 17 shows an example of a C-terminal lysine coupling procedure using the 4-nitrovinyl sulfonamide reagent.

FIGS. 18A-18B show schemes related to an exemplary C-terminal lysine coupling procedure using diazo transfer chemistry. FIG. 18A shows site-selective diazo transfer. FIG. 18B shows site-selective diazo transfer using a dipeptide followed by hydrolysis.

FIG. 19 shows an example of a lysine coupling procedure using diazo transfer.

FIG. 20 show representative schemes of solid-phase and solution-phase peptide activation methods.

FIG. 21 shows an example of a functionalization process using an immobilized carbodiimide reagent.

FIG. 22 shows an example of peptide surface immobilization.

FIGS. 23A-23B show representative examples of peptide sequencing. FIG. 23A shows a representative example of peptide sequencing by iterative cycles of terminal amino acid recognition and cleavage. FIG. 23B shows a representative example of dynamic peptide sequencing using a labeled amino acid recognition molecule and an exopeptidase in a single reaction mixture.

FIGS. 24A-24F show schematic diagrams of exemplary sample preparation devices of the disclosure.

FIGS. 25-26 show example workflows for C-terminal functionalization of a target protein (e.g., using an automated device or cartridge of the disclosure).

FIGS. 27A-27D show the results of sequencing peptide samples prepared in an exemplary fluidic device, according to certain embodiments.

### DETAILED DESCRIPTION OF INVENTION

### Sample Preparation Process

In some aspects, the disclosure provides processes for preparing a sample, e.g., for detection and/or analysis. In some embodiments, a process described herein may be used to identify properties or characteristics of a sample, including the identity or sequence (e.g., nucleotide sequence or amino acid sequence) of one or more target molecules in the sample. In some embodiments, a process may include one or more sample transformation steps, such as sample lysis, sample purification, sample fragmentation, purification of a fragmented sample, library preparation (e.g., nucleic acid library preparation), purification of a library preparation, sample enrichment (e.g., using affinity SCODA), and/or detection/analysis of a target molecule. In some embodiments, a sample may be a purified sample, a cell lysate, a single-cell, a population of cells, or a tissue. In some embodiments, a sample is any biological sample. In some embodiments, a sample (e.g., a biological sample) is a blood, saliva, sputum, feces, urine or buccal swab sample. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). A bacterial cell culture may comprise gram positive bacterial cells and/or gram-negative bacterial cells. In some embodiments, a sample is a purified sample of nucleic acids or proteins that have been previously extracted via user-developed methods from metagenomic samples or environmental samples. A blood sample may be a freshly drawn blood sample from a subject (e.g., a human subject) or a dried blood sample (e.g., preserved on solid media (e.g. Guthrie cards)). A blood sample may comprise whole blood, serum, plasma, red blood cells, and/or white blood cells.

In some embodiments, a sample (e.g., a sample comprising cells or tissue), may be prepared, e.g., lysed (e.g., disrupted, degraded and/or otherwise digested) in a process in accordance with the instant disclosure. In some embodiments, a sample to be prepared, e.g., lysed, comprises cultured cells, tissue samples from biopsies (e.g., tumor biopsies from a cancer patient, e.g., a human cancer patient), or any other clinical sample. In some embodiments, a sample comprising cells or tissue is lysed using any one of known physical or chemical methodologies to release a target molecule (e.g., a target nucleic acid or a target protein) from said cells or tissues. In some embodiments, a sample may be lysed using an electrolytic method, an enzymatic method, a detergent-based method, and/or mechanical homogenization. In some embodiments, a sample (e.g., complex tissues, gram positive or gram-negative bacteria) may require multiple lysis methods performed in series. In some embodiments, if a sample does not comprise cells or tissue (e.g., a sample comprising purified nucleic acids), a lysis step may be omitted. In some embodiments, lysis of a sample is performed to isolate target nucleic acid(s). In some embodiments, lysis of a sample is performed to isolate target protein(s). In some embodiments, a lysis method further includes use of a mill to grind a sample, sonication, surface acoustic waves (SAW), freeze-thaw cycles, heating, addition of detergents, addition of protein degradants (e.g., enzymes such as hydrolases or proteases), and/or addition of cell wall digesting enzymes (e.g., lysozyme or zymolase). Exemplary detergents (e.g., non-ionic detergents) for lysis include polyoxyethylene fatty alcohol ethers, polyoxyethylene alkylphenyl ethers, polyoxyethylene-polyoxypropylene block copolymers, polysorbates and alkylphenol ethoxylates, preferably nonylphenol ethoxylates, alkylglucosides and/or polyoxyethylene alkyl phenyl ethers. In some embodiments, lysis methods involve heating a sample for at least 1-30 min, 1-25 min, 5-25 min, 5-20 min, 10-30 min, 5-10 min, 10-20 min, or at least 5 min at a desired temperature (e.g., at least 60° C., at least 70° C., at least 80° C., at least 90° C., or at least 95° C.).

In some embodiments, a sample is prepared, e.g., lysed, in the presence of a buffer system. This buffer system may be used to make a slurry of the sample, to suspend the sample, and/or to stabilize the sample during any known lysis methodology, including those methods described herein. In some embodiments, a sample is prepared, e.g., lysed, in the presence of RIPA buffer, GCI buffer that comprises Guanidine-HCl buffer, Gly-NP40 buffer, a TRIS buffer, a HEPES buffer, or any other known buffering solution.

Many of the lysis methods described herein allow for the sample to be lysed by mechanically homogenizing the sample such that the cell walls of the sample break down. For example, methods that cause lysis by mechanical homogenization include, but are not limited to bead-beating, heating (e.g., to high temperatures sufficient to disrupt cell walls, e.g., greater than 50° C., 60° C., 70° C., 80° C., 90° C., or 95° C.), syringe/needle/microchannel passage (to cause shearing), sonication, or maceration with a grinder. In some embodiments, any lysis methodology may be combined with any other lysis methodology. For example, any lysis methodology may be combined with heating and/or sonication and/or syringe/needle/microchannel passage to quicken the rate of lysis.

In some embodiments, sample preparation comprises cell disruption (i.e., subsequent removal of unwanted cell and tissue elements following lysis). In some embodiments, cell disruption involves protein and/or nucleic acid precipitation. In some embodiments, following precipitation, the lysed and disrupted sample is subjected to centrifugation. In some embodiments, following centrifugation, the supernatant is discarded. Precipitation can be accomplished through multiple processes, including but not limited to those methods described in Winter, D. and H. Steen (2011). “Optimization of cell lysis and protein digestion protocols for the analysis of HeLa S3 cells by LC-MS/MS.” PROTEOMICS 11(24): 4726-4730. In some embodiments, proteins or peptides are immunoprecipitated. In some embodiments, centrifugation of precipitated proteins and/or nucleic acids is followed by discarding of the supernatant and subsequent washing of the pellet fraction (e.g., washing using chloroform/methanol or trichloroacetic acid).

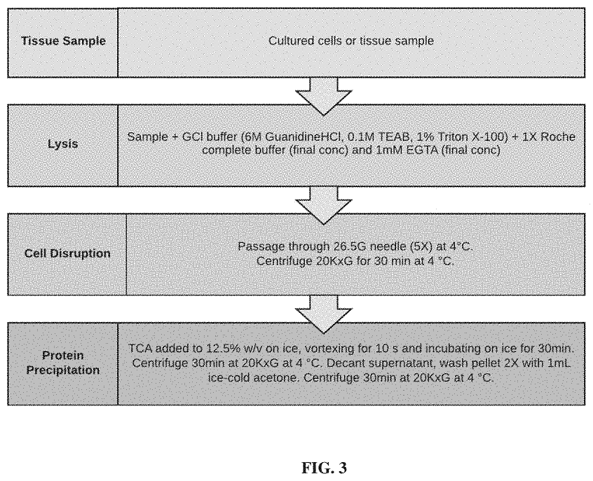

In some embodiments, a sample is prepared using lysis in the presence of a lysis buffer (e.g., GCI buffer (6M Guanidine HCl, 0.1 M TEAB, 1% Triton X-100, a standard buffer, and 1 mM EDTA/EGTA)) and disrupted by needle shearing (e.g., by passage of the sample through a 26.5 gauge needle, e.g., at 4° C.). In some embodiments, a lysed and disrupted sample is further subjected to precipitation of proteins and/or nucleic acids (e.g., using trichloroacetic acid at 4° C. with vortexing) and optionally followed by centrifugation. In some embodiments, a sample is prepared as described in FIG. 3.

In some embodiments, a sample (e.g., a sample comprising a target nucleic acid or a target protein) may be purified, e.g., following lysis, in a process in accordance with the instant disclosure. In some embodiments, a sample may be purified using chromatography (e.g., affinity chromatography that selectively binds the sample) or electrophoresis. In some embodiments, a sample may be purified in the presence of precipitating agents. In some embodiments, after a purification step or method, a sample may be washed and/or released from a purification matrix (e.g., affinity chromatography matrix) using an elution buffer. In some embodiments, a purification step or method may comprise the use of a reversibly switchable polymer, such as an electroactive polymer. In some embodiments, a sample may be purified by electrophoretic passage of a sample through a porous matrix (e.g., cellulose acetate, agarose, acrylamide).

In some embodiments, a sample (e.g., a sample comprising a target nucleic acid or a target protein) may be fragmented (i.e., digested) in a process in accordance with the instant disclosure. In some embodiments, a nucleic acid sample may be fragmented to produce small (<1 kilobase) fragments for sequence specific identification to large (up to 10+ kilobases) fragments for long read sequencing applications. Fragmentation of nucleic acids or proteins may, in some embodiments, be accomplished using mechanical (e.g., fluidic shearing), chemical (e.g., iron (Fe+) cleavage) and/or enzymatic (e.g., restriction enzymes, tagmentation using transposases) methods. In some embodiments, a protein sample may be fragmented to produce peptide fragments of any length. Fragmentation of proteins may, in some embodiments, be accomplished using chemical and/or enzymatic (e.g., proteolytic enzymes such as trypsin) methods. In some embodiments, mean fragment length may be controlled by reaction time, temperature, and concentration of sample and/or enzymes (e.g., restriction enzymes, transposases). In some embodiments, a nucleic acid may be fragmented by tagmentation such that the nucleic acid is simultaneously fragmented and labeled with a fluorescent molecule (e.g., a fluorophore). In some embodiments, a fragmented sample may be subjected to a round of purification (e.g., chromatography or electrophoresis) to remove small and/or undesired fragments as well as residual payload, chemicals and/or enzymes (e.g., transposases) used during the fragmentation step. For example, a fragmented sample (e.g., sample comprising nucleic acids) may be purified from an enzyme (e.g., a transposase), wherein the purification comprises denaturing the enzyme (e.g., by a combination of heat, chemical (e.g. SDS), and enzymatic (e.g. proteinase K) processes).

In some embodiments, the target molecule(s) is fragmented/digested prior to enrichment. In some embodiments, the target molecule is fragmented/digested after enrichment. In some embodiments, the target molecule(s) is fragmented/digested without any enrichment of the target molecule(s).

Fragmentation/digestion can be conducted using any known method, but typically will involve a non-enzymatic or enzymatic method. Non-enzymatic methods typically have an advantage as it relates to speed, simplicity, robustness, and ease of automation. These approaches include, but are not limited to, acid hydrolysis and/or cleavage using a chemical entity such as cyanogen bromide, hydroxylamine, iodosobenzoic acid, dimethyl sulfoxide-hydrochloric acid, BNPS-skatole [2-(2-nitrophenylsulfenyl)-3-methylindole], or 2-nitro-5-thiocyanobenzoic acid. Non-enzymatic, electro-physical digestion methods have been employed as well, including electrochemical oxidation and/or digestion in conjunction with microwaves. Enzymatic methods typically utilize proteases to fragment protein into component peptides. These enzymes include trypsin (which is typically favored for the size of the peptides generated and the generation of a basic residue at the carboxyl terminus of the peptide), chymotrypsin, LysC, LysN, AspN, GluC and/or ArgC.

Enzymatic fragmentation/digestion methods may be optimized for ease of use, speed, automation and/or effectiveness. In some embodiments, enzymatic methods include enzyme immobilization on solid substrates. In some embodiments, enzymatic methods are performed in flow (e.g., in a microfluidic channel).

Fragmentation/digestion methods may be performed using an automated device or module. Alternatively, or in addition, fragmentation/digestion methods may be performed manually. An enzymatic digestion may utilize any number or combination of enzymes and may further comprise any of the known non-enzymatic methods.

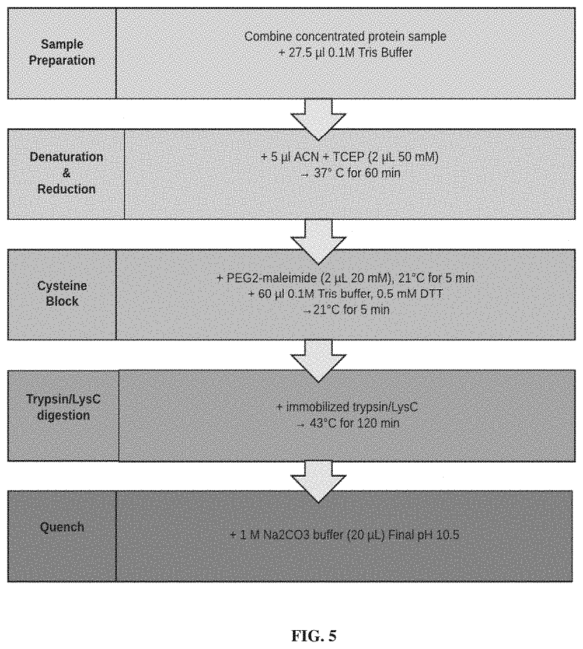

In some embodiments, a fragmentation/digestion process is as described in FIG. 5. In some embodiments, a sample comprising target protein(s) is first denatured and reduced (e.g., using acetonitrile and TCEP). In some embodiments, target protein(s) to be fragmented are subjected to capping of an amino acid side chain (e.g., a cysteine block) (e.g., using an amino acid side chain capping agent). In some embodiments, target protein(s) are fragmented using a mixture of trypsin and LysC (e.g., for 120 minutes). Enzymatic reactions may be quenched (e.g., using sodium carbonate buffer).

Any suitable reducing agent may be used to reduce a target protein within a sample. In some embodiments, the reducing agent is suitable for reducing a disulfide-bond. In some embodiments, the reducing agent may reversibly reduce a disulfide bond. Suitable reversable reducing agents may comprise compounds such as dithiothreitol (DTT), β-mercaptoethanol (BME), and/or Glutathione (GSH). In some embodiments, the reducing agent may irreversibly reduce a disulfide bond. Suitable irreversible reducing agents may comprise compounds such as tris(2-carboxyethyl)phosphine (TCEP). In some specific embodiments, the reducing agent comprises tris(2-carboxyethyl)phosphine (TCEP).

Any suitable amino acid side chain capping agent may be used to cap amino acid side chains of a protein within a peptide sample. In some embodiments, the amino acid side chain capping agent prevents the formation of disulfide bonds. In some embodiments, the amino acid side chain capping agent prevents the amino acid side chain from undergoing further reactivity such as nucleophile/electrophile or redox reactivity. In some embodiments, the amino acid side chain capping agent is a cysteine capping agent. In some embodiments, the amino acid side chain capping agent is a sulfhydryl-reactive alkylating reagent (e.g. a cysteine alkylation agent). For instance, in some embodiments, the amino acid side chain capping agent comprises a haloacetamide (e.g. chloroacetamide, iodoacetamide) or a haloacetate/haloacetic acid (e.g., chloroacetate/chloroacetic acid, iodoacetate/iodoacetic acid). In some embodiments, the amino acid side chain capping agent is an aromatic benzyl halide. Other examples of suitable cysteine alkylating agents include 4-vinylpyridine, acrylamide, and methanethiosulfonate, In some embodiments, the amino acid side chain capping agent comprises iodoacetamide.

In some embodiments, a sample comprising a target nucleic acid may be used to generate a nucleic acid library for subsequent analysis (e.g., genomic sequencing) in a process in accordance with the instant disclosure. A nucleic acid library may be a linear library or a circular library. In some embodiments, nucleic acids of a circular library may comprise elements that allow for downstream linearization (e.g., endonuclease restriction sites, incorporation of uracil). In some embodiments, a nucleic acid library may be purified (e.g., using chromatography, e.g., affinity chromatography), or electrophoresis.

In some embodiments, a library of nucleic acids (e.g., linear nucleic acids) is prepared using end-repair, a process wherein a combination of enzymes (e.g., Taq DNA Ligase, Endonuclease IV, Bst DNA Polymerase, Fpg, Uracil-DNA Glycosylase, T4 Endonuclease V and/or Endonuclease VIII) extend the 3′ end of the nucleic acids, generating a complement to the 5′ payload, and repairing any abasic sites or nicks in the nucleic acids. In some embodiments, a library of linear nucleic acids is prepared using a self-priming hairpin adaptor, a process which may obviate the need to anneal a unique sequencing primer to an individual nucleic acid fragment primer prior to formation of a polymerase complex. Following end-repair, a library of nucleic acids (e.g., linear nucleic acids) may be purified using solid-phase adsorption with subsequent elution into a fresh buffer, using passage of the nucleic acids through a size-selective matrix (e.g., agarose gel). The size-selective matrix may be used to remove nucleic acid fragments that are smaller than the size of the target nucleic acids.

In some embodiments, a sample (e.g., a sample comprising a target nucleic acid or a target protein) may be enriched for a target molecule in a process in accordance with the instant disclosure. Enrichment is typically used when the complexity of the un-enriched sample exceeds the capacity of the sequencing platform, or when the target molecule is present in the sample at a low abundance (e.g., such that it cannot be easily detected by the sequencing platform). Enrichment involves the use of a mechanism that selectively amplifies the target molecule. This enrichment may involve the use of antibodies, aptamers, size-based selection, or electrostatic charge-based selection in order to selectively amplify the target molecule(s) (e.g., target protein(s) or target nucleic acid(s)).

Enrichment may typically be used when the intent of the sample preparation is to sequence specific target molecules. Enrichment may be used to perform or conduct a proteomic, genomic, or metagenomic analysis or survey, when the target molecules are related or homologous to one another.

In some embodiments, a sample is enriched for a target molecule using an electrophoretic method. In some embodiments, a sample is enriched for a target molecule using affinity SCODA. In some embodiments, a sample is enriched for a target molecule using field inversion gel electrophoresis (FIGE). In some embodiments, a sample is enriched for a target molecule using pulsed field gel electrophoresis (PFGE). In some embodiments, the matrix used during enrichment (e.g., a porous media, electrophoretic polymer gel) comprises immobilized affinity agents (also known as ‘immobilized capture probes’) that bind to target molecule present in the sample. In some embodiments, a matrix used during enrichment comprises 1, 2, 3, 4, 5, or more unique immobilized capture probes, each of which binds to a unique target molecule and/or bind to the same target molecule with different binding affinities.

In some embodiments, an immobilized capture probe is an oligonucleotide capture probe that hybridizes to a target nucleic acid. In some embodiments, an oligonucleotide capture probe is at least 50%, 60%, 70%, 80%, 90% 95%, or 100% complementary to a target nucleic acid. In some embodiments, a single oligonucleotide capture probe may be used to enrich a plurality of related target nucleic acids (e.g., 2, 3, 4, 5, 6, 7, 8, 9, 10, 20, 30, 40, 50, or more related target nucleic acids) that share at least 50%, 60%, 70%, 80%, 90% 95%, or 99% sequence identity. Enrichment of a plurality of related target nucleic acids may allow for the generation of a metagenomic library. In some embodiments, an oligonucleotide capture probe may enable differential enrichment of related target nucleic acids. In some embodiments, an oligonucleotide capture probe may enable enrichment of a target nucleic acid relative to a nucleic acid of identical sequence that differs in its modification state (e.g., single nucleotide polymorphism, methylation state, acetylation state). In some embodiments, an oligonucleotide capture probe is used to enrich human genomic DNA for a specific gene of interest (e.g., HLA). A specific gene of interest may be a gene that is relevant to a specific disease state or disorder. In some embodiments, an oligonucleotide capture probe is used to enrich nucleic acid(s) of a metagenomic sample.

In some embodiments, for the purposes of enriching nucleic acid target molecules with a length of 0.5-2 kilobases, oligonucleotide capture probes may be covalently immobilized in an acrylamide matrix using a 5′ Acrydite moiety. In some embodiments, for the purposes of enriching larger nucleic acid target molecules (e.g., with a length of >2 kilobases), oligonucleotide capture probes may be immobilized in an agarose matrix. In some embodiments, oligonucleotide capture probes may be immobilized in an agarose matrix using thiol-epoxide chemistries (e.g., by covalently attached thiol-modified oligonucleotides to crosslinked agarose beads). Oligonucleotide capture probes linked to agarose beads can be combined and solidified within standard agarose matrices (e.g., at the same agarose percentage).

In some embodiments, enrichment of nucleic acids using methods described herein (e.g., enrichment using SCODA) produces nucleic acid target molecules that comprise a length of about 0.5 kilobases (kb), about 1 kb, about 1.5 kb, about 2 kb, about 3 kb, about 4 kb, about 5 kb, about 6 kb, about 7 kb, about 8 kb, about 9 kb, about 10 kb, about 12 kb, about 15 kb, about 20 kb, or more. In some embodiments, enrichment of nucleic acids using methods described herein (e.g., enrichment using SCODA) produces nucleic acid target molecules that comprise a length of about 0.5-2 kb, 0.5-5 kb, 1-2 kb, 1-3 kb, 1-4 kb, 1-5 kb, 1-10 kb, 2-10 kb, 2-5 kb, 5-10 kb, 5-15 kb, 5-20 kb, 5-25 kb, 10-15 kb, 10-20 kb, or 10-25 kb.

In some embodiments, an immobilized capture probe is a protein capture probe (e.g., an aptamer or an antibody) that binds to a target protein or peptide fragment. In some embodiments, a protein capture probe binds to a target protein or peptide fragment with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M. In some embodiments, the binding affinity is in the picomolar to nanomolar range (e.g., between about 10⁻¹² and about 10⁻⁹ M). In some embodiments, the binding affinity is in the nanomolar to micromolar range (e.g., between about 10⁻⁹ and about 10⁻⁶ M). In some embodiments, the binding affinity is in the micromolar to millimolar range (e.g., between about 10⁻⁶ and about 10⁻³ M). In some embodiments, the binding affinity is in the picomolar to micromolar range (e.g., between about 10⁻¹² and about 10⁻⁶ M). In some embodiments, the binding affinity is in the nanomolar to millimolar range (e.g., between about 10⁻⁹ and about 10⁻³ M). In some embodiments, a single protein capture probe may be used to enrich a plurality of related target proteins that share at least 50%, 60%, 70%, 80%, 90% 95%, or 99% sequence identity. In some embodiments, a single protein capture probe may be used to enrich a plurality of related target proteins (e.g., 2, 3, 4, 5, 6, 7, 8, 9, 10, 20, 30, 40, 50, or more related target proteins) that share at least 50%, 60%, 70%, 80%, 90% 95%, or 99% sequence homology. Enrichment of a plurality of related target proteins may allow for the generation of a metaproteomics library. In some embodiments, a protein capture probe may enable differential enrichment of related target proteins.

In some embodiments, multiple capture probes (e.g., populations of multiple capture probe types, e.g., that bind to deterministic target molecules of infectious agents such as adenovirus, staphylococcus, pneumonia, or tuberculosis) may be immobilized in an enrichment matrix. Application of a sample to an enrichment matrix with multiple deterministic capture probes may result in diagnosis of a disease or condition (e.g., presence of an infectious agent). In some embodiments, a target molecule or related target molecules may be released from the enrichment matrix after removal of non-target molecules, in a process in accordance with the instant disclosure. In some embodiments, a target molecule may be released from the enrichment matrix by increasing the temperature of the enrichment matrix. Adjusting the temperature of the matrix further influences migration rate as increased temperatures provide a higher capture probe stringency, requiring greater binding affinities between the target molecule and the capture probe. In some embodiments, when enriching related target molecules, the matrix temperature may be gradually increased in a step-wise manner in order to release and isolate target molecules in steps of ever-increasing homology. In some embodiments, temperature is increased by about 5%, 10%, 15%, 20%, 25%, 30%, 40%, 50%, or more in each step or over a period of time (e.g., 1-10 min, 1-5 min, or 4-8 min). In some embodiments, temperature is increased by 5%-10%, 5-15%, 5%-20%, 5%-25%, 5%-30%, 5%-40%, 5%-50%, 10%-25%, 20%-30%, 30%-40%, 35%-50%, or 40%-70% in each step or over a period of time (e.g., 1-10 min, 1-5 min, or 4-8 min). In some embodiments, temperature is increased by about 1° C., 2° C., 3° C., 4° C., 5° C., 6° C., 7° C., 8° C., 9° C., or 10° C. in each step or over a period of time (e.g., 1-10 min, 1-5 min, or 4-8 min). In some embodiments, temperature is increased by 1-10° C., 1-5° C., 2-5° C., 2-10° C., 3-8° C., 4-9° C., or 5-10° C. in each step or over a period of time (e.g., 1-10 min, 1-5 min, or 4-8 min). This may allow for the sequencing of target proteins or target nucleic acids that are increasingly distant in their relation to an initial reference target molecule, enabling discovery of novel proteins (e.g., enzymes) or functions (e.g., enzymatic function or gene function). In some embodiments, when using multiple capture probes (e.g., multiple deterministic capture probes), the matrix temperature may be increased in a step-wise or gradient fashion, permitting temperature-dependent release of different target molecules and resulting in generation of a series of barcoded release bands that represent the presence or absence of control and target molecules.

Enrichment of a sample (e.g., a sample comprising a target nucleic acid or a target protein) allows for a reduction in the total volume of the sample. For example, in some embodiments, the total volume of a sample is reduced after enrichment by at least 10%, at least 20%, at least 30%, at least 40%, at least 50%, at least 60%, at least 70%, at least 80%, at least 90%, at least 100%, or at least 120%. In some embodiments, the total volume of a sample is reduced after enrichment from 1-20 mL initial volume to 100-1000 μL final volume, from 1-5 mL initial volume to 100-1000 μL final volume, from 100-1000 μL initial volume to 25-100 μL final volume, from 100-500 μL initial volume to 10-100 μL final volume, or from 50-200 μL initial volume to 1-25 μL final volume. For example, in some embodiments, the final volume of a sample after enrichment is 10-100 μL, 10-50 μL, 10-25 μL, 20-100 μL, 20-50 μL, 25-100 μL, 25-250 μL, 25-1000 μL, 100-1000 μL, 100-500 μL, 100-250 μL, 200-1000 μL, 200-500 μL, 200-750 μL, 500-1000 μL, 500-1500 μL, 500-750 μL, 1-5 mL, 1-10 mL, 1-2 mL, 1-3 mL, or 1-4 mL.

In addition to amplification of the target molecule, or as an alternative to amplification of the target molecule, a sample may be enriched (e.g., for a low abundance target molecule) by depletion of unwanted non-target molecules (e.g., high-abundance proteins (e.g. albumin)). Depletion of unwanted non-target molecules may be performed using similar capture strategies as discussed above. When using a depletion strategy, the capture probes will bind to unwanted, non-target molecules and allow for target molecules to remain in solution. This strategy equally enables enrichment of the target molecule (i.e., increased relative concentrations of the target molecule(s)).

For example, an immobilized capture probe that is used for depletion may be an oligonucleotide capture probe that hybridizes to an unwanted non-target nucleic acid. In some embodiments, an oligonucleotide capture probe that is used for depletion is at least 50%, 60%, 70%, 80%, 90% 95%, or 100% complementary to an unwanted non-target nucleic acid. In some embodiments, a single oligonucleotide capture probe that is used for depletion may be used to deplete a plurality of related target nucleic acids (e.g., 2, 3, 4, 5, 6, 7, 8, 9, 10, 20, 30, 40, 50, or more related target nucleic acids) that share at least 50%, 60%, 70%, 80%, 90% 95%, or 99% sequence identity.

In some embodiments, an immobilized capture probe that is used for depletion is a protein capture probe (e.g., an aptamer or an antibody) that binds to an unwanted non-target protein or peptide fragment. In some embodiments, a protein capture probe that is used for depletion binds to an unwanted non-target protein or peptide fragment with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M. In some embodiments, the binding affinity is in the nanomolar to millimolar range (e.g., between about 10⁻⁹ and about 10⁻³ M). In some embodiments, a single protein capture probe that is used for depletion may be used to deplete a plurality of related target proteins that share at least 50%, 60%, 70%, 80%, 90% 95%, or 99% sequence identity. In some embodiments, a single protein capture probe that is used for depletion may be used to deplete a plurality of related target proteins (e.g., 2, 3, 4, 5, 6, 7, 8, 9, 10, 20, 30, 40, 50, or more related target proteins) that share at least 50%, 60%, 70%, 80%, 90% 95%, or 99% sequence homology. In some embodiments, enrichment comprises amplification of target molecule(s) and depletion (e.g., of high abundance proteins). In some embodiments, depletion steps are performed before amplification and enrichment of target molecule(s). In some embodiments, in order to avoid possible contamination of the target molecule(s) by the capture elements of the enrichment process (e.g., antibodies or aptamers), the capture elements are depleted from an enriched sample (i.e., after enrichment by either amplification of target molecules and/or depletion of unwanted non-target molecules from the original sample).

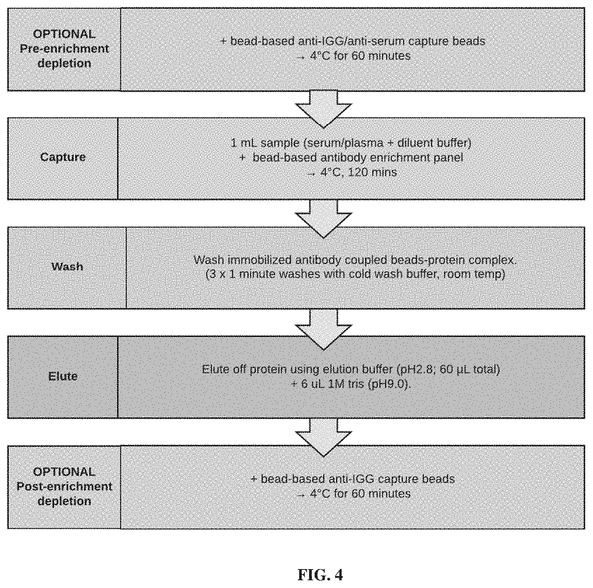

In some embodiments, a sample is first subjected to a depletion step (e.g., to remove unwanted non-target proteins). In some embodiments, a sample is enriched using amplification or immobilized target capture (e.g., using antibodies to selectively enrich for a target protein) following a first depletion step. Following amplification or immobilized target capture, the sample may then be subjected to a second depletion step (e.g., to remove excess antibody or capture probe). In some embodiments, a sample is enriched, for example, as described in FIG. 4.

In some embodiments, any number of enrichment steps (e.g., amplification of target molecule(s) and/or depletion(s)) can be performed by the automated device or module (e.g., on a chip or cartridge). In some embodiments, the enrichment steps are amenable to automation on the cartridge using capture elements (e.g., antibodies) immobilized on solid phase structures.

In some embodiments, any immobilized capture element or probe described herein may be on any solid support structure or surface. The solid support structure or surface may be magnetic and/or may be a frit, a filter, a chip, or a cartridge surface. In some embodiments, the capture elements or probes for enrichment may be interchanged (e.g., using flow on a chip).
In some embodiments, any number of the enrichment steps are performed manually. If performed manually, any enriched target molecule may be subsequently placed into an automated sample preparation device described herein.

In some embodiments, a target molecule or target molecules may be detected after enrichment and subsequent release to enable analysis of said target molecule(s) and its upstream sample, in a process in accordance with the instant disclosure. In some embodiments, a target nucleic acid may be detected using gene sequencing, absorbance, fluorescence, electrical conductivity, capacitance, surface plasmon resonance, hybrid capture, antibodies, direct labeling of the nucleic acid (e.g., end-labeling, labeled tagmentation payloads), non-specific labeling with intercalating dyes (e.g., ethidium bromide, SYBR dyes), or any other known methodology for nucleic acid detection. In some embodiments, a target protein or peptide fragment may be detected using absorbance, fluorescence, mass spectroscopy, amino acid sequencing, or any other known methodology for protein or peptide detection.

### Sample Preparation Devices and Modules

Devices or modules including apparatuses, cartridges (e.g., comprising channels (e.g., microfluidic channels)), and/or pumps (e.g., peristaltic pumps) for use in a process of preparing a sample for analysis are generally provided. Devices can be used in accordance with the instant disclosure to promote capture, concentration, manipulation, and/or detection of a target molecule from a biological sample. In some embodiments, devices and related methods are provided for automated processing of a sample to produce material for next generation sequencing and/or other downstream analytical techniques. Devices and related methods may be used for performing chemical and/or biological reactions, including reactions for nucleic acid and/or protein processing in accordance with sample preparation or sample analysis processes described elsewhere herein.

A sample preparation device or module may, in some embodiments, perform any number of the following sample preparation steps:

(1) Cell or tissue preparation (e.g., lysis); and/or

(2) Enrichment of at least one target molecule (e.g., at least one target nucleic acid and/or at least one target protein); and/or

(3) Digestion or fragmentation of the at least one target molecule (e.g., at least one target nucleic acid and/or at least one target protein); and/or

(4) Terminal functionalization of the at least one target molecule (e.g., C-terminal functionalization of a target protein).

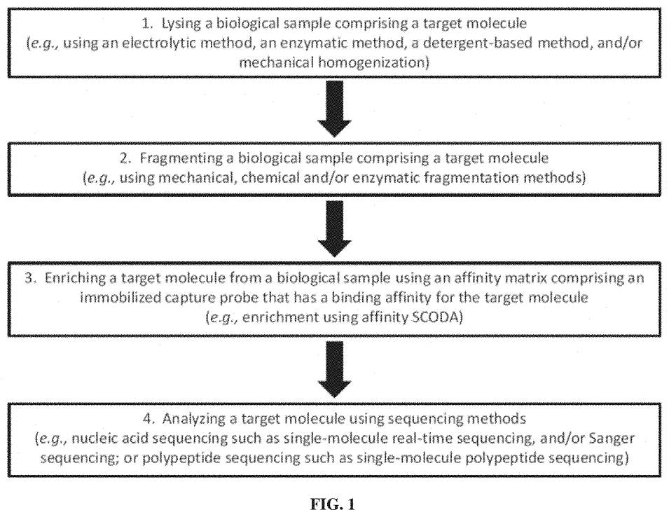

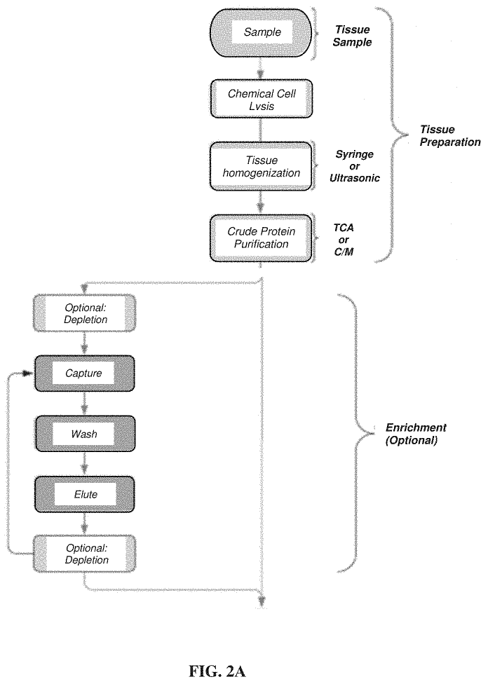

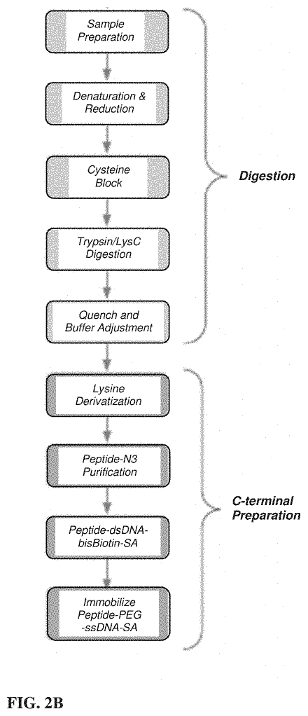

In some embodiments, a sample preparation device or module performs sample preparation steps as shown in FIG. 1. In some embodiments, a sample preparation device or module performs sample preparation steps as shown in FIGS. 2A-2B.

In some embodiments, a sample preparation device or module performs all of steps (1)-(4). In some embodiments, a sample preparation device or module performs step (1) and optionally performs steps (2)-(4). In some embodiments, a sample preparation device or module performs step (1) and optionally performs steps (2)-(3). In some embodiments, a sample preparation device or module performs step (1) and optionally performs step (2). In some embodiments, a sample preparation device or module performs step (1) and optionally performs steps (3)-(4). In some embodiments, a sample preparation device or module performs step (1) and optionally performs step (3). In some embodiments, a sample preparation device or module performs step (1) and optionally performs step (4). In some embodiments, a sample preparation device or module does not perform step (1) and only performs steps (2)-(4). In some embodiments, a sample preparation device or module does not perform step (1) and only performs steps (3)-(4). In some embodiments, a sample preparation device or module does not perform step (1) and only performs steps (2) and (4). In some embodiments, a sample preparation device or module does not perform step (1) and only performs one of steps (2), (3), or (4). The order of steps can be altered as necessary for an experiment. For example, step (3)—digestion or fragmentation—can precede step (2)—enrichment. In some embodiments, the at least one target molecule can be purified after step (1), and/or step (2), and/or step (3), and/or step 4. In some embodiments, any one of the steps is interspersed with manual steps. This flexibility enables the user to address multiple sample types and sequencing platforms.

In some embodiments, a sample preparation device or module is positioned to deliver or transfer to a sequencing module or device a target molecule or a plurality of target molecules (e.g., target nucleic acids or target proteins). In some embodiments, a sample preparation device or module is connected directly to (e.g., physically attached to) or indirectly to a sequencing device or module.

In some embodiments, a sample preparation device or module is used to prepare a sample for diagnostic purposes. In some embodiments, a sample preparation device that is used to prepare a sample for diagnostic purposes is positioned to deliver or transfer to a diagnostic module or diagnostic device a target molecule or a plurality of molecules (e.g., target nucleic acids or target proteins). In some embodiments, a sample preparation device or module is connected directly to (e.g., physically attached to) or indirectly to a diagnostic device.

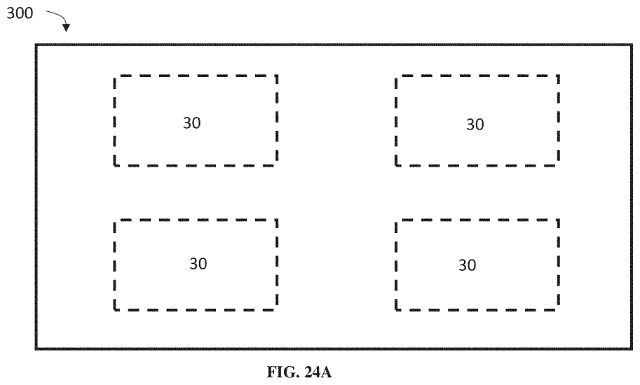

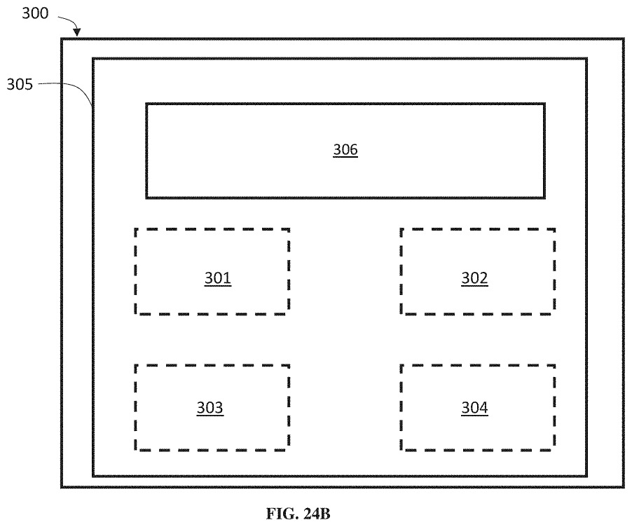

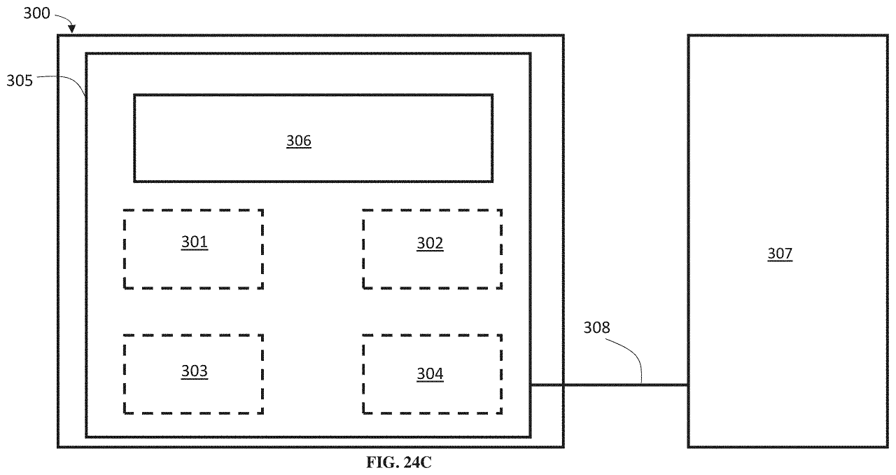

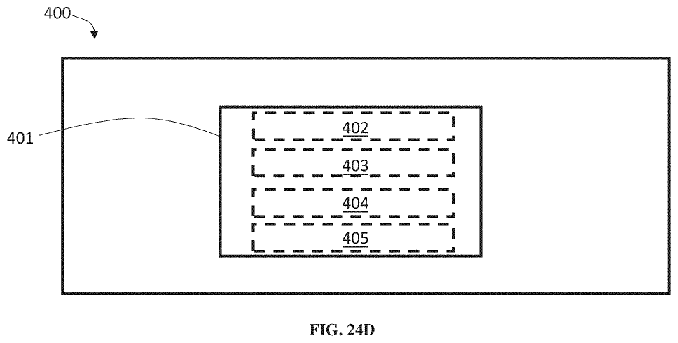

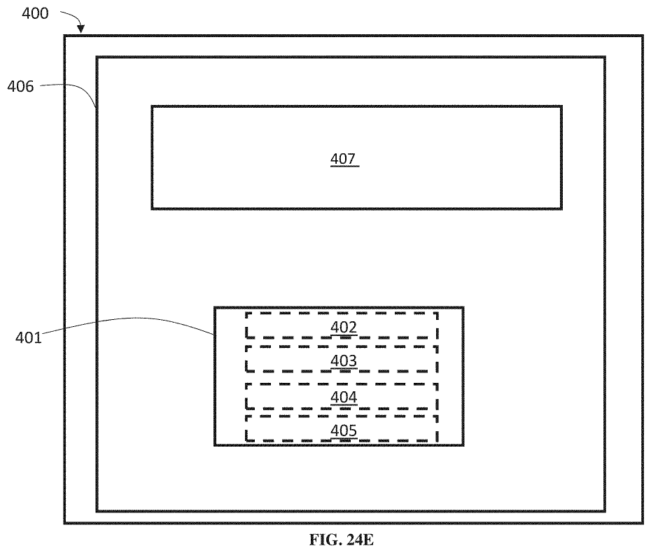

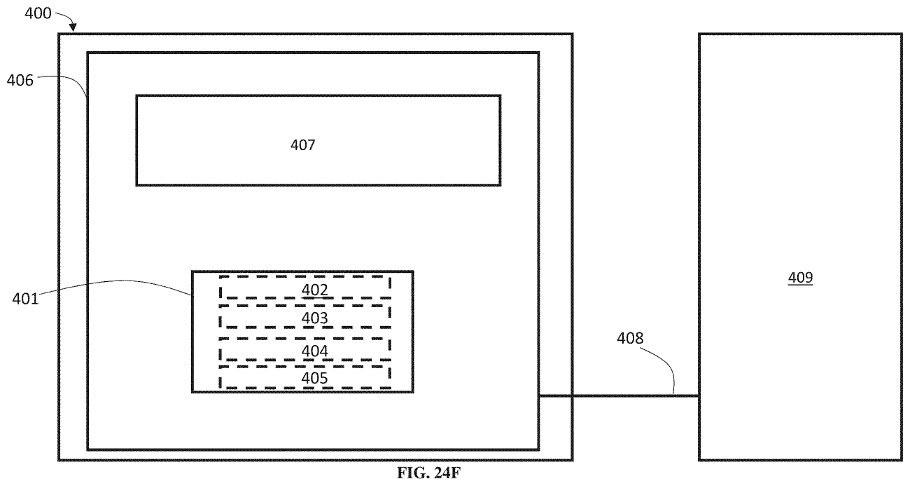

In some embodiments, a device comprises a cartridge housing that is configured to receive one or more cartridges (e.g., configured to receive one cartridge at a time). FIG. 24A shows a schematic diagram of sample preparation device 300, in accordance with some embodiments. A device (e.g., a sample preparation device comprising a cartridge housing) may be configured to receive one or more cartridges (or two or more, or three or more, and so on) either sequentially or simultaneously. Sample preparation device 300, for example, can be configured to receive one or more of lysis cartridge 301, enrichment cartridge 302, fragmentation cartridge 303, and/or functionalization cartridge 304 simultaneously or sequentially. It should be understood that the device need not be configured to receive each of the four cartridges shown in FIG. 4A in all embodiments. For example, in some embodiments sample preparation device 300 is configured to receive only lysis cartridge 301 and enrichment cartridge 302, with fragmentation and functionalization performed manually rather than in an automated fashion.

The sample preparation device may further comprise a pump configured to transport components (e.g., reagents, samples) in the received cartridges (e.g., within a channels/reservoirs of a cartridge or into and/or out of a cartridge). For example, referring to FIG. 24B, sample preparation device 300 may comprise pump 305 configured to transport components in one or more of lysis cartridge 301, enrichment cartridge 302, fragmentation cartridge 303, and/or functionalization cartridge 304. In some embodiments, a pump comprises an apparatus and a received cartridge, and an interaction between the apparatus of the pump and cartridge causes fluid flow. For example, pump 305 may be a peristaltic pump, and apparatus 306 may operatively couple to a cartridge (e.g., cartridge 301) to cause fluid motion in the cartridge (e.g., when apparatus 306 comprises a roller and cartridge 301 comprises a flexible surface deformable by the roller). Further description of exemplary peristaltic pump methods and devices are described in more detail below.

As mentioned elsewhere, a prepared sample from the sample preparation device may be transported (directly or indirectly) to a downstream detection module (e.g., a sequencing module, a diagnostic module). For example, FIG. 24C shows an embodiment in which conduit 308 connects sample preparation device 300 and detection module 307 (e.g., a sequencing module). Sample preparation device 300 and detection module 307 may be directly connected (e.g., physically attached) or may be connected indirectly (e.g., via one or more intervening modules).

While in some embodiments various steps of the processes are performed in separate cartridges (e.g., a lysis step in a lysis cartridge, an enrichment step in an enrichment cartridge, a fragmentation step in a fragmentation cartridge, a functionalization step in a functionalization cartridge), in other embodiments two or more (or all) such steps may be performed in a single cartridge. For example, a cartridge may comprise different regions for different steps of an overall process (each region comprising various reservoirs, channels, and/or microchannels for performing a respective step). FIG. 24D depicts a schematic illustration of one such embodiment, where cartridge 401 comprises lysis region 402, enrichment region 403, fragmentation region 404, and functionalization region 405. It should be understood that while cartridge 401 shows regions for four such steps, the depiction is purely illustrative, and more or fewer regions for more or fewer steps may be present on a given cartridge (e.g., a cartridge may comprise only a lysis region and an enrichment region, or various other combinations). Sample preparation device 400 may be configured to receive cartridge 401, as shown in FIG. 24D according to certain embodiments. As in the embodiments described in FIGS. 24B-24C, sample preparation device 400 may comprise pump 406 comprising apparatus 407 to operatively couple to cartridge 407 (e.g., to transport components such as fluids), as shown in FIG. 24E. Further, as shown in FIG. 24F, conduit 408 can connect sample preparation device 400 to downstream detection module 409 (e.g., a sequencing module, a diagnostic module), in accordance with certain embodiments. Such a connection may allow transportation of a prepared sample from sample preparation device 400 to detection module 409 directly or indirectly, according to certain embodiments.

In some embodiments, a cartridge comprises one or more reservoirs or reaction vessels configured to receive a fluid and/or contain one or more reagents used in a sample preparation process. In some embodiments, a cartridge comprises one or more channels (e.g., microfluidic channels) configured to contain and/or transport a fluid (e.g., a fluid comprising one or more reagents) used in a sample preparation process. Reagents include buffers, enzymatic reagents, polymer matrices, capture reagents, size-specific selection reagents, sequence-specific selection reagents, and/or purification reagents. Additional reagents for use in a sample preparation process are described elsewhere herein.

In some embodiments, a cartridge includes one or more stored reagents (e.g., of a liquid or lyophilized form suitable for reconstitution to a liquid form). The stored reagents of a cartridge include reagents suitable for carrying out a desired process and/or reagents suitable for processing a desired sample type. In some embodiments, a cartridge is a single-use cartridge (e.g., a disposable cartridge) or a multiple-use cartridge (e.g., a reusable cartridge). In some embodiments, a cartridge is configured to receive a user-supplied sample. The user-supplied sample may be added to the cartridge before or after the cartridge is received by the device, e.g., manually by the user or in an automated process. In some embodiments, a cartridge is a sample preparation cartridge. In some embodiments, a sample preparation cartridge is capable of isolating or purifying a target molecule (e.g., a target nucleic acid or target protein) from a sample (e.g., a biological sample).

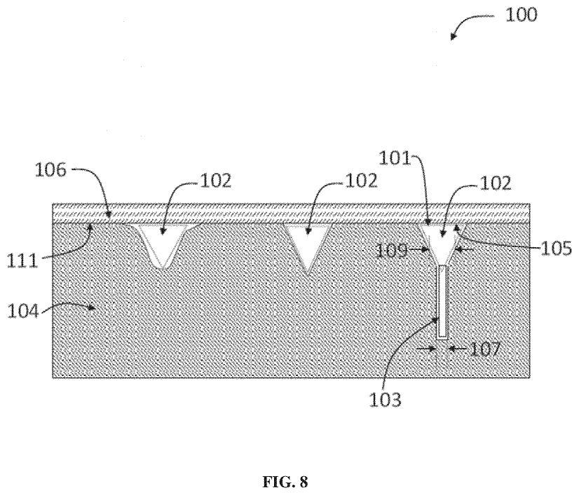

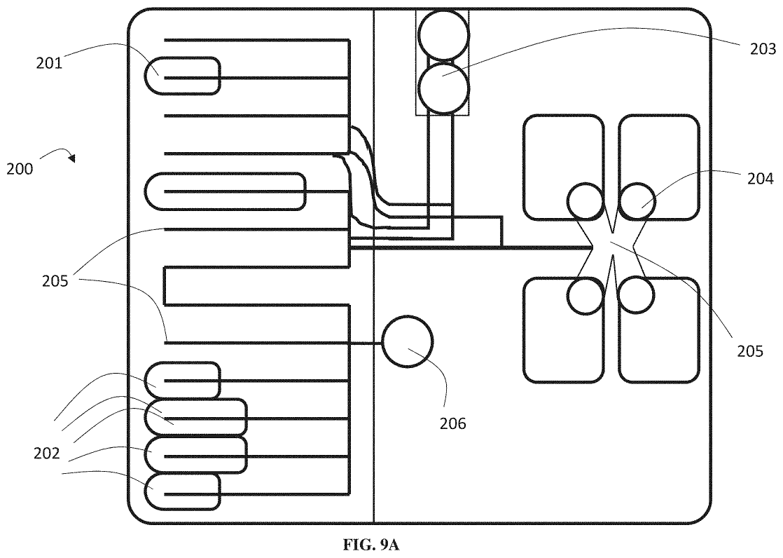

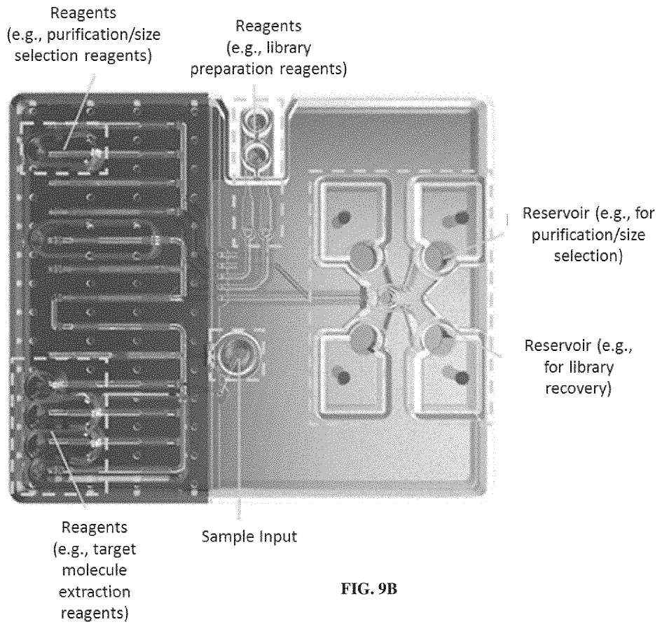

FIG. 9A shows a top view schematic diagram of one embodiment of cartridge 200, in accordance with certain embodiments. Cartridge 200 may be configured to perform one or more of a variety of processes described in this disclosure, such a lysis, enrichment, depletion, fragmentation, and/or terminal functionalization of target molecules from fluid samples (e.g., biological samples). Configuration of a cartridge for any of these processes may be determined, for example, by the presence of reagents selected for the process in the cartridge (e.g., in a reservoir, reaction vessel or channel of the cartridge). For example, cartridge 200 in FIG. 9A can comprise first reagent reservoir 201 comprising or capable of comprising reagents for a first step of a process (e.g., purification/size selection reagents), second reagent reservoirs 202 comprising or capable of comprising reagents for a second step of a process (e.g., target molecule extraction reagents), and third reagent reservoirs 203 comprising or capable of comprising reagents for a third step of a process (e.g., library preparation reagents). Some such reagents may be stored in reservoirs or channels of the cartridge (e.g., a packaged consumable cartridge), or reagents may be introduced into reservoirs or channels of the cartridge prior or during any of the processes described. A sample (e.g., biological sample) may be introduced into the sample via, for example, a sample inlet or port. For example, FIG. 8 shows sample input 206, through which a biological sample may be introduced to a network of channels 205 (e.g., in the form of microchannels) of cartridge 200. Reagents from any of the reservoirs (e.g., first reagent reservoir 201, etc.) may be made to flow through channels 205 to a desired region of cartridge 200 to perform a desire step of a process (e.g., lysis, enrichment, fragmentation, functionalization). For example, reagents for purification/size selection may be made to flow from first reagent reservoir 201 to fourth reservoir 204, and the sample may be made to flow from sample input 206 to fourth reservoir 204, and upon interaction (e.g., via mixing), a purification process of the sample may proceed in fourth reservoir 204 (e.g., via purification/size selection). Samples and reagents may be made to flow (e.g., through channels) in the cartridge via any of a variety of techniques. One such technique is causing flow via peristaltic pumping. Further description of exemplary peristaltic pumping techniques is described below. Other regions of cartridge may be configured for other steps of a process, such as fifth reservoir 205, which may be configured to perform, for example, library recovery, according to some embodiments. FIG. 9B shows an image of an exemplary cartridge that may be configured to perform one or more processes described herein. It should be understood that cartridge configurations other than that shown in FIG. 9B are possible, and FIG. 9B is shown for illustrative purposes.

In some embodiments, a cartridge comprises an affinity matrix for enrichment as described herein. In some embodiments, a cartridge comprises an affinity matrix for enrichment using affinity SCODA, FIGE, or PFGE. In some embodiments, a cartridge comprises an affinity matrix comprising an immobilized affinity agent that has a binding affinity for a target nucleic acid or target protein.

In some embodiments, a sample preparation device of the disclosure produces (e.g., enriches or purifies) target nucleic acids with an average read-length for downstream sequencing applications that is longer than an average read-length produced using control methods (e.g., Sage BluePippin methods, manual methods (e.g., manual bead-based size selection methods)). In some embodiments, a sample preparation device produces target nucleic acids with an average read-length for sequencing that comprises at least 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides in length. In some embodiments, a sample preparation device produces target nucleic acids with an average read-length for sequencing that comprises 700-3000, 1000-3000, 1000-2500, 1000-2400, 1000-2300, 1000-2200, 1000-2100, 1000-2000, 1000-1900, 1000-1800, 1000-1700, 1000-1600, 1000-1500, 1000-1400, 1000-1300, 1000-1200, 1500-3000, 1500-2500, 1500-2000, or 2000-3000 nucleotides in length.

Devices in accordance with the instant disclosure generally contain mechanical and electronic and/or optical components which can be used to operate a cartridge as described herein. In some embodiments, the device components operate to achieve and maintain specific temperatures on a cartridge or on specific regions of the cartridge. In some embodiments, the device components operate to apply specific voltages for specific time durations to electrodes of a cartridge. In some embodiments, the device components operate to move liquids to, from, or between reservoirs and/or reaction vessels of a cartridge. In some embodiments, the device components operate to move liquids through channel(s) of a cartridge, e.g., to, from, or between reservoirs and/or reaction vessels of a cartridge. In some embodiments, the device components move liquids via a peristaltic pumping mechanism (e.g., apparatus) that interacts with an elastomeric, reagent-specific reservoir or reaction vessel of a cartridge. In some embodiments, the device components move liquids via a peristaltic pumping mechanism (e.g., apparatus) that is configured to interact with an elastomeric component (e.g., surface layer comprising an elastomer) associated with a channel of a cartridge to pump fluid through the channel. Device components can include computer resources, for example, to drive a user interface where sample information can be entered, specific processes can be selected, and run results can be reported.

In some embodiments, a cartridge is capable of handling small-volume fluids (e.g., 1-10 μL, 2-10 μL, 4-10 μL, 5-10 μL, 1-8 μL, or 1-6 μL fluid). In some embodiments, the sequencing cartridge is physically embedded or associated with a sample preparation device or module (e.g., to allow for a prepared sample to be delivered to a reaction mixture for sequencing. In some embodiments, a sequencing cartridge that is physically embedded or associated with a sample preparation device or module comprises microfluidic channels that have fluid interfaces in the form of face sealing gaskets or conical press fits (e.g., Luer fittings). In some embodiments, fluid interfaces can then be broken after delivery of the prepared sample in order to physically separate the sequencing cartridge from the sample preparation device or module.

The following non-limiting example is meant to illustrate aspects of the devices, methods, and compositions described herein. The use of a sample preparation device or module in accordance with the instant disclosure may proceed with one or more of the following described steps. A user may open the lid of the device and insert a cartridge that supports the desired process. The user may then add a sample, which may be combined with a specific lysis solution, to a sample port on the cartridge. The user may then close the device lid, enter any sample specific information via a touch screen interface on the device, select any process specific parameters (e.g., range of desired size selection, desired degree of homology for target molecule capture, etc.), and initiate the sample preparation process run. Following the run, the user may receive relevant run data (e.g., confirmation of successful completion of the run, run specific metrics, etc.), as well as process specific information (e.g., amount of sample generated, presence or absence of specific target sequence, etc.). Data generated by the run may be subjected to subsequent bioinformatics analysis, which can be either local or cloud based. Depending on the process, a finished sample may be extracted from the cartridge for subsequent use (e.g., genomic sequencing, qPCR quantification, cloning, etc.). The device may then be opened, and the cartridge may then be removed.

In some embodiments, the sample preparation module comprises a pump. In some embodiments, the pump is peristaltic pump. Some such pumps comprise one or more of the inventive components for fluid handling described herein. For example, the pump may comprise an apparatus and/or a cartridge. In some embodiments, the apparatus of the pump comprises a roller, a crank, and a rocker. In some such embodiments, the crank and the rocker are configured as a crank-and-rocker mechanism that is connected to the roller. The coupling of a crank-and-rocker mechanism with the roller of an apparatus can, in some cases, allow for certain of the advantages describe herein to be achieved (e.g., facile disengagement of the apparatus from the cartridge, well-metered stroke volumes). In certain embodiments, the cartridge of the pump comprises channels (e.g., microfluidic channels). In some embodiments, at least a portion of the channels of the cartridge have certain cross-sectional shapes and/or surface layers that may contribute to any of a number of advantages described herein.

One non-limiting aspect of some cartridges that may, in some cases, provide certain benefits is the inclusion of channels having certain cross-sectional shapes in the cartridges. For example, in some embodiments, the cartridge comprises v-shaped channels. One potentially convenient but non-limiting way to form such v-shaped channels is by molding or machining v-shaped grooves into the cartridge. The recognized advantages of including a v-shaped channel (also referred to herein as a v-groove or a channel having a substantially triangularly-shaped cross-section) in certain embodiments in which a roller of the apparatus engages with the cartridge to cause fluid flow through the channels. For example, in some instances, a v-shaped channel is dimensionally insensitive to the roller. In other words, in some instances, there is no single dimension to which the roller (e.g., a wedge shaped roller) of the apparatus must adhere in order to suitably engage with the v-shaped channel. In contrast, certain conventional cross sectional shapes of the channels, such as semi-circular, may require that the roller have a certain dimension (e.g., radius) in order to suitably engage with the channel (e.g., to create a fluidic seal to cause a pressure differential in a peristaltic pumping process). In some embodiments, the inclusion of channels that are dimensionally insensitive to rollers can result in simpler and less expensive fabrication of hardware components and increased configurability/flexibility.

In certain aspects, the cartridges comprise a surface layer (e.g., a flat surface layer). One exemplary aspect relates to potentially advantageous embodiments involving layering a membrane (also referred to herein as a surface layer) comprising (e.g., consisting essentially of) an elastomer (e.g., silicone) above the v-groove, to produce, in effect, half of a flexible tube. FIG. 24 depicts an exemplary cartridge 100 according to certain such embodiments and is described in more detail below. Then, in some embodiments, by deforming the surface layer comprising an elastomer into the channel to form a pinch and by then translating the pinch, negative pressure can be generated on the trailing edge of the pinch which creates suction and positive pressure can be generated on the leading edge of the pinch, pumping fluid in the direction of the leading edge of the pinch. In certain embodiments, this pumping by interfacing a cartridge (comprising channels having a surface layer) with an apparatus comprising a roller, which apparatus is configured to carry out a motion of the roller that includes engaging the roller with a portion of the surface layer to pinch the portion of the surface layer with the walls and/or base of the associated channel, translating the roller along the walls and/or base of the associated channel in a rolling motion to translate the pinch of the surface layer against the walls and/or base, and/or disengaging the roller with a second portion of the surface layer. In certain embodiments, a crank-and-rocker mechanism is incorporated into the apparatus to carry out this motion of the roller.

A conventional peristaltic pump generally involves tubing having been inserted into an apparatus comprising rollers on a rotating carriage, such that the tubing is always engaged with the remainder of the apparatus as the pump functions. By contrast, in certain embodiments, channels in cartridges herein are linear or comprise at least one linear portion, such that the roller engages with a horizontal surface. In certain embodiments, the roller is connected to a small roller arm that is spring-loaded so that the roller can track the horizontal surface while continuously pinching a portion of the surface layer. Spring loading the apparatus (e.g., a roller arm of the apparatus) can in some cases help regulate the force applied by the apparatus (e.g., roller) to the surface layer and a channel of a cartridge.

In certain embodiments, each rotation of the crank in a crank-and-rocker mechanism connected to the roller provides a discrete pumping volume. In certain embodiments, it is straightforward to park the apparatus in a disengaged position, where the roller is disengaged from any cartridge. In certain embodiments, forward and backward pumping motions are fairly symmetrical as provided by apparatuses described herein, such that a similar amount of force (torque) (e.g., within 10%) is required for forward and backward pumping motions.

In certain embodiments, it may be advantageous to, for a particular size of apparatus, have a relatively high crank radius (e.g., greater than or equal to 2 mm, optionally including associated linkages). Consequently, it may, in certain embodiments, also be advantageous to have a relatively high stroke length (e.g., greater than or equal to 10 mm) to engage with an associated cartridge. Having relatively high crank radius and stroke length, in certain embodiments, ensures no mechanical interference between the apparatus and the cartridge when moving components of the apparatus relative to the cartridge.

In certain embodiments, having v-shaped grooves advantageously allows for utilization with rollers of a variety of sizes having a wedge-shaped edge. By contrast, for example, having a rectangular channel rather than a v-groove results in the width of the roller associated with the rectangular channel needing to be more controlled and precise in relation to the width of the rectangular channel, and results in the forces being applied to the rectangular channel needing to be more precise. Similarly, the channel(s) having a semicircular cross-section may also require more controlled and precise dimension for the width of the associated roller.

In certain embodiments, an apparatus described herein may comprise a multi-axis system (e.g., robot) configured so as to move at least a portion of the apparatus in a plurality of dimensions (e.g., two dimensions, three dimensions). For example, the multi-axis system may be configured so as to move at least a portion of the apparatus to any pumping lane location among associated cartridge(s). For example, in certain embodiments, a carriage herein may be functionally connected to a multi-axis system. In certain embodiments, a roller may be indirectly functionally connected to a multi-axis system. In certain embodiments, an apparatus portion, comprising a crank-and-rocker mechanism connected to a roller, may be functionally connected to a multi-axis system. In certain embodiments, each pumping lane may be addressed by location and accessed by an apparatus described herein using a multi-axis system.

### Nucleic Acid Sequencing Process

Some aspects of the instant disclosure further involve sequencing nucleic acids (e.g., deoxyribonucleic acids or ribonucleic acid). In some aspects, compositions, devices, systems, and techniques described herein can be used to identify a series of nucleotides incorporated into a nucleic acid (e.g., by detecting a time-course of incorporation of a series of labeled nucleotides). In some embodiments, compositions, devices, systems, and techniques described herein can be used to identify a series of nucleotides that are incorporated into a template-dependent nucleic acid sequencing reaction product synthesized by a polymerizing enzyme (e.g., RNA polymerase).

Accordingly, also provided herein are methods of determining the sequence of a target nucleic acid. In some embodiments, the target nucleic acid is enriched (e.g., enriched using electrophoretic methods, e.g., affinity SCODA) prior to determining the sequence of the target nucleic acid. In some embodiments, provided herein are methods of determining the sequences of a plurality of target nucleic acids (e.g., at least 2, 3, 4, 5, 10, 15, 20, 30, 50, or more) present in a sample (e.g., a purified sample, a cell lysate, a single-cell, a population of cells, or a tissue).

In some embodiments, a sample is prepared as described herein (e.g., lysed, purified, fragmented, and/or enriched for a target nucleic acid) prior to determining the sequence of a target nucleic acid or a plurality of target nucleic acids present in a sample. In some embodiments, a target nucleic acid is an enriched target nucleic acid (e.g., enriched using electrophoretic methods, e.g., affinity SCODA).

In some embodiments, methods of sequencing comprise steps of: (i) exposing a complex in a target volume to one or more labeled nucleotides, the complex comprising a target nucleic acid or a plurality of nucleic acids present in a sample, at least one primer, and a polymerizing enzyme; (ii) directing one or more excitation energies, or a series of pulses of one or more excitation energies, towards a vicinity of the target volume; (iii) detecting a plurality of emitted photons from the one or more labeled nucleotides during sequential incorporation into a nucleic acid comprising one of the at least one primers; and (iv) identifying the sequence of incorporated nucleotides by determining one or more characteristics of the emitted photons.

In another aspect, the instant disclosure provides methods of sequencing target nucleic acids or a plurality of target nucleic acids present in a sample by sequencing a plurality of nucleic acid fragments, wherein the target nucleic acid(s) comprises the fragments. In certain embodiments, the method comprises combining a plurality of fragment sequences to provide a sequence or partial sequence for the parent nucleic acid (e.g., parent target nucleic acid). In some embodiments, the step of combining is performed by computer hardware and software. The methods described herein may allow for a set of related nucleic acids (e.g., two or more nucleic acids present in a sample), such as an entire chromosome or genome to be sequenced. In some embodiments, a primer is a sequencing primer. In some embodiments, a sequencing primer can be annealed to a nucleic acid (e.g., a target nucleic acid) that may or may not be immobilized to a solid support. A solid support can comprise, for example, a sample well (e.g., a nanoaperture, a reaction chamber) on a chip or cartridge used for nucleic acid sequencing. In some embodiments, a sequencing primer may be immobilized to a solid support and hybridization of the nucleic acid (e.g., the target nucleic acid) further immobilizes the nucleic acid molecule to the solid support. In some embodiments, a polymerase (e.g., RNA Polymerase) is immobilized to a solid support and soluble sequencing primer and nucleic acid are contacted to the polymerase. In some embodiments a complex comprising a polymerase, a nucleic acid (e.g., a target nucleic acid) and a primer is formed in solution and the complex is immobilized to a solid support (e.g., via immobilization of the polymerase, primer, and/or target nucleic acid). In some embodiments, none of the components are immobilized to a solid support. For example, in some embodiments, a complex comprising a polymerase, a target nucleic acid, and a sequencing primer is formed in situ and the complex is not immobilized to a solid support. In some embodiments, sequencing by synthesis methods can include the presence of a population of target nucleic acid molecules (e.g., copies of a target nucleic acid) and/or a step of amplification (e.g., polymerase chain reaction (PCR)) of a target nucleic acid to achieve a population of target nucleic acids. However, in some embodiments, sequencing by synthesis is used to determine the sequence of a single nucleic acid molecule in any one reaction that is being evaluated and nucleic acid amplification may not be required to prepare the target nucleic acid. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells (e.g., nanoapertures, reaction chambers) on a single chip or cartridge.

In some embodiments, sequencing of a target nucleic acid molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) nucleotides of the target nucleic acid. In some embodiments, the at least two nucleotides are contiguous nucleotides. In some embodiments, the at least two amino acids are non-contiguous nucleotides. In some embodiments, sequencing of a target nucleic acid comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all nucleotides in the target nucleic acid. For example, in some embodiments, sequencing of a target nucleic acid comprises identification of less than 100% of one type of nucleotide in the target nucleic acid. In some embodiments, sequencing of a target nucleic acid comprises identification of less than 100% of each type of nucleotide in the target nucleic acid.

### Terminal Functionalization

A target molecule may be functionalized at a terminal end or position. For example, a target protein may be functionalized at its N-terminal end or its C-terminal end. A target nucleic acid may be functionalized at its 5′ end or its 3′ end. The nucleobase (e.g., guanidine) or the sugar moiety (e.g., ribose or deoxyribose) may be functionalized.

### C-Terminal Carboxylate Functionalization

In one aspect, the present disclosure provides a method of selective C-terminal functionalization of a peptide, comprising:

a. reacting a plurality of peptides of Formula (I):

P—R(CO₂H)ₙ (I)

or salts thereof;
with a compound of Formula (II):

HX-L₁-R₁ (II)

to obtain a plurality of compounds of Formula (III):

P—R [symbol] CO—X-L₁-R₁]ₙ (III)

or salts thereof; and

b. reacting the plurality of compounds of Formula (III), or salts thereof, with a compound of Formula (IV):

R₂-L₂-Z (IV)

to obtain a plurality of compounds of Formula (V):

P—R [symbol] CO—X-L₁-Y-L₂-Z]ₙ (V)

or salts thereof; wherein m, n, P, R(CO₂H)ₙ, HX, X, L₁, L₂, R₁, R₂, Y and Z are defined as follows.

m is an integer of 1-25, inclusive. In certain embodiments, m is 1-10, inclusive. In certain embodiments, m is 5-10, inclusive. In certain embodiments, m is 1-5, inclusive. In certain embodiments, m is 1, 2, 3, 4, 5, 6, 7 8 9, 10, 11, 12, 13, 14, 15, 16, 17, 18, 19, 20, 21, 22, 23, 24, or 25.

n is 1 or 2. In certain embodiments, n is 1. In certain embodiments, n is 2.

Each P independently is a peptide. In certain embodiments, P has 2-100 amino acid residues. In certain embodiments, P has 2-30 amino acid residues.

Each R(CO₂H)ₙ independently is an amino acid residue having n carboxylate moieties. n is 1 or 2. In certain embodiments, n is 1. When n is 1, R(CO₂H)ₙ is lysine or arginine. In a particular embodiment, R(CO₂H)ₙ is lysine. In another particular embodiment, R(CO₂H)ₙ is arginine. In certain embodiments, n is 2. When n is 2, R(CO₂H)ₙ is glutamic acid or aspartic acid. In a particular embodiment, R(CO₂H)ₙ is glutamic acid. In another particular embodiment, R(CO₂H)ₙ is aspartic acid.

HX is nucleophilic moiety that is capable of being acylated, wherein H is a proton. X is one or more heteroatoms. In certain embodiments, X is O, S, or NH, or NO.

L₁ is a linker. In certain embodiments, L₁ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₁ is polyethylene glycol (PEG). In other embodiments, L₁ is a peptide, or an oligonucleotide. In certain embodiments, L₁ is less than 5 nm. In certain embodiments L₁ is less than 1 nm.

L₂ is a linker, or is absent. In certain embodiments, L₂ is absent. In certain embodiments, L₂ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₂ is polyethylene glycol (PEG). In other embodiments, L₂ is a peptide, or an oligonucleotide. In certain embodiments L₂ is between 5-20 nm, inclusive.

R₁ is a moiety comprising a click chemistry handle. In certain embodiments, R₁ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain embodiments, the strained alkene is trans-cyclooctene. In certain embodiments, R₁ is a moiety comprising an azide. In certain embodiments, the tetrazine comprises the structure:

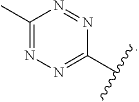 [SMILES: *c1nnc(C)nn1]

R₂ is a moiety comprising a click chemistry handle that is complementary to R₁. The click chemistry handle of R₂ is capable of undergoing a click reaction (i.e., an electrocyclic reaction to form a 5-membered heterocyclic ring) with R₁. For example, when R₁ comprises an azide, nitrile oxide, or a tetrazine, then R₂ may comprise an alkyne or a strained alkene. Conversely, when R₁ comprises an alkyne or a strained alkene, then R₂ may comprise an azide, nitrile oxide, or tetrazine. In certain embodiments, R₂ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain particular embodiments, R₂ comprises BCN. In other particular embodiments, R₂ comprises DBCO. In certain embodiments, the strained alkene is trans-cyclooctene. In certain embodiments, the tetrazine comprises the structure:

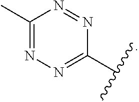 [SMILES: *c1nnc(C)nn1]

Y is a moiety resulting from the click reaction of R₁ and R₂. Y is a 5-membered heterocyclic ring resulting from an electrocyclic reaction (e.g., 3+2 cycloaddition, or 4+2 cycloaddition) between the reactive click chemistry handles of R₁ and R₂. In certain embodiments, Y is a diradical comprising a 1,2,3-triazolyl, 4,5-dihydro-1,2,3-triazolyl, isoxazolyl, 4,5-dihydroisoxazolyl, or 1,4-dihydropyridazyl moiety.

Z is a water-soluble moiety. In certain embodiments, Z imparts water-solubility to the compound to which it is attached. In certain embodiments, Z comprises polyethylene glycol (PEG). In certain embodiments, Z comprises single-stranded DNA. In certain particular embodiments, Z comprises Q24. In certain embodiments, Z comprises double-stranded DNA. In certain embodiments (e.g., compounds of Formula (V)), Z further comprises biotin (e.g., bisbiotin). When Z comprises biotin (e.g., bisbiotin), Z may further comprise streptavidin. In certain embodiments, Z comprises double-stranded DNA. In some embodiments, the moieties of Z are capable of intermolecularly binding another molecule or surface, e.g., to anchor a compound comprising Z to the molecule or surface.

In certain embodiments, the compound of Formula (II) is of Formula (IIa):

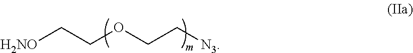

In certain embodiments, Formula (III) is of Formula (IIIa):

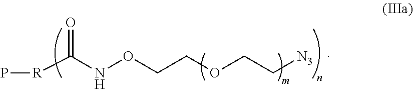

In certain embodiments, n is 1. In certain embodiments, n is 2. In certain embodiments, m is 1. In certain embodiments, m is 5.

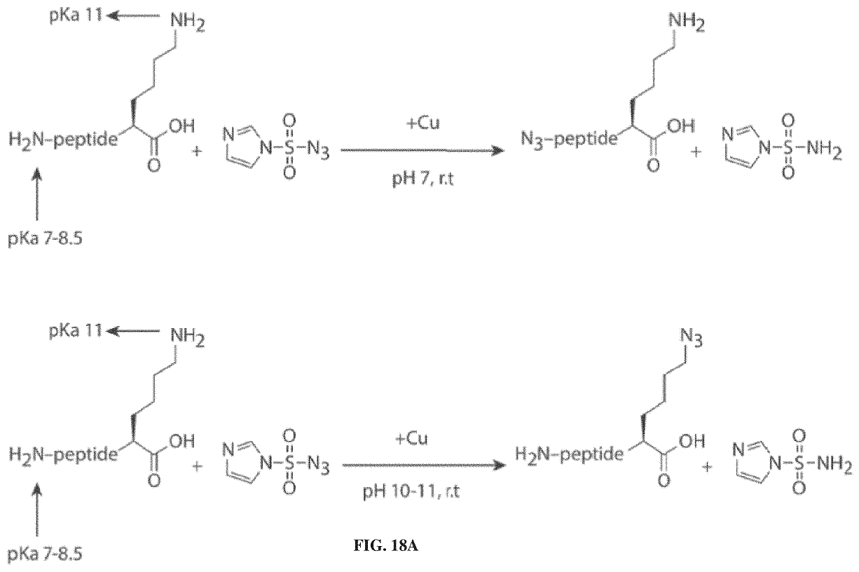

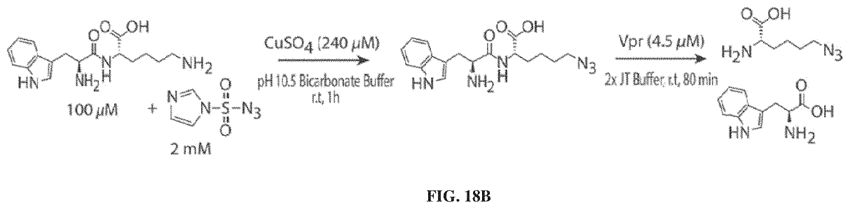

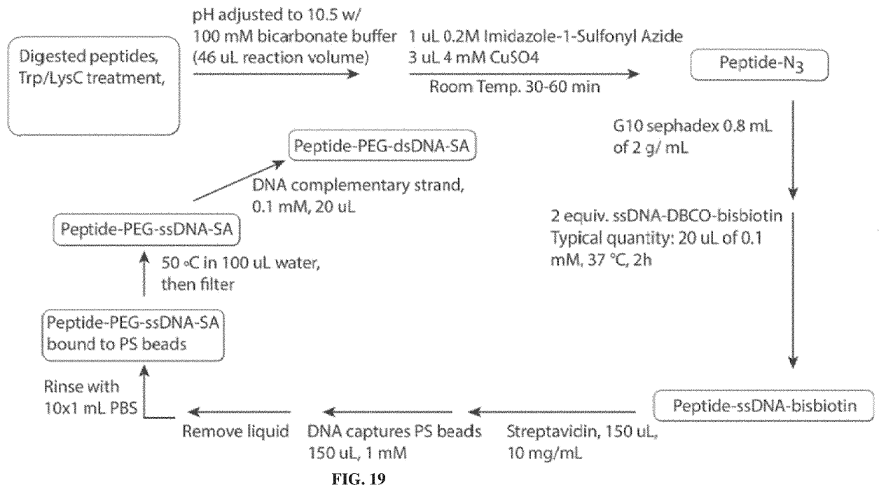

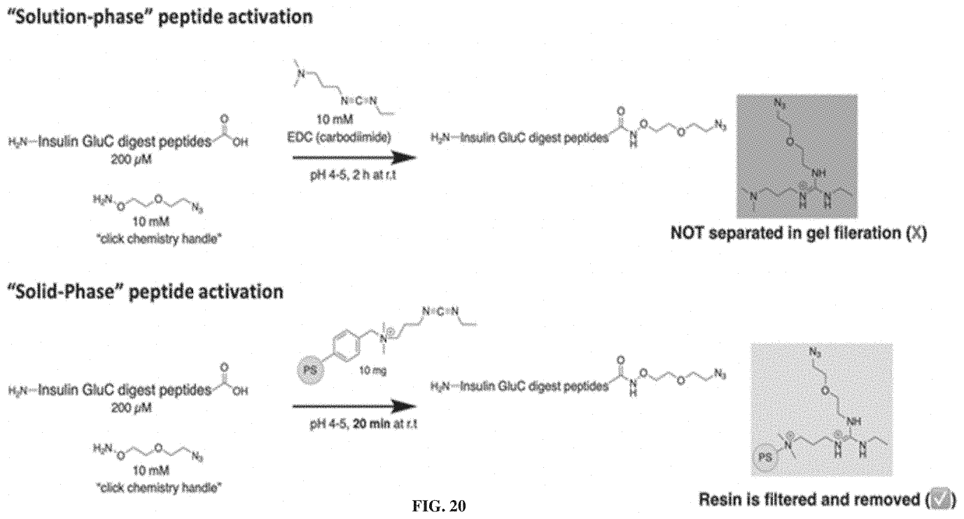

In certain embodiments, Formula (IV) comprises TCO, and single-stranded DNA. In certain embodiments, Formula (IV) further comprises biotin (e.g., bisbiotin). In certain embodiments, Formula (IV) is Q24-BisBt-BCN. In certain embodiments, Formula (IV) is Q24-BisBt-DBCO. In certain embodiments, Formula (IV) is Q24-BisBt-TCO. Generally, Formula (IV) may comprise a branching moiety (e.g., a 1, 3, 5-tricarboxylate moiety), wherein two branches are direct or indirect attachments to biotin moieties, and the third branch is an attachment to the water soluble moiety (e.g., a polynucleotide such as Q24). As shown in FIG. 18B and FIG. 20, in certain embodiments Formula (IV) comprises a triazole moiety derived from the click-coupling of fragments comprising (i) a bisbiotin-azide functionalized linker and (ii) an alkyne (e.g., BCN)-functionalized polynucleotide (e.g. Q24). The click-coupled product may be derivatived to introduce a further click handle R₂, such as BCN or DBCO.

In certain embodiments, Formula (V) is of Formula (Va):

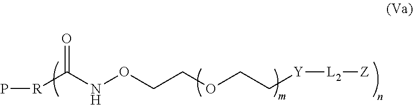

wherein m, n is 1 or 2; and L₂, Y, and Z are as defined above. In certain particular embodiments, n is 1. In certain particular embodiments, n is 2. In certain particular embodiments, m is 1. In certain particular embodiments, m is 5. In certain particular embodiments, L₂ is absent. In certain embodiments, Y comprises a moiety selected from 1,2,3-triazolyl, 4,5-dihydro-1,2,3-triazolyl, isoxazolyl, 4,5-dihydroisoxazolyl, and 1,4-dihydropyridazyl. In certain embodiments, Z comprises single-stranded DNA. In certain embodiments, Z comprises double-stranded DNA. In certain embodiments, Z comprises biotin (e.g., bisbiotin). In certain embodiments, Z further comprises streptavidin.

In certain embodiments, the reaction of step (a) is performed in the presence of a carbodiimide reagent. In certain embodiments, the carbodiimide reagent is water soluble. In a particular embodiment, the carbodiimide reagent is 1-Ethyl-3-(3-dimethylaminopropyl)carbodiimide (EDC). In certain embodiments, the reaction of step (a) is performed at a pH in the range of 3-5. In certain embodiments (e.g., when to total peptide concentration below 1 mM), the concentration of EDC is about 10 mM and the concentration of the compound of Formula (II) is about 20 mM. In certain embodiments (e.g., in connection with Trypsin/LysC digestion, as described below) the concentration of the compound of Formula (II) is about may be about 50 mM and the concentration of EDC may be about 25 mM to suppress C-terminal intramolecular cyclization.

In certain embodiments of step (a), the plurality of compounds of Formula (III) is enriched prior to step (b), for example, by passing the compounds through a G10 sephadex column and/or passing the compounds through a C18 resin column. The use of C18 resin-based enrichment is particularly useful when the compound of Formula (II) is greater than about 200 g/mol. When G-10 sephadex is used in the enrichment, the elution buffer may be 0.5×PBS (pH 7.0). When C18 resin is used in the enrichment, the elution buffer may be 0.1% formic acid with 80% acetonitrile in water. The C18 eluent may be dried and the residue re-suspended in 0.5×PBS prior to step (b).

In certain embodiments, the reaction of step (a) is performed in the presence of an immobilized carbodiimide reagent. For example, the carbodiimide reagent may be covalently attached to a moiety that is stationary and/or insoluble in the reaction solvent, thereby facilitating separation of excess reagent and/or reaction by-products and/or unreacted peptides. See, for example, FIG. 20. In certain embodiments, the immobilized carbodiimide reagent comprises a carbodiimide moiety that is covalently attached to a resin, such as polystyrene (PS). In certain embodiments, the PS-immobilized carbodiimide reagent is of the formula:

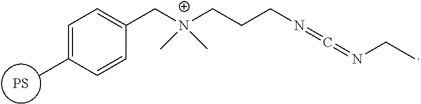

In certain embodiments, when the reaction of step (a) is performed in the presence of an immobilized carbodiimide reagent, for example, a PS-immobilized reagent as described herein, the reaction is performed at a pH in the range of 4 to 5 and/or at ambient temperature and or for about 20 minutes.

In certain embodiments, performing the reaction of step (a) in the presence of an immobilized carbodiimide reagent, for example, a PS-immobilized reagent as described herein, facilitates removal of all unreacted (i.e., non-acylated) peptides because the unreacted peptides remain covalently bound to the immobilized carbodiimide reagent.

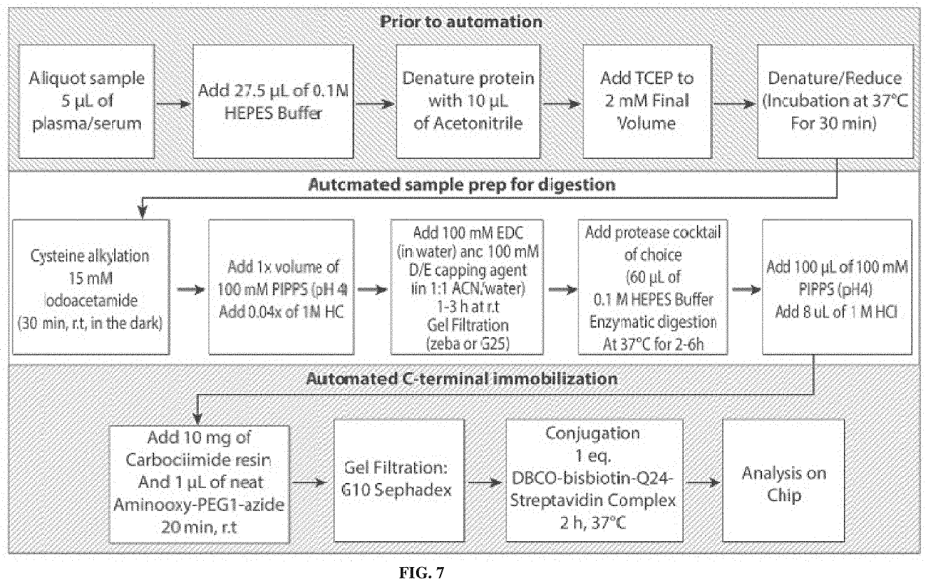

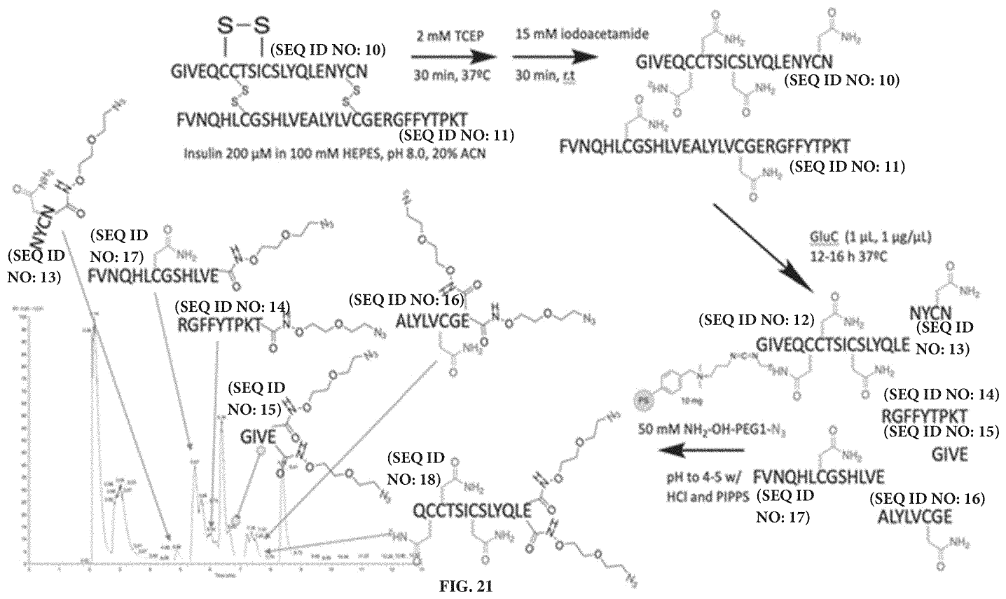

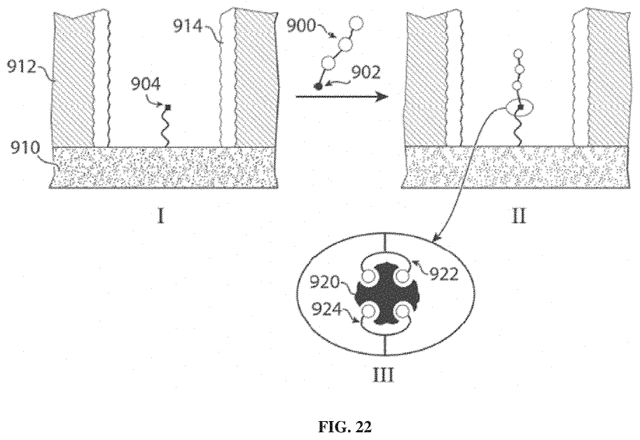

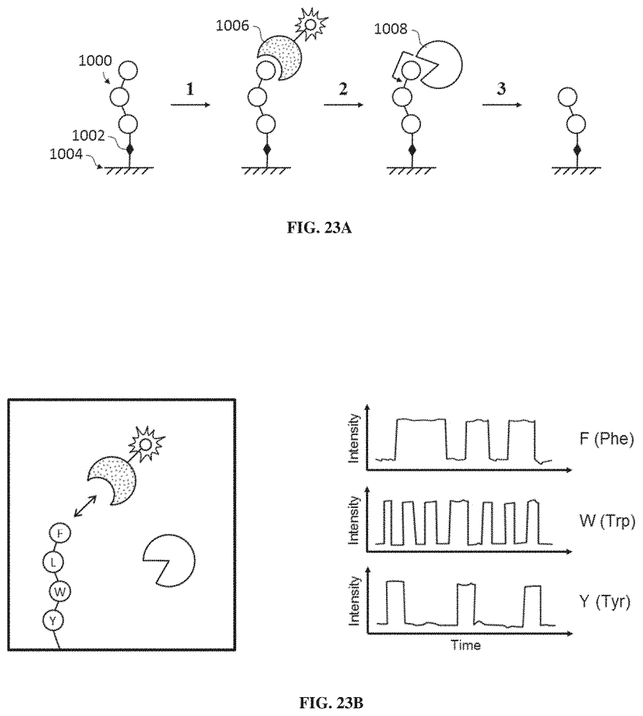

An exemplary process using an immobilized carbodiimide reagent is shown in FIG. 21. An exemplary flowchart for an automation compatible process is shown in FIG. 7.

In certain embodiments of step (b), the click reaction between the plurality of compounds of Formula (III) and the compound of Formula (IV) is uncatalyzed. In certain embodiments, the click reaction is catalyzed, for example, using a copper salt (e.g., a Cu⁺ salt, or a Cu²⁺ salt that is reduced in situ to a Cu⁺ salt). Suitable Cu²⁺ salts include CuSO₄. In certain embodiments, the reaction of step (b) comprises heating the reaction mixture.

In certain embodiments, the compound of Formula (IV) is added to the plurality of compounds of Formula (III). In certain embodiments, the total concentration of the compound of Formula (IV) and the plurality of compounds of Formula (III) is maintained in the range between 10 μM to 1 mM.

In certain embodiments of step (b), when Z comprises single-stranded DNA, the method further comprises hybridizing a complementary DNA strand to the single-stranded DNA to obtain a compound wherein Z comprises double-stranded DNA. In certain embodiments, the single-stranded DNA is Q24 and the complementary DNA strand is Cy3B.

In certain embodiments of step (b), when Z comprises biotin (e.g., bisbiotin), the method further comprises contacting the biotin (e.g., bisbiotin) with streptavidin to obtain a compound wherein Z comprises biotin (e.g., bisbiotin) and streptavidin.

In certain embodiments, the plurality of peptides of Formula (I), or salts thereof, is obtained by subjecting a protein to enzymatic digestion to obtain a digestive mixture comprising the plurality of peptides of Formula (I), or salts thereof. In certain embodiments, the enzymatic digestion comprises cleaving the C-terminal bonds of aspartic acid and/or glutamic acid residues of the protein. In certain specific embodiments, the enzymatic digestion is Glu-C digestion.

In certain embodiments, the total concentration of the plurality of peptides of Formula (I), or salts thereof, after digestion of 20 μg protein is below 100 μM.

In certain embodiments, the enzymatic digestion is performed in phosphate buffer (pH 7.8) or ammonium bicarbonate buffer (pH 4.0).

In certain embodiments, the enzymatic digestion comprises cleaving the C-terminal bonds of lysine and/or arginine residues of the protein. In certain specific embodiments, the enzymatic digestion is Trypsin+Lys-C digestion.

In certain embodiments, the carboxylic acid moieties of the protein, if present, are protected prior to the enzymatic digestion. For example, the carboxylic acid moieties of the protein, if present, may be esterified prior to enzymatic digestion. In certain specific embodiments, the esterified carboxylic acids are methyl esters.

In certain embodiments, the sulfide moieties of the protein are protected prior to enzymatic digestion. In certain specific embodiments, the sulfide moieties are protected by exposing the protein to tris(carboxyethyl)phosphine (TCEP) and iodoacetamide (ICM), or maleimide.

In certain embodiments, the method further comprises the step of enriching the digestive mixture prior to step (a).

### C-Terminal Amine Functionalization

In another aspect, the present disclosure provides a method of selective C-terminal amine functionalization of a peptide, comprising:

a. reacting a plurality of peptides of Formula (VI):

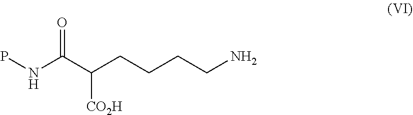

or salts thereof, with a compound of Formula (VII):

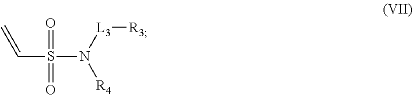

to obtain a plurality of compounds of Formula (VIII):

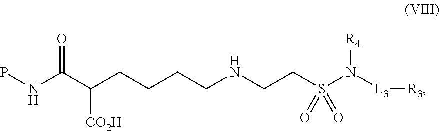

or salts thereof; and

b. reacting the plurality of compounds of Formula (VIII), or salts thereof, with a compound of Formula (IX):

R₅-L₄-Z₁; (IX)

to afford a plurality of compounds of Formula (X):

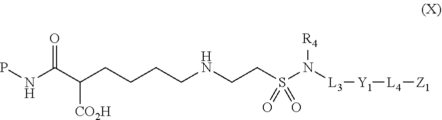

or salts thereof; wherein P, L₃, L₄, R₃, R₄, Y₁, and Z₁ are as defined below.

Each P independently is a peptide. In certain embodiments, P has 2-100 amino acid residues. In certain embodiments, P has 2-30 amino acid residues.

L₃ is a linker. In certain embodiments, L₃ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₃ is polyethylene glycol (PEG). In other embodiments, L₃ is a peptide, or an oligonucleotide.

L₄ is a linker, or is absent. In certain embodiments, L₄ is absent. In certain embodiments, L₄ is a substituted or unsubstituted aliphatic chain, wherein one or more carbon atoms are optionally, independently replaced by a heteroatom, an aryl, heteroaryl, cycloalkyl, or heterocyclyl moiety. In certain embodiments, L₄ is polyethylene glycol (PEG). In other embodiments, L₄ is a peptide, or an oligonucleotide.

R₃ is a moiety comprising a click chemistry handle. In certain embodiments, R₃ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain embodiments, the strained alkene is trans-cyclooctene. In certain embodiments, R₁ is a moiety comprising an azide. In certain embodiments, the tetrazine comprises the structure:

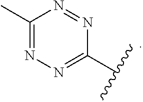 [SMILES: *c1nnc(C)nn1]

R₄ is substituted or unsubstituted aryl or substituted or unsubstituted heteroaryl. In certain embodiments, R₄ is substituted or unsubstituted phenyl. In certain particular embodiments, R₄ is phenyl. In certain particular embodiments, R₄ is 4-nitrophenyl.

R₅ is a moiety comprising a click chemistry handle that is complementary to R₃. The click chemistry handle of R₅ is capable of undergoing a click reaction (i.e., an electrocyclic reaction to form a 5-membered heterocyclic ring) with R₃. For example, when R₃ comprises an azide, nitrile oxide, or a tetrazine, then R₅ may comprise an alkyne or a strained alkene. Conversely, when R₃ comprises an alkyne or a strained alkene, then R₅ may comprise an azide, nitrile oxide, or tetrazine. In certain embodiments, R₅ is a moiety comprising an azide, tetrazine, nitrile oxide, alkyne or strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain particular embodiments, R₅ comprises BCN. In other particular embodiments, R₅ comprises DBCO. In certain embodiments, the strained alkene is trans-cyclooctene. In certain embodiments, the tetrazine comprises the structure:

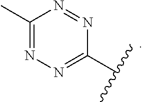 [SMILES: *c1nnc(C)nn1]

Y₁ is a moiety resulting from the click reaction of R₃ and R₅. Y₁ is a 5-membered heterocyclic ring resulting from an electrocyclic reaction (e.g., 3+2 cycloaddition, or 4+2 cycloaddition) between the reactive click chemistry handles of R₃ and R₅. In certain embodiments, Y₁ is a diradical comprising a 1,2,3-triazolyl, 4,5-dihydro-1,2,3-triazolyl, isoxazolyl, 4,5-dihydroisoxazolyl, or 1,4-dihydropyridazyl moiety.

Z₁ is a water-soluble moiety. In certain embodiments, Z₁ imparts water-solubility to the compound to which it is attached. In certain embodiments, Z₁ comprises polyethylene glycol (PEG). In certain embodiments, Z₁ comprises single-stranded DNA. In certain particular embodiments, Z₁ comprises Q24. In certain embodiments, Z₁ comprises single-stranded DNA. In certain embodiments (e.g., compounds of Formula (V)), Z₁ further comprises biotin (e.g., bisbiotin). When Z₁ comprises biotin (e.g., bisbiotin), Z₁ may further comprise streptavidin. In certain embodiments, Z₁ comprises double-stranded DNA. In some embodiments, the moieties of Z₁ are capable of intermolecularly binding another molecule or surface, e.g., to anchor a compound comprising Z₁ to the molecule or surface.

In certain embodiments, the compound of Formula (VII) is selected from:

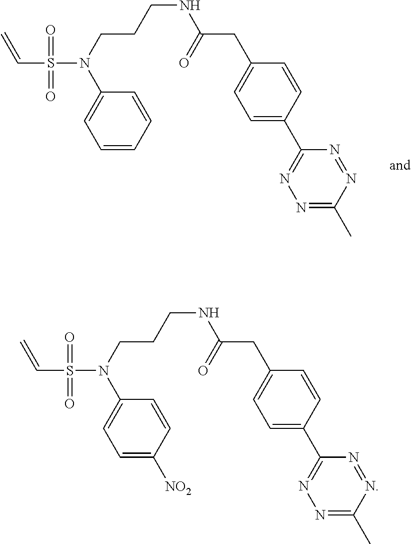

In certain embodiments, Formula (VIII) is of Formula (VIIIa) or Formula (VIIIb):

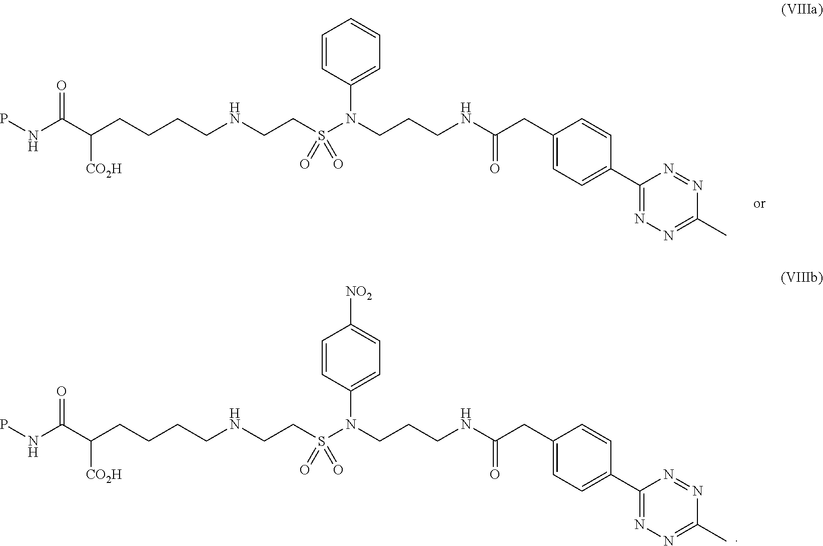

In certain embodiments, Formula (IX) comprises TCO, single-stranded DNA, and biotin (e.g., bisbiotin). In certain embodiments, Formula (IX) is Q24-BisBt-BCN. In certain embodiments, Formula (IX) is Q24-BisBt-DBCO. In certain embodiments, Formula (IX) is Q24-BisBt-TCO. Generally, Formula (IX) may comprise a branching moiety (e.g., a 1, 3, 5-tricarboxylate moiety), wherein two branches are direct or indirect attachments to biotin moieties, and the third branch is an attachment to the water soluble moiety (e.g., a polynucleotide such as Q24). In certain embodiments Formula (IX) comprises a triazole moiety derived from the click-coupling of fragments comprising (i) a bisbiotin-azide functionalized linker and (ii) an alkyne (e.g., BCN)-functionalized polynucleotide (e.g. Q24). The click-coupled product may be derivatived to introduce a further click handle R₅, such as BCN or DBCO.

In certain embodiments, the reaction of step (a) is performed in the presence of a buffer having a concentration in the range of about 20 mM-500 mM and a pH in the range of about 9-11, and acetonitrile in the range of about 20-70% of total volume. In certain embodiments, the reaction of step (a) is performed in pH 9.5 buffer/acetonitrile (1:3 v/v) at approximately 37° C. In certain embodiments, the reaction of step (a) is performed using a concentration of the compound of Formula (VII) of about 500 μM-50 mM.

In certain embodiments, the plurality of compounds of Formula (VIII) is enriched prior to step (b). In certain embodiments, the enrichment comprises ethyl acetate/hexane extraction. Suitable ranges for ethyl acetate/hexane include, but are not limited to, 20 to 100 volume % ethyl acetate in hexanes. In certain embodiments, the volume of organic solvent used in the extraction is about 10× the volume of aqueous layer. Other water immiscible organic solvents can be used in the extraction, e.g., diethyl ether, dichloromethane, chloroform, benzene, toluene, and n−1-butanol.

In certain embodiments, the reaction of step (b) comprises reacting the compounds of Formula (VIII) with about one equivalent of the compound of Formula (IX). In certain embodiments, the reaction of step (b) comprises heating the reaction mixture.

In certain embodiments of step (b), when Z₁ comprises single-stranded DNA, the method further comprises hybridizing a complementary DNA strand to the single-stranded DNA to obtain a compound wherein Z₁ comprises double-stranded DNA. In certain embodiments, the single-stranded DNA is Q24 and the complementary DNA strand is Cy3B.

In certain embodiments of step (b), when Z₁ comprises biotin (e.g., bisbiotin), the method further comprises contacting the biotin (e.g., bisbiotin) with streptavidin to obtain a compound wherein Z₁ comprises biotin (e.g., bisbiotin) and streptavidin.

In certain embodiments, the plurality of peptides of Formula (VI), or salts thereof, is obtained by subjecting a protein to enzymatic digestion to obtain a digestive mixture comprising the plurality of peptides of Formula (VI), or salts thereof. The enzymatic digestion comprises cleaving the C-terminal bonds of lysine and/or arginine residues of the protein. In certain embodiments, the enzymatic digestion is performed using Trypsin, Lys-C, or a combination thereof. In certain embodiments, the enzymatic digestion comprises reacting the protein with Trypsin and Lys-C in Tris-HCl buffer (pH 8.5). In certain embodiments, the total concentration of the plurality of peptides of Formula (VI), or salts thereof, after digestion of 20 μg protein is below 100 μM.

In certain embodiments, the sulfide moieties of the protein are protected prior to enzymatic digestion. In certain specific embodiments, the sulfide moieties are protected by exposing the protein to tris(carboxyethyl)phosphine (TCEP) and iodoacetamide (ICM), or maleimide.

In certain embodiments, the method further comprises the step of enriching the digestive mixture prior to step (a). In certain embodiments, the digestive mixture is used in the method of selective C-terminal amine functionalization of a peptide without enrichment or purification.

### Selective Amine Functionalization Via Diazo Transfer

Prior to sequencing, digested peptides must be functionalized with a moiety that is capable of immobilizing the peptides on the sequencing substrate. Accordingly, the present disclosure provides a method of selective N-functionalization of a peptide, comprising reacting a plurality of peptides of Formula (XI):

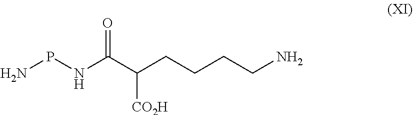

or salts thereof, wherein each P independently is a peptide having an N-terminal amine, with a compound of Formula (XII):

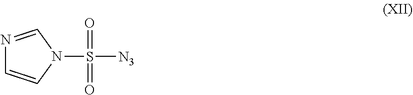 [SMILES: [N-]=[N+]=NS(=O)(=O)n1ccnc1]

under conditions comprising Cu²⁺, or a precursor thereof, and a buffer having a pH of about 10-11; to obtain a plurality of F-azido compounds of the Formula (XIII):

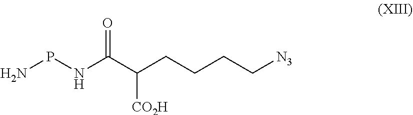

or salts thereof.

Each P independently is a peptide having an N-terminal amine. In certain embodiments, P has 2-100 amino acid residues. In certain embodiments, P has 2-30 amino acid residues. In some embodiments, the concentration of a peptide in the reaction is any conceivable concentration necessary.

In certain embodiments, the Cu²⁺ salt is CuCl₂, CuBr₂, Cu(OH)₂, or CuSO₄. In a particular embodiment, the Cu²⁺ salt is CuSO₄. In certain embodiments, the molar amount of the Cu²⁺ salt is about 2.5 times the molar amount of the compound of Formula (XI). In certain particular embodiments, the concentration of the Cu²⁺ salt is about 250 μM. In some embodiments, the concentration of the Cu²⁺ salt is between 1-5 mM or 100-1000 μM.

In certain embodiments, the conditions further comprise reaction at about 20-30° C., e.g., 20-25° C., 22-27° C., 25-30° C., 20° C., 21° C., 22° C., 23° C., 24° C., 25° C., 26° C., 27° C., 28° C., 29° C., or 30° C.

In certain embodiments, the conditions further comprise reaction for about 30-60 minutes, e.g., 30-35 minutes, 35-40 minutes, 40-45 minutes, 45-50 minutes, 50-55 minutes, or 55-60 minutes.

In certain embodiments, the buffer has a pH of about 10.5. In certain embodiments, the buffer comprises bicarbonate, e.g., sodium bicarbonate. In certain embodiments, the buffer comprises carbonate, e.g., potassium carbonate. In certain embodiments, the buffer comprises phosphate, e.g., potassium phosphate. In some embodiments, the buffer does not comprise an amino group. In some embodiments, the buffer is a Good's buffer (e.g., HEPES, TRIS). In certain embodiments, the buffer has a concentration in the range of 10 mM to 1M, e.g., 10-100 mM, 50-500 mM, 50-100 mM, or 100 mM.

In certain embodiments, the concentration of the compound of Formula (XI) is about 100 μM. In some embodiments, the concentration of the compound of Formula (XI) is about 50 μM. In some embodiments, the concentration of the compound of Formula (XI) is between 1 nM and 1 mM.

In certain embodiments, the amount of the compound of Formula (XII) used in the reaction is 10-30 molar equivalents, e.g., about 20 molar equivalents, relative to the amount of the compound of Formula (XI) used in the reaction. In certain embodiments, the concentration of the compound of Formula (XII) is about 1-3 mM, e.g., about 2 mM.

In certain embodiments, the N-terminal:ε selectivity of the diazo transfer reaction is at least about 90%.

In some embodiments, the method further comprises enriching the plurality of compounds of Formula (XIII), or salts thereof. In certain embodiments, excess compound of Formula (XII) is removed from the reaction mixture using a purification cartridge, e.g., a G-10 sephadex column. In certain embodiments, removal of excess Formula (XIII) using a G-10 sephadex column comprises a buffer exchange to 25 mM HEPES, 25 mM KOAc, pH 7.8.

In some embodiments, the plurality of peptides of Formula (XI), or salts thereof, is obtained by subjecting a protein to enzymatic digestion, as described herein, to obtain a digestive mixture comprising the plurality of peptides of Formula (XI), or salts thereof. The enzymatic digestion comprises cleaving the C-terminal bonds of aspartic acid and/or glutamic acid residues of the protein.

In some embodiments, the enzymatic digestion is Trypsin+Lys-C digestion. In some embodiments, the Trypsin+Lys-C digestion comprises reacting the protein with Trypsin and Lys-C at room temperature in pH 9.5 buffer.

In some embodiments, the method further comprises reacting the plurality of compounds of Formula (XIII) or salts thereof with a DBCO-labeled DNA-streptavidin conjugate, such that the azide moiety of the compounds of Formula (XIII), or salts thereof, undergoes an electrocyclic reaction with the alkyne moiety of DBCO (diarylcyclooctyne) to form a plurality of peptide-DNA-streptavidin conjugates.

In some embodiments, the DBCO-labeled DNA-streptavidin is of Formula (XIV):

R₆-L₅-Z₂ (XIV)

wherein R₆ is DBCO; L₅ is a linker or is absent; and Z₂ is a dsDNA-streptavidin conjugate;

and the plurality of peptide-DNA-streptavidin conjugates are of Formula (XV), or salts thereof:

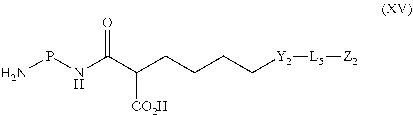

wherein Y₂ is a moiety resulting from a click reaction with the azide moiety of Formula (XIIIb) and R₆.

R₆ is a moiety comprising a click chemistry handle that is complementary to the azide moiety of Formula (XIIIb). The click chemistry handle of R₆ is capable of undergoing a click reaction (i.e., an electrocyclic reaction to form a 5-membered heterocyclic ring) with the azide moiety of Formula (XIIIb). In certain embodiments, R₆ comprises an alkyne or a strained alkene. In certain embodiments, the alkyne is a primary alkyne. In certain embodiments, the alkyne is a cyclic (e.g., mono- or polycyclic) alkyne (e.g., diarylcyclooctyne, or bicycle[6.1.0]nonyne). In certain particular embodiments, R₆ comprises BCN. In other particular embodiments, R₆ comprises DBCO. In certain embodiments, the strained alkene is trans-cyclooctene.

In certain embodiments, L₅ is absent. In certain embodiments, L₅ is a substituted or unsubstituted aliphatic chain, wherein one or more carbon atoms are optionally replaced by a heteroatom, an aryl, heteroaryl, cycloalkyl, or heterocyclyl moiety. In certain embodiments, L₅ is polyethylene glycol (PEG). In other embodiments, L₅ is a peptide, or an oligonucleotide.

In certain embodiments, Z₂ is prepared from a bis-biotin tag which specifically binds to streptavidin in the cis form, leaving the other cis-binding sites free for surface immobilization.

In certain embodiments, Z₂ comprises PEG. In certain embodiments, Z₂ further comprises biotin (e.g., bisbiotin). In certain embodiments, when Z₂ comprises single-stranded DNA, the method further comprises hybridizing a complementary DNA strand to the single-stranded DNA to obtain a compound wherein Z₂ comprises double-stranded DNA. In certain embodiments, the single-stranded DNA is Q24 and the complementary DNA strand is Cy3B.

In certain embodiments, Formula (XIV) is Q24-BisBt-BCN. In certain embodiments, Formula (XIV) is Q24-BisBt-DBCO. In certain embodiments, Formula (XIV) is Q24-BisBt-TCO. Generally, Formula (XIV) may comprise a branching moiety (e.g., a 1, 3, 5-tricarboxylate moiety), wherein two branches are direct or indirect attachments to biotin moieties, and the third branch is an attachment to the water soluble moiety (e.g., a polynucleotide such as Q24). In certain embodiments Formula (XIV) comprises a triazole moiety derived from the click-coupling of fragments comprising (i) a bisbiotin-azide functionalized linker and (ii) an alkyne (e.g., BCN)-functionalized polynucleotide (e.g. Q24). The click-coupled product may be derivatived to introduce a further click handle R₆, such as BCN or DBCO.

In certain embodiments, when Z₂ comprises biotin (e.g., bisbiotin), the method further comprises contacting the biotin (e.g., bisbiotin) with streptavidin to obtain a compound wherein Z₂ comprises biotin (e.g., bisbiotin) and streptavidin.

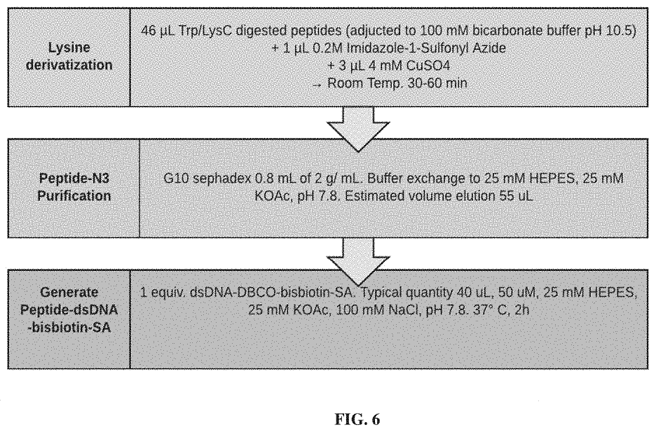

In a particular embodiment, the method of selective N-functionalization of a peptide is carried out according to one or more steps as shown in FIG. 6.

### Click Chemistry

In certain embodiments, the reaction used to conjugate the host to the tag is a “click chemistry” reaction (e.g., the Huisgen alkyne-azide cycloaddition). It is to be understood that any “click chemistry” reaction known in the art can be used to this end. Click chemistry is a chemical approach introduced by Sharpless in 2001 and describes chemistry tailored to generate substances quickly and reliably by joining small units together. See, e.g., Kolb, Finn and Sharpless, Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition).

The term “click chemistry” refers to a chemical synthesis technique introduced by K. Barry Sharpless of The Scripps Research Institute, describing chemistry tailored to generate covalent bonds quickly and reliably by joining small units comprising reactive groups together. See, e.g., Kolb, Finn and Sharpless Angewandte Chemie International Edition (2001) 40: 2004-2021; Evans, Australian Journal of Chemistry (2007) 60: 384-395). Exemplary reactions include, but are not limited to, azide-alkyne Huisgen cycloaddition; and Diels-Alder reactions (e.g., tetrazine [4+2] cycloaddition). In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation).

The term “click chemistry handle,” as used herein, refers to a reactant, or a reactive group, that can partake in a click chemistry reaction. For example, a strained alkyne, e.g., a cyclooctyne, is a click chemistry handle, since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein, for example, in Tables 1 and 2. Other suitable click chemistry handles are known to those of skill in the art.

TABLE 1
Exemplary click chemistry handles and reactions.

| | |
|---|---|
| 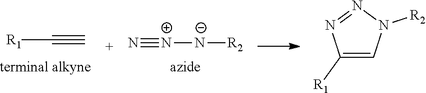 | 1,3-dipolar cycloaddition |
| 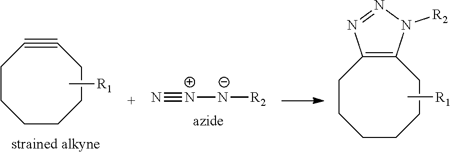 | Strain-promoted cycloaddition |
| 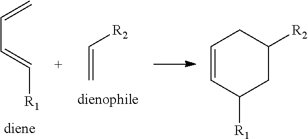 | Diels-Alder reaction |
| 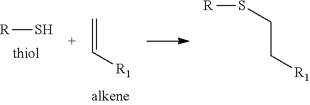 | Thiol-ene reaction |

In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Such click chemistry handles are well known to those of skill in the art and include the click chemistry handles described in Becer, Hoogenboom, and Schubert, Click Chemistry beyond Metal-Catalyzed Cycloaddition, Angewandte Chemie International Edition (2009) 48: 4900-4908.

TABLE 2
Exemplary click chemistry handles and reactions.

| | Reagent A | Reagent B | Mechanism | Notes on reaction^{[a]} |
|---|---|---|---|---|
| 0 | azide | alkyne | Cu-catalyzed [3 + 2] | 2 h at 60° C. in H₂O |
| | | | azide-alkyne cycloaddition | |
| | | | (CuAAC) | |
| 1 | azide | cyclooctyne | strain-promoted [3 + 2] azide-alkyne cycloaddition | 1 h at RT |
| | | | (SPAAC) | |
| 2 | azide | activated | [3 + 2] Huisgen cycloaddition | 4 h at 50° C. |
| | | alkyne | | |
| 3 | azide | electron-deficient | [3 + 2] cycloaddition | 12 h at RT in H₂O |
| | | alkyne | | |
| 4 | azide | aryne | [3 + 2] cycloaddition | 4 h at RT in THF with crown ether or |
| | | | | 24 h at RT in CH₃CN |
| 5 | tetrazine | alkene | Diels-Alder retro-[4 + 2] cycloaddition | 40 min at 25° C. (100% yield) |
| | | | | N₂ is the only by-product |
| 6 | tetrazole | alkene | 1,3-dipolar cycloaddition | few min UV irradiation and then overnight |
| | | | (photoclick) | at 4° C. |
| 7 | dithioester | diene | hetero-Diels-Alder cycloaddition | 10 min at RT |
| 8 | anthracene | maleimide | [4 + 2] Diels-Alder reaction | 2 days at reflux in toluene |
| 9 | thiol | alkene | radical addition | 30 min UV (quantitative conv.) or |
| | | | (thio click) | 24 h UV irradiation (>96%) |
| 10 | thiol | enone | Michael addition | 24 h at RT in CH₃CN |
| 11 | thiol | maleimide | Michael addition | 1 h at 40° C. in THF or |
| | | | | 16 h at RT in dioxane |
| 12 | thiol | para-fluoro | nucleophilic substitution | overnight at RT in DMF or |
| | | | | 60 min 40° C. in DMF |
| 13 | amine | para-fluoro | nucleophilic substitution | 20 min MW at 95° C. in NMP as solvent |

^{[a]}RT = room temperature, DMF = N,N-dimethylformamide, NMP = N-methylpyrolidone, THF = tetrahydrofuran, CH₃CN = acetonitrile.
From Becer, Hoogenboom, and Schubert, Click Chemistry Beyond Metal-Catalyzed Cycloaddition. Angewandte Chemie International Edition (2009) 48: 4900-4908

Additional click chemistry handles suitable for use in methods of conjugation described herein are well known to those of skill in the art, and such click chemistry handles include, but are not limited to, the click chemistry reaction partners, groups, and handles described in PCT/US2012/044584 and references therein, which references are incorporated herein by reference for click chemistry handles and methodology.

### Compounds

In certain aspects, the present disclosure provides compounds of Formulae (II), (IIa), (III), (IIIa), (IV), (V), (Va), (VII), (VIII), (VIIIa), (VIIIb), (XIV), (X), (XI), (XII), (XIIIa), (XIIIb), (XV), and salts thereof, as described herein in various embodiments.

In certain embodiments, the compounds are water soluble.

In certain embodiments, the compounds are useful for applications relating to the analysis of proteins and peptides, such as peptide sequencing. For example, in certain embodiments, compounds of Formulae (V), (X), (XV), and salts thereof, may be covalently or non-covalently attached to a surface.

### Definitions

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense (i.e., as “including, but not limited to”).

Unless defined otherwise, all technical and scientific terms used herein have the same meaning as is commonly understood by one of skill in the art to which this invention belongs.

As used in the specification and claims, the singular form “a”, “an”, and “the” include plural references unless the context clearly dictates otherwise.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”) In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₀ alkyl (such as substituted C₁₋₆ alkyl, e.g., —CH₂F, —CHF₂, —CF₃ or benzyl (Bn)). An alkyl group may be branched or unbranched.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

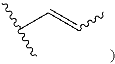

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has Ito 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

“Aralkyl” is a subset of “alkyl” and refers to an alkyl group substituted by an aryl group, wherein the point of attachment is on the alkyl moiety

The term “cycloalkyl” refers to cyclic alkyl radical having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The term “heteroalkyl,” as used herein, refers to an alkyl group, as defined herein, in which one or more of the constituent carbon atoms have been replaced by a heteroatom or optionally substituted heteroatom, e.g., nitrogen

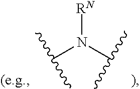

oxygen

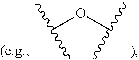 [SMILES: *O*]

or sulfur

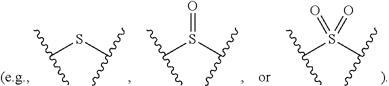

Heteroalkyl groups may be optionally substituted with one, two, three, or, in the case of alkyl groups of two carbons or more, four, five, or six substituents independently selected from any of the substituents described herein. Heteroalkyl group substituents include: (1) carbonyl; (2) halo; (3) C₆-C₁₀ aryl; and (4) C₃-C₁₀ carbocyclyl. A heteroalkylene is a divalent heteroalkyl group.

The term “alkoxy,” as used herein, refers to —OR^{a}, where Ra is, e.g., alkyl, alkenyl, alkynyl, aryl, alkylaryl, carbocyclyl, heterocyclyl, or heteroaryl. Examples of alkoxy groups include methoxy, ethoxy, isopropoxy, tert-butoxy, phenoxy, and benzyloxy.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents (e.g., —F, —OH or —O(C₁₋₆ alkyl). In certain embodiments, the aryl group is an unsubstituted C₆₋₁₄ aryl. In certain embodiments, the aryl group is a substituted C₆₋₁₄ aryl.

The term “aryloxy” refers to an —O-aryl substituent.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “amino,” as used herein, represents —N(R^{N})₂, wherein each R^{N} is, independently, H, OH, NO₂, N(R^{N0})₂, SO₂OR^{N0}, SO₂R^{N0}, SOR^{N0}, an N-protecting group, alkyl, alkoxy, aryl, cycloalkyl, acyl (e.g., acetyl, trifluoroacetyl, or others described herein), wherein each of these recited R^{N} groups can be optionally substituted; or two R^{N} combine to form an alkylene or heteroalkylene, and wherein each R^{N0} is, independently, H, alkyl, or aryl. The amino groups of the disclosure can be an unsubstituted amino (i.e., —NH₂) or a substituted amino (i.e., —N(R^{N})₂).

The term “substituted” as used herein means at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, in some embodiments “substituted” means one or more hydrogen atoms are replaced with NR_{g}Rₕ, NR_{g}C(═O)Rₕ, NR_{g}C(═O)NR_{g}Rₕ, NR_{g}C(═O)ORₕ, NR_{g}SO₂Rₕ, OC(═O)NR_{g}Rₕ, OR_{g}, SR_{g}, SOR_{g}, SO₂R_{g}, OSO₂R_{g}, SO₂OR_{g}, ═NSO₂R_{g}, and SO₂NR_{g}Rₕ. “Substituted also means one or more hydrogen atoms are replaced with C(═O)R_{g}, C(═O)OR_{g}, C(═O)NR_{g}Rₕ, CH₂SO₂R_{g}, CH₂SO₂NR_{g}Rₕ. In the foregoing, R_{g} and Rₕ are the same or different and independently hydrogen, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means one or more hydrogen atoms are replaced by a bond to an aminyl, cyano, hydroxyl, imino, nitro, oxo, thioxo, halo, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

The terms “salt thereof” or “salts thereof” as used herein refer to salts which are well known in the art. For example, Berge et al., describe pharmaceutically acceptable salts in detail in J. Pharmaceutical Sciences, 1977, 66, 1-19, incorporated herein by reference. Additional information on suitable salts can be found in Remington's Pharmaceutical Sciences, 17th ed., Mack Publishing Company, Easton, Pa., 1985, which is incorporated herein by reference.

Salts of the compounds of this invention include those derived from suitable inorganic and organic acids and bases. Examples of acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, lower alkyl sulfonate and aryl sulfonate.

A “protein,” “peptide,” or “polypeptide” comprises a polymer of amino acid residues linked together by peptide bonds. The terms refer to proteins, polypeptides, and peptides of any size, structure, or function. Typically, a protein or peptide will be at least three amino acids in length. In some embodiments, a peptide is between about 3 and about 100 amino acids in length (e.g., between about 5 and about 25, between about 10 and about 80, between about 15 and about 70, or between about 20 and about 40, amino acids in length). In some embodiments, a peptide is between about 6 and about 40 amino acids in length (e.g., between about 6 and about 30, between about 10 and about 30, between about 15 and about 40, or between about 20 and about 30, amino acids in length). In some embodiments, a plurality of peptides can refer to a plurality of peptide molecules, where each peptide molecule of the plurality comprises an amino acid sequence that is different from any other peptide molecule of the plurality. In some embodiments, a plurality of peptides can include at least 1 peptide and up to 1,000 peptides (e.g., at least 1 peptide and up to 10, 50, 100, 250, or 500 peptides). In some embodiments, a plurality of peptides comprises 1-5, 5-10, 1-15, 15-20, 10-100, 50-250, 100-500, 500-1,000, or more, different peptides. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein or peptide may be a fragment of a naturally occurring protein or peptide. A protein may be naturally occurring, recombinant, synthetic, or any combination of these. With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

### Peptide Surface Immobilization

In certain single molecule analytical methods, a molecule to be analyzed is immobilized onto surfaces such that the molecule may be monitored without interference from other reaction components in solution. In some embodiments, surface immobilization of the molecule allows the molecule to be confined to a desired region of a surface for real-time monitoring of a reaction involving the molecule.

Accordingly, in some aspects, the application provides methods of immobilizing a peptide to a surface by attaching any one of the compounds described herein to a surface of a solid support. In some embodiments, the methods comprise contacting a compound of Formula (V), (X), (XV), or a salt thereof, to a surface of a solid support. In some embodiments, the surface is functionalized with a complementary functional moiety configured for attachment (e.g., covalent or non-covalent attachment) to a functionalized terminal end of a peptide. In some embodiments, the solid support comprises a plurality of sample wells formed at the surface of the solid support. In some embodiments, the methods comprise immobilizing a single peptide to a surface of each of a plurality of sample wells. In some embodiments, confining a single peptide per sample well is advantageous for single molecule detection methods, e.g., single molecule peptide sequencing.

As used herein, in some embodiments, a surface refers to a surface of a substrate or solid support. In some embodiments, a solid support refers to a material, layer, or other structure having a surface, such as a receiving surface, that is capable of supporting a deposited material, such as a functionalized peptide described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells.

An example scheme of peptide surface immobilization is depicted in FIG. 9. As shown, panels (I)-(II) depict a process of immobilizing a peptide 900 that comprises a functionalized terminal end 902. In panel (I), a solid support comprising a sample well is shown. In some embodiments, the sample well is formed by a bottom surface comprising a non-metallic layer 910 and side wall surfaces comprising a metallic layer 912. In some embodiments, non-metallic layer 910 comprises a transparent layer (e.g., glass, silica). In some embodiments, metallic layer 912 comprises a metal oxide surface (e.g., titanium dioxide). In some embodiments, metallic layer 912 comprises a passivation coating 914 (e.g., a phosphorus-containing layer, such as an organophosphonate layer). As shown, the bottom surface comprising non-metallic layer 910 comprises a complementary functional moiety 904. Methods of selective surface modification and functionalization are described in further detail in U.S. Patent Publication No. 2018/0326412 and U.S. Provisional Application No. 62/914,356, the contents of each of which are hereby incorporated by reference.

In some embodiments, peptide 900 comprising functionalized terminal end 902 is contacted with complementary functional moiety 904 of the solid support to form a covalent or non-covalent linkage group. In some embodiments, functionalized terminal end 902 and complementary functional moiety 904 comprise partner click chemistry handles, e.g., which form a covalent linkage group between peptide 900 and the solid support. Suitable click chemistry handles are described elsewhere herein. In some embodiments, functionalized terminal end 902 and complementary functional moiety 904 comprise non-covalent binding partners, e.g., which form a non-covalent linkage group between peptide 900 and the solid support. Examples of non-covalent binding partners include complementary oligonucleotide strands (e.g., complementary nucleic acid strands, including DNA, RNA, and variants thereof), protein-protein binding partners (e.g., barnase and barstar), and protein-ligand binding partners (e.g., biotin and streptavidin).

In panel (II), peptide 900 is shown immobilized to the bottom surface through a linkage group formed by contacting functionalized terminal end 902 and complementary functional moiety 904. In this example, peptide 900 is attached through a non-covalent linkage group, which is depicted in the zoomed region of panel (III). As shown, in some embodiments, the non-covalent linkage group comprises an avidin protein 920. Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof. In some embodiments, avidin protein 920 is streptavidin. The multivalency of avidin protein 920 can allow for various linkage configurations, as each of the four binding sites are independently capable of binding a biotin molecule (shown as white circles).

As shown in panel (III), in some embodiments, the non-covalent linkage is formed by avidin protein 920 bound to a first bis-biotin moiety 922 and a second bis-biotin moiety 924. In some embodiments, functionalized terminal end 902 comprises first bis-biotin moiety 922, and complementary functional moiety 904 comprises second bis-biotin moiety 924. In some embodiments, functionalized terminal end 902 comprises avidin protein 920 prior to being contacted with complementary functional moiety 904. In some embodiments, complementary functional moiety 904 comprises avidin protein 920 prior to being contacted with functionalized terminal end 902.

In some embodiments, functionalized terminal end 902 comprises first bis-biotin moiety 922 and a water-soluble moiety, where the water-soluble moiety forms a linkage between first bis-biotin moiety 922 and an amino acid (e.g., a terminal amino acid) of peptide 900. Water-soluble moieties are described in detail elsewhere herein.

### Protein Sequencing Process

Aspects of the instant disclosure also involve methods of protein sequencing and identification, methods of protein sequencing and identification, methods of amino acid identification, and compositions, systems, and devices for performing such methods. Such protein sequencing and identification is performed, in some embodiments, with the same instrument that performs sample preparation and/or genome sequencing, described in more detail herein. In some aspects, methods of determining the sequence of a target protein are described. In some embodiments, the target protein is enriched (e.g., enriched using electrophoretic methods, e.g., affinity SCODA) prior to determining the sequence of the target protein. In some aspects, methods of determining the sequences of a plurality of proteins (e.g., at least 2, 3, 4, 5, 10, 15, 20, 30, 50, or more) present in a sample (e.g., a purified sample, a cell lysate, a single-cell, a population of cells, or a tissue) are described. In some embodiments, a sample is prepared as described herein (e.g., lysed, purified, fragmented, and/or enriched for a target protein) prior to determining the sequence of a target protein or a plurality of proteins present in a sample. In some embodiments, a target protein is an enriched target protein (e.g., enriched using electrophoretic methods, e.g., affinity SCODA)

In some embodiments, the instant disclosure provides methods of sequencing and/or identifying an individual protein in a sample comprising a plurality of proteins by identifying one or more types of amino acids of a protein from the mixture. In some embodiments, one or more amino acids (e.g., terminal amino acids) of the protein are labeled (e.g., directly or indirectly, for example using a binding agent) and the relative positions of the labeled amino acids in the protein are determined. In some embodiments, the relative positions of amino acids in a protein are determined using a series of amino acid labeling and cleavage steps. In some embodiments, the relative position of labeled amino acids in a protein can be determined without removing amino acids from the protein but by translocating a labeled protein through a pore (e.g., a protein channel) and detecting a signal (e.g., a Förster resonance energy transfer (FRET) signal) from the labeled amino acid(s) during translocation through the pore in order to determine the relative position of the labeled amino acids in the protein molecule.

In some embodiments, the identity of a terminal amino acid (e.g., an N-terminal or a C-terminal amino acid) is determined prior to the terminal amino acid being removed and the identity of the next amino acid at the terminal end being assessed; this process may be repeated until a plurality of successive amino acids in the protein are assessed. In some embodiments, assessing the identity of an amino acid comprises determining the type of amino acid that is present. In some embodiments, determining the type of amino acid comprises determining the actual amino acid identity (e.g., determining which of the naturally-occurring 20 amino acids an amino acid is, e.g., using a binding agent that is specific for an individual terminal amino acid). However, in some embodiments, assessing the identity of a terminal amino acid type can comprise determining a subset of potential amino acids that can be present at the terminus of the protein. In some embodiments, this can be accomplished by determining that an amino acid is not one or more specific amino acids (i.e., and therefore could be any of the other amino acids). In some embodiments, this can be accomplished by determining which of a specified subset of amino acids (e.g., based on size, charge, hydrophobicity, binding properties) could be at the terminus of the protein (e.g., using a binding agent that binds to a specified subset of two or more terminal amino acids).

In some embodiments, a protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus).

In some embodiments, a protein is sequenced from its amino (N) terminus. In some embodiments, a protein is sequenced from its carboxy (C) terminus. In some embodiments, a first terminus (e.g., N or C terminus) of a protein is immobilized and the other terminus (e.g., the C or N terminus) is sequenced as described herein.

As used herein, sequencing a protein refers to determining sequence information for a protein. In some embodiments, this can involve determining the identity of each sequential amino acid for a portion (or all) of the protein. In some embodiments, this can involve determining the identity of a fragment (e.g., a fragment of a target protein or a fragment of a sample comprising a plurality of proteins). In some embodiments, this can involve assessing the identity of a subset of amino acids within the protein (e.g., and determining the relative position of one or more amino acid types without determining the identity of each amino acid in the protein). In some embodiments amino acid content information can be obtained from a protein without directly determining the relative position of different types of amino acids in the protein. The amino acid content alone may be used to infer the identity of the protein that is present (e.g., by comparing the amino acid content to a database of protein information and determining which protein(s) have the same amino acid content).

In some embodiments, sequence information for a plurality of protein fragments obtained from a target protein or sample comprising a plurality of proteins (e.g., via enzymatic and/or chemical cleavage) can be analyzed to reconstruct or infer the sequence of the target protein or plurality of proteins present in the sample. Accordingly, in some embodiments, the one or more types of amino acids are identified by detecting luminescence of one or more labeled affinity reagents that selectively bind the one or more types of amino acids. In some embodiments, the one or more types of amino acids are identified by detecting luminescence of a labeled protein.

In some embodiments, the instant disclosure provides compositions, devices, and methods for sequencing a protein by identifying a series of amino acids that are present at a terminus of a protein over time (e.g., by iterative detection and cleavage of amino acids at the terminus). In yet other embodiments, the instant disclosure provides compositions, devices, and methods for sequencing a protein by identifying labeled amino content of the protein and comparing to a reference sequence database.

In some embodiments, the instant disclosure provides compositions, devices, and methods for sequencing a protein by sequencing a plurality of fragments of the protein. In some embodiments, sequencing a protein comprises combining sequence information for a plurality of protein fragments to identify and/or determine a sequence for the protein. In some embodiments, combining sequence information may be performed by computer hardware and software. The methods described herein may allow for a set of related proteins, such as an entire proteome of an organism, to be sequenced. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells on a single chip or cartridge.

In some embodiments, methods provided herein may be used for the sequencing and identification of an individual protein in a sample comprising a plurality of proteins. In some embodiments, the instant disclosure provides methods of uniquely identifying an individual protein in a sample comprising a plurality of proteins. In some embodiments, an individual protein is detected in a mixed sample by determining a partial amino acid sequence of the protein. In some embodiments, the partial amino acid sequence of the protein is within a contiguous stretch of approximately 5-50, 10-50, 25-50, 25-100, or 50-100 amino acids. Without wishing to be bound by any particular theory, it is expected that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a sample comprising a plurality of proteins can be fragmented (e.g., chemically degraded, enzymatically degraded) into short protein fragments of approximately 6 to 40 amino acids, and sequencing of this protein-based library would reveal the identity and abundance of each of the proteins present in the original sample. Compositions and methods for selective amino acid labeling and identifying proteins by determining partial sequence information are described in in detail in U.S. patent application Ser. No. 15/510,962, filed Sep. 15, 2015, entitled “SINGLE MOLECULE PEPTIDE SEQUENCING,” which is incorporated herein by reference in its entirety.

Sequencing in accordance with the instant disclosure, in some aspects, may involve immobilizing a protein (e.g., a target protein) on a surface of a substrate (e.g., of a solid support, for example a chip or cartridge, for example in an sequencing device or module as described herein). In some embodiments, a protein may be immobilized on a surface of a sample well (e.g., on a bottom surface of a sample well) on a substrate. In some embodiments, the N-terminal amino acid of the protein is immobilized (e.g., attached to the surface). In some embodiments, the C-terminal amino acid of the protein is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this disclosure. In some embodiments, a plurality of proteins are attached to a plurality of sample wells (e.g., with one protein attached to a surface, for example a bottom surface, of each sample well), for example in an array of sample wells on a substrate.

In some embodiments, the identity of a terminal amino acid (e.g., an N-terminal or a C-terminal amino acid) is determined, then the terminal amino acid is removed, and the identity of the next amino acid at the terminal end is determined. This process may be repeated until a plurality of successive amino acids in the protein are determined. In some embodiments, determining the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. In some embodiments, determining the identity of a terminal amino acid type can comprise determining a subset of potential amino acids that can be present at the terminus of the protein. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the protein (e.g., using a binding agent that binds to a specified subset of two or more terminal amino acids).

In some embodiments, assessing the identity of a terminal amino acid type comprises determining that an amino acid comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination.

In some embodiments, a protein or protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus).

In some embodiments, sequencing of a protein molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids in the protein molecule. In some embodiments, the at least two amino acids are contiguous amino acids. In some embodiments, the at least two amino acids are non-contiguous amino acids.

In some embodiments, sequencing of a protein molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the protein molecule. For example, in some embodiments, sequencing of a protein molecule comprises identification of less than 100% of one type of amino acid in the protein molecule (e.g., identification of a portion of all amino acids of one type in the protein molecule). In some embodiments, sequencing of a protein molecule comprises identification of less than 100% of each type of amino acid in the protein molecule.

In some embodiments, sequencing of a protein molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the protein.

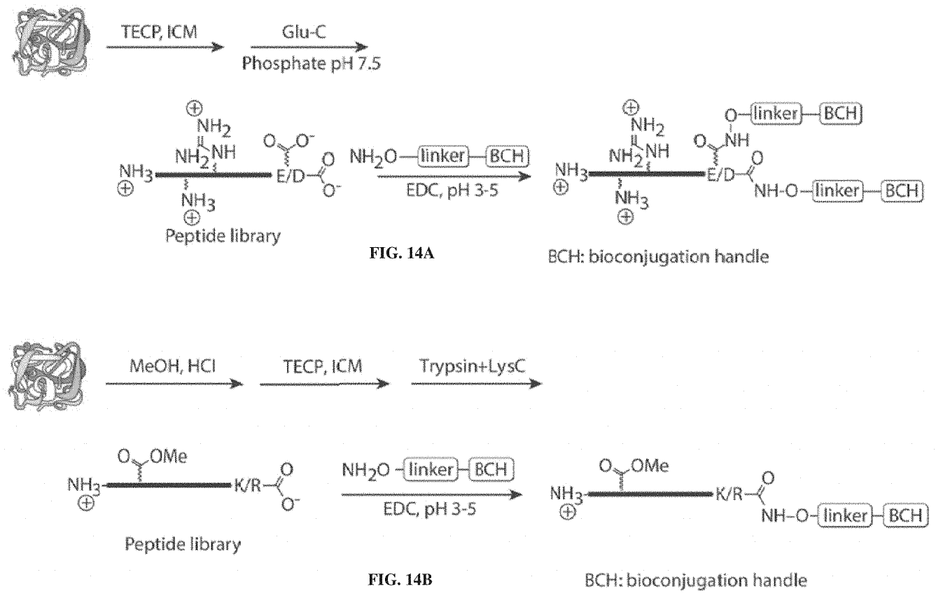

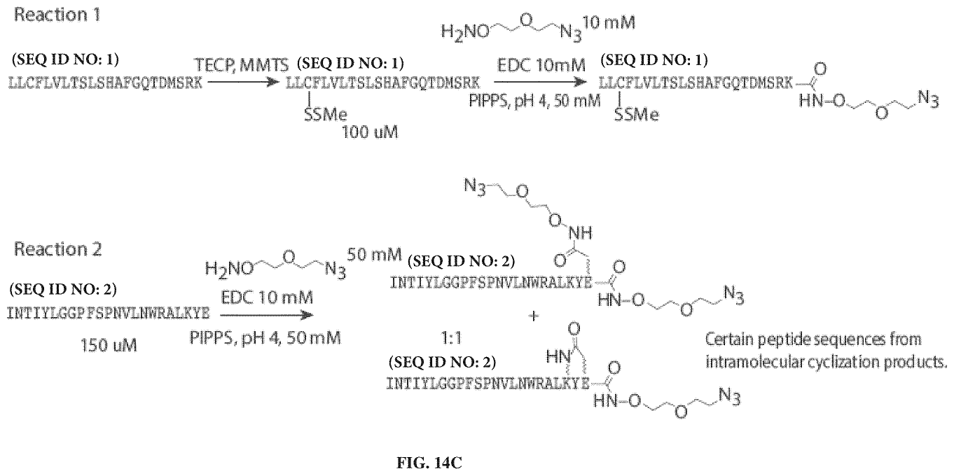

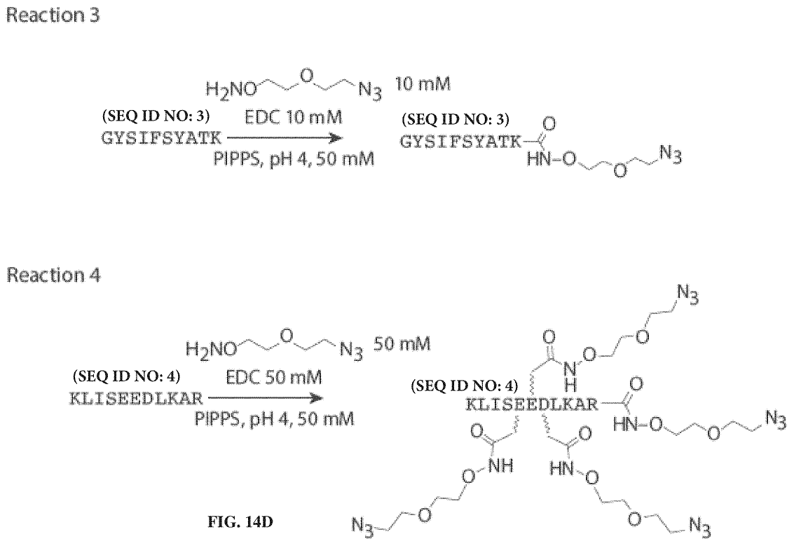

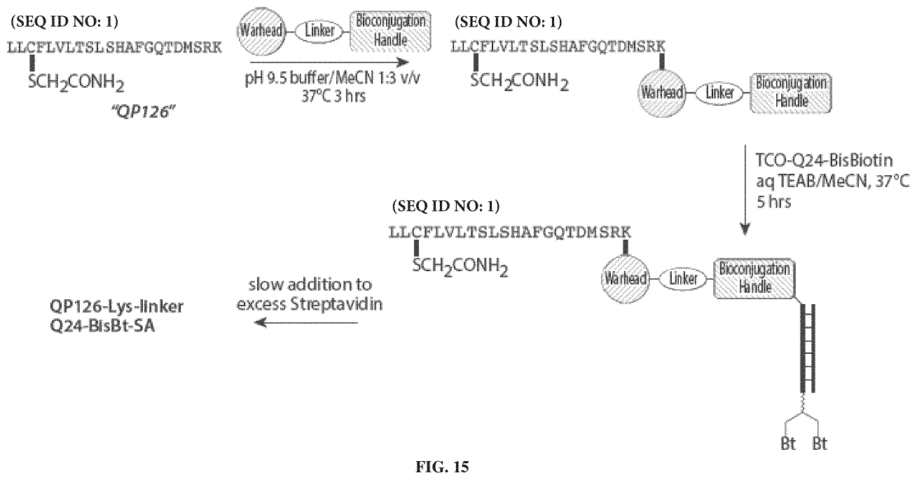

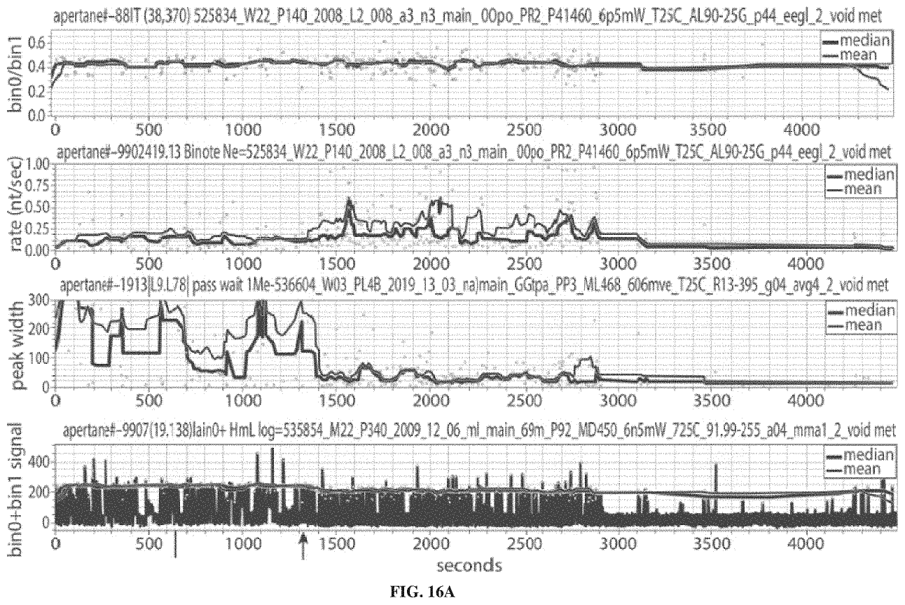

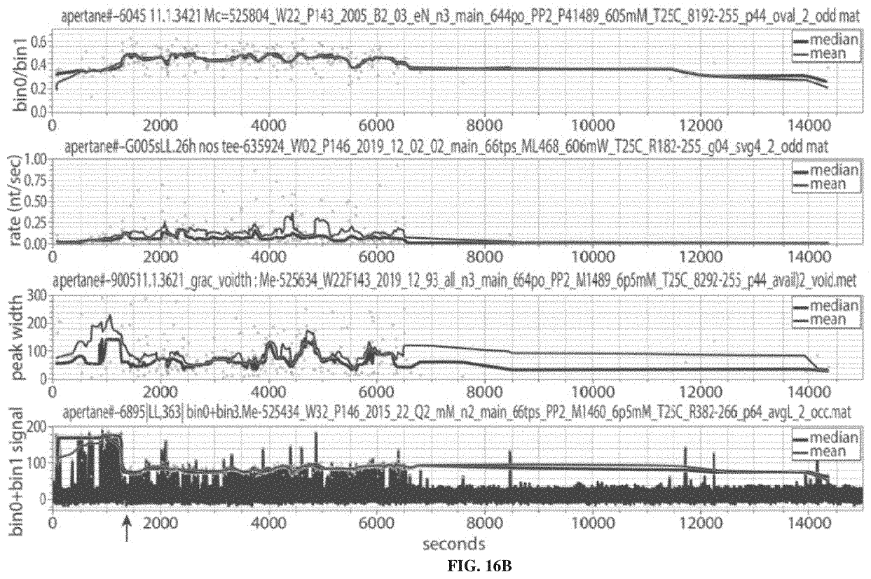

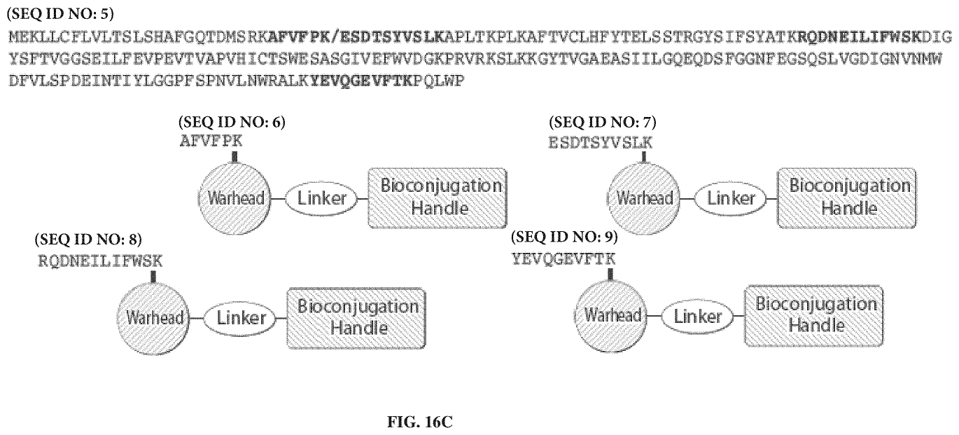

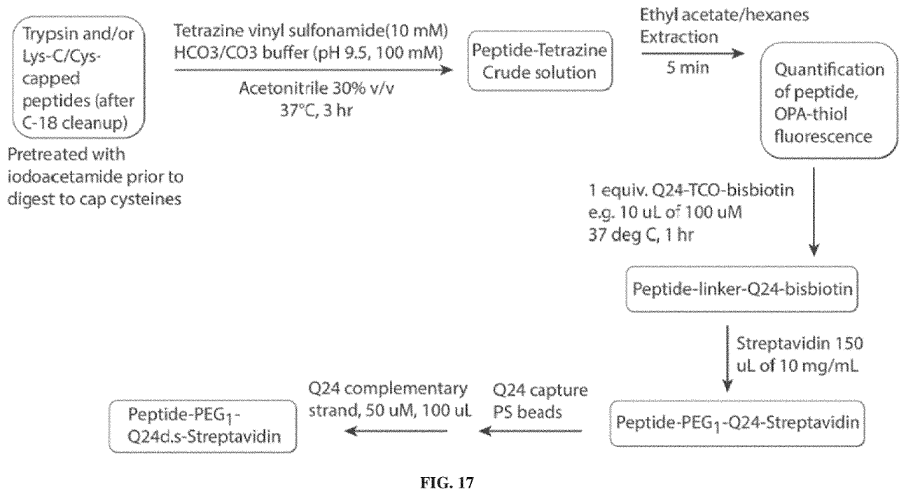

A non-limiting example of protein sequencing by iterative terminal amino acid detection and cleavage is depicted in FIG. 14A. In some embodiments, protein sequencing comprises providing a protein 1000 that is immobilized to a surface 1004 of a solid support (e.g., attached to a bottom or sidewall surface of a sample well) through a linkage group 1002. In some embodiments, linkage group 1002 is formed by a covalent or non-covalent linkage between a functionalized terminal end of protein 1000 and a complementary functional moiety of surface 1004. For example, in some embodiments, linkage group 1002 is formed by a non-covalent linkage between a biotin moiety of protein 1000 (e.g., functionalized in accordance with the disclosure) and an avidin protein of surface 1004. In some embodiments, linkage group 1002 comprises a nucleic acid.

In some embodiments, protein 1000 is immobilized to surface 1004 through a functionalization moiety at one terminal end such that the other terminal end is free for detecting and cleaving of a terminal amino acid in a sequencing reaction. Accordingly, in some embodiments, the reagents used in certain protein sequencing reactions preferentially interact with terminal amino acids at the non-immobilized (e.g., free) terminus of protein 1000. In this way, protein 1000 remains immobilized over repeated cycles of detecting and cleaving. To this end, in some embodiments, linker 1002 may be designed according to a desired set of conditions used for detecting and cleaving, e.g., to limit detachment of protein 1000 from surface 1004. Suitable linker compositions and techniques for functionalizing proteins (e.g., which may be used for immobilizing a protein to a surface) are described in detail elsewhere herein.

In some embodiments, as shown in FIG. 14A, protein sequencing can proceed by (1) contacting protein 1000 with one or more amino acid recognition molecules that associate with one or more types of terminal amino acids. As shown, in some embodiments, a labeled amino acid recognition molecule 1006 interacts with protein 1000 by associating with the terminal amino acid.

In some embodiments, the method further comprises identifying the amino acid (terminal amino acid) of protein 1000 by detecting labeled amino acid recognition molecule 1006. In some embodiments, detecting comprises detecting a luminescence from labeled amino acid recognition molecule 1006. In some embodiments, the luminescence is uniquely associated with labeled amino acid recognition molecule 1006, and the luminescence is thereby associated with the type of amino acid to which labeled amino acid recognition molecule 1006 selectively binds. As such, in some embodiments, the type of amino acid is identified by determining one or more luminescence properties of labeled amino acid recognition molecule 1006.

In some embodiments, protein sequencing proceeds by (2) removing the terminal amino acid by contacting protein 1000 with an exopeptidase 1008 that binds and cleaves the terminal amino acid of protein 1000. Upon removal of the terminal amino acid by exopeptidase 1008, protein sequencing proceeds by (3) subjecting protein 1000 (having n−1 amino acids) to additional cycles of terminal amino acid recognition and cleavage. In some embodiments, steps (1) through (3) occur in the same reaction mixture, e.g., as in a dynamic peptide sequencing reaction. In some embodiments, steps (1) through (3) may be carried out using other methods known in the art, such as peptide sequencing by Edman degradation.

Edman degradation involves repeated cycles of modifying and cleaving the terminal amino acid of a protein, wherein each successively cleaved amino acid is identified to determine an amino acid sequence of the protein. Referring to FIG. 14A, peptide sequencing by conventional Edman degradation can be carried out by (1) contacting protein 1000 with one or more amino acid recognition molecules that selectively bind one or more types of terminal amino acids. In some embodiments, step (1) further comprises removing any of the one or more labeled amino acid recognition molecules that do not selectively bind protein 1000. In some embodiments, step (2) comprises modifying the terminal amino acid (e.g., the free terminal amino acid) of protein 1000 by contacting the terminal amino acid with an isothiocyanate (e.g., PITC) to form an isothiocyanate-modified terminal amino acid. In some embodiments, an isothiocyanate-modified terminal amino acid is more susceptible to removal by a cleaving reagent (e.g., a chemical or enzymatic cleaving reagent) than an unmodified terminal amino acid.

In some embodiments, Edman degradation proceeds by (2) removing the terminal amino acid by contacting protein 1000 with an exopeptidase 1008 that specifically binds and cleaves the isothiocyanate-modified terminal amino acid. In some embodiments, exopeptidase 1008 comprises a modified cysteine protease. In some embodiments, exopeptidase 1008 comprises a modified cysteine protease, such as a cysteine protease from Trypanosoma cruzi (see, e.g., Borgo, et al. (2015) Protein Science 24:571-579). In yet other embodiments, step (2) comprises removing the terminal amino acid by subjecting protein 1000 to chemical (e.g., acidic, basic) conditions sufficient to cleave the isothiocyanate-modified terminal amino acid. In some embodiments, Edman degradation proceeds by (3) washing protein 1000 following terminal amino acid cleavage. In some embodiments, washing comprises removing exopeptidase 1008. In some embodiments, washing comprises restoring protein 1000 to neutral pH conditions (e.g., following chemical cleavage by acidic or basic conditions). In some embodiments, sequencing by Edman degradation comprises repeating steps (1) through (3) for a plurality of cycles.

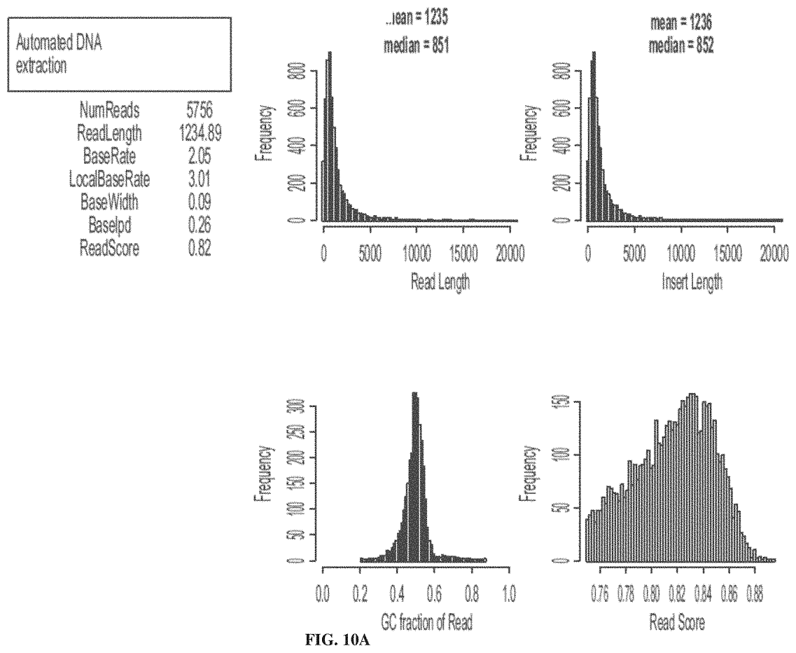

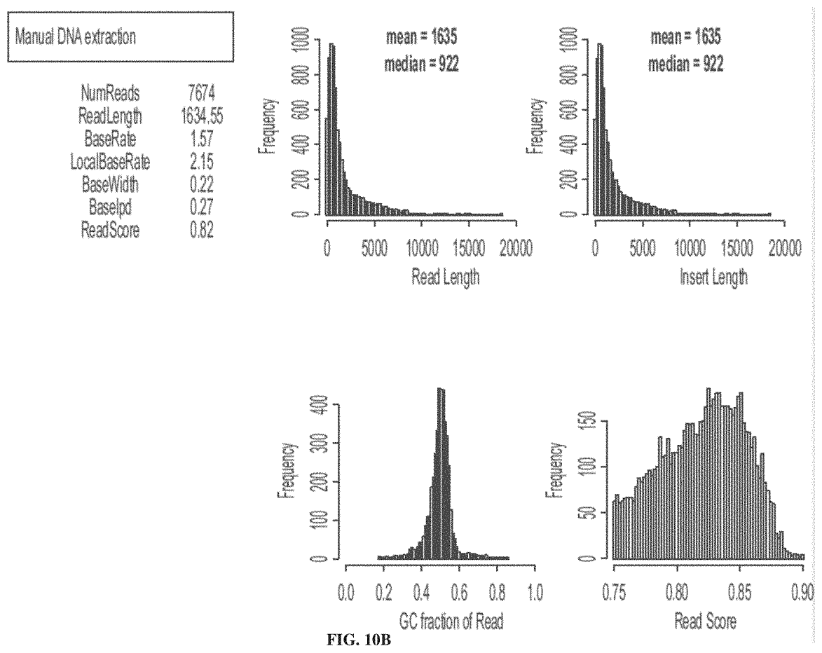

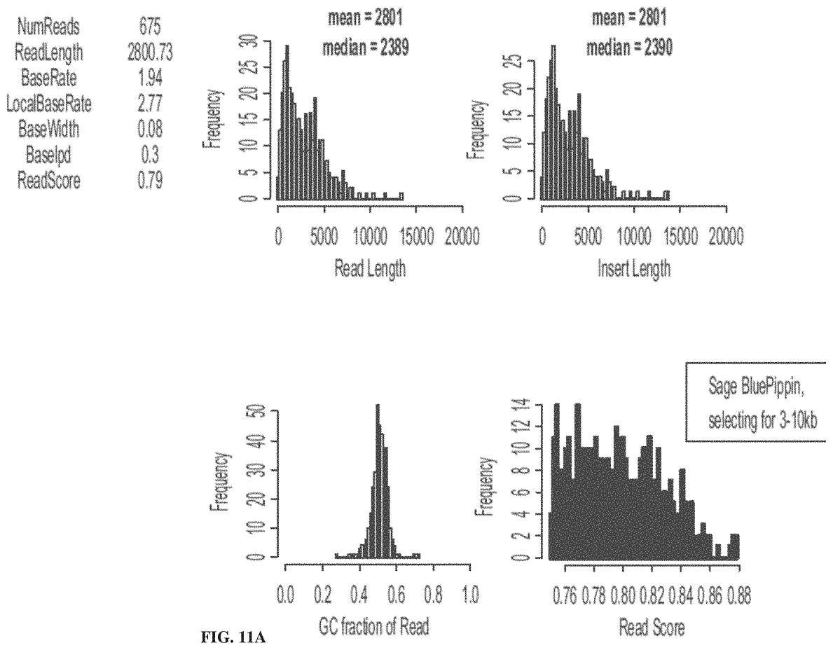

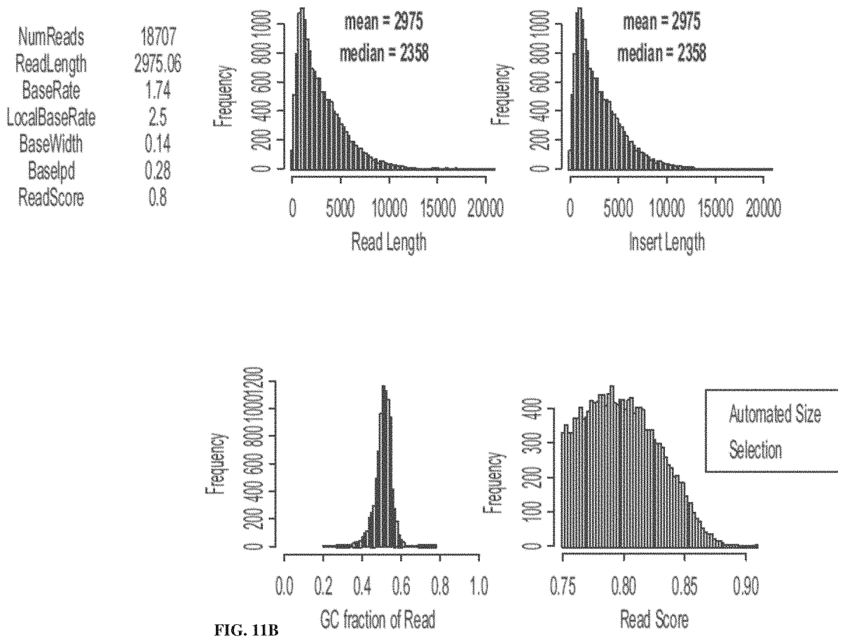

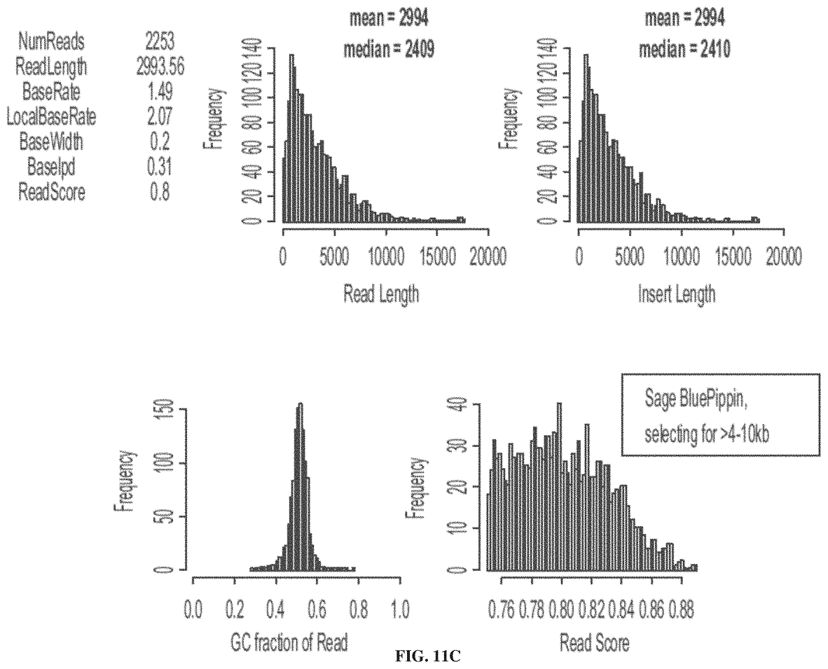

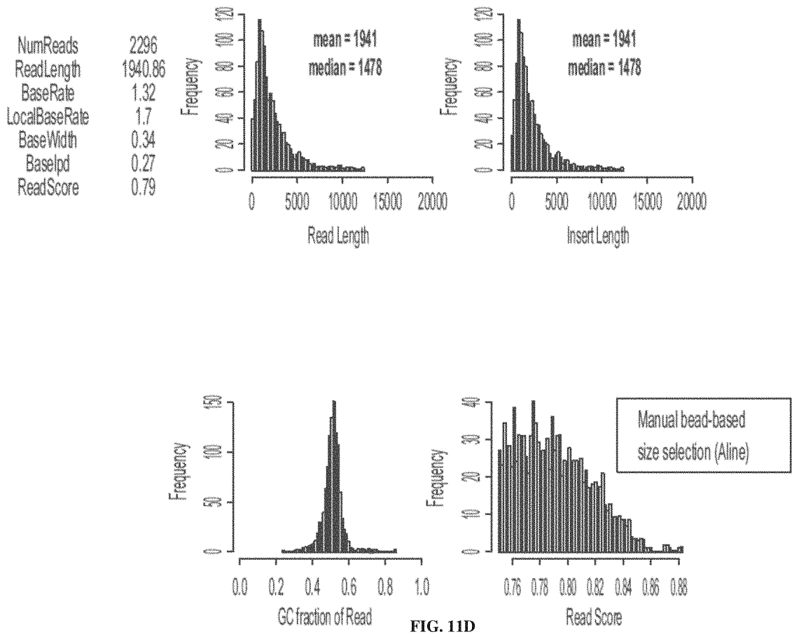

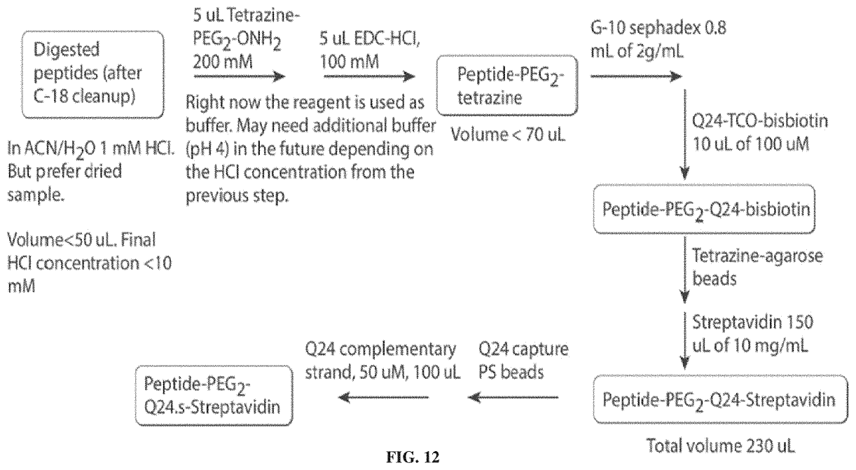

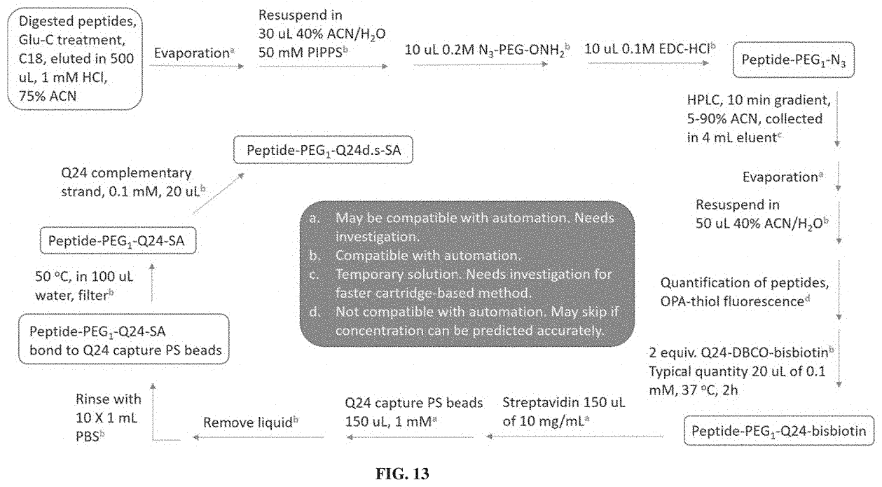

In some embodiments, peptide sequencing can be carried out in a dynamic peptide sequencing reaction. In some embodiments, referring again to FIG. 10A, the reagents required to perform step (1) and step (2) are combined within a single reaction mixture. For example, in some embodiments, steps (1) and (2) can occur without exchanging one reaction mixture for another and without a washing step as in conventional Edman degradation. Thus, in this embodiments, a single reaction mixture comprises labeled amino acid recognition molecule 1006 and exopeptidase 1008. In some embodiments, exopeptidase 1008 is present in the mixture at a concentration that is less than that of labeled amino acid recognition molecule 1006. In some embodiments, exopeptidase 1008 binds protein 1000 with a binding affinity that is less than that of labeled amino acid recognition molecule 1006.

In some embodiments, dynamic protein sequencing is carried out in real-time by evaluating binding interactions of terminal amino acids with labeled amino acid recognition molecules and a cleaving reagent (e.g., an exopeptidase). FIG. 14B shows an example of a method of sequencing in which discrete binding events give rise to signal pulses of a signal output. The inset panel (left) of FIG. 14B illustrates a general scheme of real-time sequencing by this approach. As shown, a labeled amino acid recognition molecule associates with (e.g., binds to) and dissociates from a terminal amino acid (shown here as phenylalanine), which gives rise to a series of pulses in signal output which may be used to identify the terminal amino acid. In some embodiments, the series of pulses provide a pulsing pattern (e.g., a characteristic pattern) which may be diagnostic of the identity of the corresponding terminal amino acid.

As further shown in the inset panel (left) of FIG. 14B, in some embodiments, a sequencing reaction mixture further comprises an exopeptidase. In some embodiments, the exopeptidase is present in the mixture at a concentration that is less than that of the labeled amino acid recognition molecule. In some embodiments, the exopeptidase displays broad specificity such that it cleaves most or all types of terminal amino acids. Accordingly, a dynamic sequencing approach can involve monitoring recognition molecule binding at a terminus of a protein over the course of a degradation reaction catalyzed by exopeptidase cleavage activity.

FIG. 14B further shows the progress of signal output intensity over time (right panels). In some embodiments, terminal amino acid cleavage by exopeptidase(s) occurs with lower frequency than the binding pulses of a labeled amino acid recognition molecule. In this way, amino acids of a protein may be counted and/or identified in a real-time sequencing process. In some embodiments, one type of amino acid recognition molecule can associate with more than one type of amino acid, where different characteristic patterns correspond to the association of one type of labeled amino acid recognition molecule with different types of terminal amino acids. For example, in some embodiments, different characteristic patterns (as illustrated by each of phenylalanine (F, Phe), tryptophan (W, Trp), and tyrosine (Y, Tyr)) correspond to the association of one type of labeled amino acid recognition molecule (e.g., ClpS protein) with different types of terminal amino acids over the course of degradation. In some embodiments, a plurality of labeled amino acid recognition molecules may be used, each capable of associating with different subsets of amino acids.

In some embodiments, dynamic peptide sequencing is performed by observing different association events, e.g., association events between an amino acid recognition molecule and an amino acid at a terminal end of a peptide, wherein each association event produces a change in magnitude of a signal, e.g., a luminescence signal, that persists for a duration of time. In some embodiments, observing different association events, e.g., association events between an amino acid recognition molecule and an amino acid at a terminal end of a peptide, can be performed during a peptide degradation process. In some embodiments, a transition from one characteristic signal pattern to another is indicative of amino acid cleavage (e.g., amino acid cleavage resulting from peptide degradation). In some embodiments, amino acid cleavage refers to the removal of at least one amino acid from a terminus of a protein (e.g., the removal of at least one terminal amino acid from the protein). In some embodiments, amino acid cleavage is determined by inference based on a time duration between characteristic signal patterns. In some embodiments, amino acid cleavage is determined by detecting a change in signal produced by association of a labeled cleaving reagent with an amino acid at the terminus of the protein. As amino acids are sequentially cleaved from the terminus of the protein during degradation, a series of changes in magnitude, or a series of signal pulses, is detected.

In some embodiments, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, different characteristic patterns corresponding to different types of amino acids in a single protein may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). It should be appreciated that, in some embodiments, smaller differences in mean pulse duration between different characteristic patterns may require a greater number of pulse durations within each characteristic pattern to distinguish one from another with statistical confidence.

### Sequencing Device or Module

Sequencing of nucleic acids or proteins in accordance with the instant disclosure, in some aspects, may be performed using a system that permits single molecule analysis. The system may include a sequencing device or module and an instrument configured to interface with the sequencing device or module. The sequencing device or module may include an array of pixels, where individual pixels include a sample well and at least one photodetector. The sample wells of the sequencing device or module may be formed on or through a surface of the sequencing device or module and be configured to receive a sample placed on the surface of the sequencing device or module. In some embodiments, the sample wells are a component of a cartridge (e.g., a disposable or single-use cartridge) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g., a target nucleic acid or a target protein). In some embodiments, the number of molecules within a sample well may be distributed among the sample wells of the sequencing device or module such that some sample wells contain one molecule (e.g., a target nucleic acid or a target protein) while others contain zero, two, or a plurality of molecules.

In some embodiments, a sequencing device or module is positioned to receive a target molecule or sample comprising a plurality of molecules (e.g., a target nucleic acid or a target protein) from a sample preparation device or module. In some embodiments, a sequencing device or module is connected directly (e.g., physically attached to) or indirectly to a sample preparation device or module.

Excitation light is provided to the sequencing device or module from one or more light sources external to the sequencing device or module. Optical components of the sequencing device or module may receive the excitation light from the light source and direct the light towards the array of sample wells of the sequencing device or module and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a nucleic acid or protein (or pluralities thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing device or module may include an optical system for receiving excitation light and directing the excitation light among the sample well array. The optical system may include one or more grating couplers configured to couple excitation light to the sequencing device or module and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device or module by improving the uniformity of excitation light received by sample wells of the sequencing device or module. Examples of suitable components, e.g., for coupling excitation light to a sample well and/or directing emission light to a photodetector, to include in a sequencing device or module are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the sequencing device or module are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device or module, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the sequencing device or module may be used to position and align an excitation source to the sequencing device or module. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference in its entirety. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing device or module may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device or module, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device or module, which may be connected to an instrument interfaced with the sequencing device or module. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device or module. The user interface may be configured to allow a user to input information into the instrument, such as commands and/or settings used to control the functioning of the instrument. In some embodiments, the user interface may include buttons, switches, dials, and/or a microphone for voice commands. The user interface may allow a user to receive feedback on the performance of the instrument and/or sequencing device or module, such as proper alignment and/or information obtained by readout signals from the photodetectors on the sequencing device or module. In some embodiments, the user interface may provide feedback using a speaker to provide audible feedback. In some embodiments, the user interface may include indicator lights and/or a display screen for providing visual feedback to a user.

In some embodiments, the instrument or device described herein may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device or module, and/or data generated from the readout signals of the photodetector.

In some embodiments, the instrument may include a processing device configured to analyze data received from one or more photodetectors of the sequencing device or module and/or transmit control signals to the excitation source(s). In some embodiments, the processing device may comprise a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device or module.

According to some embodiments, the instrument that is configured to analyze target molecules or samples comprising a plurality of molecules based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference in its entirety.

In some embodiments, different numbers of fluorophores of the same type may be linked to different components of a target molecule (e.g., a target nucleic acid or a target protein) or a plurality of molecules present in a sample (e.g., a plurality of nucleic acids or a plurality of proteins), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing nucleic acids or proteins based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” may be used to refer to a peak wavelength within a total bandwidth of radiation output by a source.

### Combined Sample Preparation and Sequencing Device

In some embodiments, a device herein comprising a sample preparation module further comprises a sequencing module. In some embodiments, a device that comprises a sample preparation module and a sequencing module involves a sequencing chip or cartridge that is embedded into a sample preparation cartridge, such that the two cartridges comprise a single, inseparable consumable. In some embodiments, the sequencing chip or cartridge requires consumable support electronics (e.g., a PCB substrate with wirebonds, electrical contacts). The consumable support electronics may be in direct physical contact with the sequencing chip or cartridge. In some embodiments, the sequencing chip or cartridge requires an interface for a peristaltic pump, temperature control and/or electropheresis contacts. These interfaces may allow for precise geometric registration for the many electrical contacts and laser alignment. In some embodiments, different sections of a chip or cartridge may comprise different temperatures, physical forces, electrical interfaces of varying voltage and current, vibration, and/or competing alignment requirements. In some embodiments, disparate instrument sub-systems associated with either the sample preparation or sequencing module must be in close proximity in order to share resources. In some embodiments, a device that comprises a sample preparation module and a sequencing module is hands-free (i.e., can be used without the use of hands).

In some embodiments, a device that comprises a sample preparation module and a sequencing module produces (e.g., enriches or purifies) target nucleic acids with an average read-length for downstream sequencing applications that is longer than an average read-length produced using control methods (e.g., Sage BluePippin methods, manual methods (e.g., manual bead-based size selection methods)). In some embodiments, a sample preparation device produces target nucleic acids with an average read-length for sequencing that comprises at least 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides in length. In some embodiments, a sample preparation device produces target nucleic acids with an average read-length for sequencing that comprises 700-3000, 1000-3000, 1000-2500, 1000-2400, 1000-2300, 1000-2200, 1000-2100, 1000-2000, 1000-1900, 1000-1800, 1000-1700, 1000-1600, 1000-1500, 1000-1400, 1000-1300, 1000-1200, 1500-3000, 1500-2500, 1500-2000, or 2000-3000 nucleotides in length.

In some embodiments, a device that comprises a sample preparation module and a sequencing module allows for shortened times between initiation of sample preparation and detection of a target molecule contained within the sample than control or traditional methods (e.g., Sage BluePippin methods followed by sequencing). In some embodiments, a device that comprises a sample preparation module and a sequencing module is capable of detecting a target molecule using sequencing in less time (e.g., 2-fold, 3-fold, 4-fold, 5-fold, or 10-fold less time) than control or traditional methods (e.g., Sage BluePippin methods followed by sequencing).

In some embodiments, a device that comprises a sample preparation module and a sequencing module is capable of detecting a target molecule with lower inputs of sample than control or traditional methods (e.g., Sage BluePippin methods followed by sequencing). In some embodiments, a device of the disclosure requires as little as 0.1 μg, 0.2 μg, 0.3 μg, 0.4 μg, 0.5 μg, 0.6 μg, 0.7 μg, 0.8 μg, 0.9 μg, or 1 μg of sample (e.g., biological sample). In some embodiments, a device of the disclosure requires as little as 10 μL, 20 μL, 30 μL, 40 μL, 50 μL, 60 μL, 70 μL, 80 μL, 90 μL, 100 μL, 110 μL, 130 μL, 150 μL, 175 μL, 200 μL, 225 μL, or 250 μL of sample (e.g., biological sample such as blood).

### Devices or Modules

In some embodiments, devices or modules (e.g., sample preparation devices; sequencing devices; combined sample preparation and sequencing devices) are configured to transport small volume(s) of fluid precisely with a well-defined fluid flow resolution, and with a well-defined flow rate in some cases. In some embodiments, devices or modules are configured to transport fluid at a flow rate of greater than or equal to 0.1 μL/s, greater than or equal to 0.5 μL/s, greater than or equal to 1 μL/s, greater than or equal to 2 μL/s, greater than or equal to 5 μL/s, or higher. In some embodiments, devices or modules herein are configured to transport fluid at a flow rate of less than or equal to 100 μL/s, less than or equal to 75 μL/s, less than or equal to 50 μL/s, less than or equal to 30 μL/s, less than or equal to 20 μL/s, less than or equal to 15 μL/s, or less. Combinations of these ranges are possible. For example, in some embodiments, devices or modules herein are configured to transport fluid at a flow rate of greater than or equal to 0.1 μL/s and less than or equal to 100 μL/s, or greater than or equal to 5 μL/s and less than or equal to 15 μL/s. For example, in certain embodiments, systems, devices, and modules herein have a fluid flow resolution on the order of tens of microliters or hundreds of microliters. Further description of fluid flow resolution is described elsewhere herein. In certain embodiments, systems, devices, and modules are configured to transport small volumes of fluid through at least a portion of a cartridge.

Some aspects relate to configurations of pumps and apparatuses that include a roller (e.g., in combination with a crank-and-rocker mechanism). Other aspects relate to cartridges comprising channels (e.g., microchannels) having cross-sectional shapes (e.g., substantially triangular shapes), valving, deep sections, and/or surface layers (e.g., flat elastomer membranes). Certain aspects relate to a decoupling of certain components of the peristaltic pump (e.g., the roller) from other components of the pump (e.g., pumping lanes). In some cases, certain elements of apparatuses (e.g., edges of the roller) are configured to interact with elements of the cartridge (e.g., surface layers and certain shapes of the channels) in such a way (e.g., via engagement and disengagement) that any of a variety of advantages are achieved. In some non-limiting embodiments, certain inventive features and configurations of the apparatuses, cartridges, and pumps described herein contribute to improved automation of the fluid pumping process (e.g., due to the use of a translatable roller and a separate cartridge containing multiple different fluidic channels that can be indexed by the roller). In some cases, features described herein contribute to an ability to handle a relatively high number of different fluids (e.g., for multiplexing with multiple samples) with a relatively high number of configurations using a relatively small number of hardware components (e.g., due to the use of separate cartridges with multiple different channels, each of which may be accessible to the roller). As one example, in some cases, the features described herein allow for more than one apparatus to be paired with a cartridge to pump more than one lane simultaneously or use two pumps in one lane for other functionality. In some cases, the features contribute to a reduction in required fluid volume and/or less stringent tolerances in roller/channel interactions (e.g., due to inventive cross-sectional shapes of the channels and/or the edge of the roller, and/or due to the use of inventive valving and/or deep sections of channels). In some cases, features described herein result in a reduction in required washing of hardware components (e.g., due to a decoupling of an apparatus and a cartridge of the peristaltic pump). In some embodiments, aspects of the apparatuses, cartridges, and pumps described herein are useful for preparing samples. For example, some such aspects may be incorporated into a sample preparation module upstream of a detection module (e.g., for analysis/sequencing/identification of biologically-derived samples).

In another aspect, peristaltic pumps are provided. In some embodiments, a peristaltic pump comprises a roller and a cartridge, wherein the cartridge comprises a base layer having a surface comprising channels, wherein at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel. Embodiments of peristaltic pumps are further described elsewhere herein.

In some embodiments, a system (e.g., pump, device) described herein undergoes a pump cycle. In some embodiments, a pump cycle corresponds to one rotation of a crank of the system. In some embodiments, each pump cycle may transport greater than or equal to 1 μL, greater than or equal to 2 μL, greater than or equal to 4 μL, less than or equal to 10 μL, less than or equal to 8 μL, and/or less than or equal to 6 μL of fluid. Combinations of the above-referenced ranges are also possible (e.g., between or equal to 1 μL and 10 μL). Other ranges of volumes of fluid are also possible.

In some embodiments, a system described herein has a particular stroke length. In certain embodiments, given that each pump cycle may transport on the order of between or equal to 1 μL and 10 μL of fluid, and/or given that channel dimensions may preferably be on the order of 1 mm wide and on the order of 1 mm deep (e.g., depending on what can be machined or molded to decrease channel volume and maintain reasonable tolerances), a stroke length may be greater than or equal to 10 mm, greater than or equal to 12 mm, greater than or equal to 14 mm, less than or equal to 20 mm, less than or equal to 18 mm, and/or less than or equal to 16 mm. Combinations of the above-referenced ranges are also possible (e.g., between or equal to 10 mm and 20 mm). Other ranges are also possible. As used herein, “stroke length” refers to a distance a roller travels while engaged with a substrate. In certain embodiments, the substrate comprises a cartridge.

In another aspect, cartridges are provided. In some embodiments, a cartridge comprises a base layer having a surface comprising channels, and at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel. Embodiments of cartridges are further described elsewhere herein.

In some embodiments, a cartridge comprises a base layer. In some embodiments, a base layer has a surface comprising one or more channels. For example, FIG. 8 is a schematic diagram of a cross-section view of a cartridge 100 along the width of channels 102, in accordance with some embodiments. The depicted cartridge 100 includes a base layer 104 having a surface 111 comprising channels 102. In certain embodiments, at least some of the channels are microchannels. For example, in some embodiments, at least some of channels 102 are microchannels. In certain embodiments, all of the channels microchannels. For example, referring again to FIG. 8, in certain embodiments, all of channels 102 are microchannels. As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4A/P, wherein A is a cross-sectional area of the flow of fluid through the channel and P is a wetted perimeter of the cross-section (a perimeter of the cross-section of the channel contacted by the fluid).

In some embodiments, at least a portion of at least some channel(s) have a substantially triangularly-shaped cross-section. In some embodiments, at least a portion of at least some channel(s) have a substantially triangularly-shaped cross-section having a single vertex at a base of the channel and having two other vertices at the surface of the base layer. Referring again to FIG. 24, in some embodiments, at least a portion of at least some of channels 102 have a substantially triangularly-shaped cross-section having a single vertex at a base of the channel and having two other vertices at the surface of the base layer.

As used herein, the term “triangular” is used to refer to a shape in which a triangle can be inscribed or circumscribed to approximate or equal the actual shape, and is not constrained purely to a triangle. For example, a triangular cross-section may comprise a non-zero curvature at one or more portions.

A triangular cross-section may comprise a wedge shape. As used herein, the term “wedge shape” will be known by those of ordinary skill in the art and refers to a shape having a thick end and tapering to a thin end. In some embodiments, a wedge shape has an axis of symmetry from the thick end to the thin end. For example, a wedge shape may have a thick end (e.g., surface opening of a channel) and taper to a thin end (e.g., base of a channel), and may have an axis of symmetry from the thick end to the thin end.

Additionally, in certain embodiments, substantially triangular cross-sections (i.e., “v-groove(s)”) may have a variety of aspect ratios. As used herein, the term “aspect ratio” for a v-groove refers to a height-to-width ratio. For example, in some embodiments, v-groove(s) may have an aspect ratio of less than or equal to 2, less than or equal to 1, or less than or equal to 0.5, and/or greater than or equal to 0.1, greater than or equal to 0.2, or greater than or equal to 0.3. Combinations of the above-referenced ranges are also possible (e.g., between or equal to 0.1 and 2, between or equal to 0.2 and 1). Other ranges are also possible.

In some embodiments, at least a portion of at least some channel(s) have a cross-section comprising a substantially triangular portion and a second portion opening into the substantially triangular portion and extending below the substantially triangular portion relative to the surface of the channel. In some embodiments, the second portion has a diameter (e.g., an average diameter) significantly smaller than an average diameter of the substantially triangular portion. Referring again to FIG. 24, in some embodiments, at least a portion of at least some of channels 102 have a cross-section comprising a substantially triangular portion 101 and a second portion 103 opening into substantially triangular portion 101 and extending below substantially triangular portion 101 relative to surface 105 of the channel, wherein second portion 103 has a diameter 107 significantly smaller than an average diameter 109 of substantially triangular portion 101. In some such cases, the second portion of a channel having a significantly smaller diameter than that of the average diameter of the substantially triangular portion of the channel can result in the substantially triangular portion being accessible to the roller of the apparatus and deformed portions of the surface layer, but the second portion being inaccessible to the roller and deformed portions of the surface layer. For example, referring again to FIG. 24, substantially triangular portion 101 of channel 102 is accessible to a roller (not pictured) and deformed portions of surface layer 106, while second portion 103 is inaccessible to the roller and deformed portions of surface layer 106, in accordance with certain embodiments. In some such cases, a seal with the surface layer 106 cannot be achieved in portions of the channel 102 having a second portion 103, because fluid can still move freely in second portion 103, even when surface layer 106 is deformed by a roller such that it fills substantially triangular portion 101 but not second portion 103. In some embodiments, a portion along a length of a channel may have both a substantially triangular portion and a second portion (“deep section”), while a different portion along the length of the channel has only the substantially triangular portion. In some such embodiments, when the apparatus (e.g., roller) engages with the portion having both a substantially triangular portion and a second portion (deep section), pump action is not started, because a seal with the surface layer is not achieved. However, as the apparatus engages along the length direction of the channel, when the apparatus deforms the surface layer at the portion of the channel having only a substantially triangular section, pump action begins because the lack of second portion (deep section) at that portion allows for a seal (and consequently a pressure differential) to be created. Therefore, in some cases, the presence and absence of deep sections along the length of the channels of the cartridge can allow for control of which portions of the channel are capable of undergoing pump action upon engagement with the apparatus.

The inclusion of such “deep sections” as second portions of at least some of the channels of the cartridge may contribute to any of a variety of potential benefits. For example, such deep sections (e.g., second portion 103) may, in some cases, contribute to a reduction in pump volume in peristaltic pumping processes. In some such cases, pump volume can be reduced by a factor of two or more for higher volume resolution. In some cases, such deep sections may also provide for a well-defined starting point for the pump volume that is not determined by where the roller lands on the channel. For example, the interface between a portion of a channel having both a substantially triangular portion and a second portion (deep section) and a portion of a channel having only a substantially triangular portion can, in some cases, be used as a well-defined starting point for the pump volume, because only fluid occupying the volume of the latter channel portion can be pumped. In some cases, where the rollers lands on the channel may have some error associated depending on any of a variety of factors, such as cartridge registration. The inclusion of deep sections may, in some cases, reduce or eliminate variations in pump volume associated with such error.

As used herein, an average diameter of a substantially triangular portion of a channel may be measured as an average over the z-axis from the vertex of the substantially triangular portion to the surface of the channel.

### SCODA

SCODA can involve providing a time-varying driving field component that applies forces to particles in some medium in combination with a time-varying mobility-altering field component that affects the mobility of the particles in the medium. The mobility-altering field component is correlated with the driving field component so as to provide a time-averaged net motion of the particles. SCODA may be applied to cause selected particles to move toward a focus area.

In one embodiment of SCODA based purification, described herein as electrophoretic SCODA, time varying electric fields both provide a periodic driving force and alter the drag (or equivalently the mobility) of molecules that have a mobility in the medium that depends on electric field strength, e.g. nucleic acid molecules. For example, DNA molecules have a mobility that depends on the magnitude of an applied electric field while migrating through a sieving matrix such as agarose or polyacrylamide. By applying an appropriate periodic electric field pattern to a separation matrix (e.g. an agarose or polyacrylamide gel) a convergent velocity field can be generated for all molecules in the gel whose mobility depends on electric field. The field dependent mobility is a result of the interaction between a repeating DNA molecule and the sieving matrix, and is a general feature of charged molecules with high conformational entropy and high charge to mass ratios moving through sieving matrices. Since nucleic acids tend to be the only molecules present in most biological samples that have both a high conformational entropy and a high charge to mass ratio, electrophoretic SCODA based purification has been shown to be highly selective for nucleic acids.

The ability to detect specific biomolecules in a sample has wide application in the field of diagnosing and treating disease. Research continues to reveal a number of biomarkers that are associated with various disorders. Exemplary biomarkers include genetic mutations, the presence or absence of a specific protein, the elevated or reduced expression of a specific protein, elevated or reduced levels of a specific RNA, the presence of modified biomolecules, and the like. Biomarkers and methods for detecting biomarkers are potentially useful in the diagnosis, prognosis, and monitoring the treatment of various disorders, including cancer, disease, infection, organ failure and the like.

The differential modification of biomolecules in vivo is an important feature of many biological processes, including development and disease progression. One example of differential modification is DNA methylation. DNA methylation involves the addition of a methyl group to a nucleic acid. For example a methyl group may be added at the 5′ position on the pyrimidine ring in cytosine. Methylation of cytosine in CpG islands is commonly used in eukaryotes for long term regulation of gene expression. Aberrant methylation patterns have been implicated in many human diseases including cancer. DNA can also be methylated at the 6 nitrogen of the adenine purine ring.

Chemical modification of molecules, for example by methylation, acetylation or other chemical alteration, may alter the binding affinity of a target molecule and an agent that binds the target molecule. For example, methylation of cytosine residues increases the binding energy of hybridization relative to unmethylated duplexes. The effect is small. Previous studies report an increase in duplex melting temperature of around 0.7° C. per methylation site in a 16 nucleotide sequence when comparing duplexes with both strands unmethylated to duplexes with both strands methylated.

Affinity SCODA

SCODAphoresis is a method for injecting biomolecules into a gel, and preferentially concentrating nucleic acids or other biomolecules of interest in the center of the gel. SCODA may be applied, for example, to DNA, RNA and other molecules. Following concentration, the purified molecules may be removed for further analysis. In one specific embodiment of SCODAphoresis-affinity SCODA-binding sites which are specific to the biomolecules of interest may be immobilized in the gel. In doing so one may be able generate a non-linear motive response to an electric field for biomolecules that bind to the specific binding sites. One specific application of affinity SCODA is sequence-specific SCODA. Here oligonucleotides may be immobilized in the gel allowing for the concentration of only DNA molecules which are complementary to the bound oligonucleotides. All other DNA molecules which are not complementary may focus weakly or not at all and can therefore be washed off the gel by the application of a small DC bias.

SCODA based transport is a general technique for moving particles through a medium by first applying a time-varying forcing (i.e. driving) field to induce periodic motion of the particles and superimposing on this forcing field a time-varying perturbing field that periodically alters the drag (or equivalently the mobility) of the particles (i.e. a mobility-altering field). Application of the mobility-altering field is coordinated with application of the forcing field such that the particles will move further during one part of the forcing cycle than in other parts of the forcing cycle.

By varying the drag (i.e. mobility) of the particle at the same frequency as the external applied force, a net drift can be induced with zero time-averaged forcing. An appropriate choice of driving force and drag coefficients that vary in time and space can generate a convergent velocity field in one or two dimensions. A time varying drag coefficient and driving force can be utilized in a real system to specifically concentrate (i.e. preferentially focus) only certain molecules, even where the differences between the target molecule and one or more non-target molecules are very small, e.g. molecules that are differentially modified at one or more locations, or nucleic acids differing in sequence at one or more bases.

An affinity matrix can be generated by immobilizing an agent with a binding affinity to the target molecule (i.e. a probe) in a medium. Using such a matrix, operating conditions can be selected where the target molecules transiently bind to the affinity matrix with the effect of reducing the overall mobility of the target molecule as it migrates through the affinity matrix. The strength of these transient interactions is varied over time, which has the effect of altering the mobility of the target molecule of interest. SCODA drift can therefore be generated. This technique is called affinity SCODA, and is generally applicable to any target molecule that has an affinity to a matrix.

Affinity SCODA can selectively enrich for nucleic acids based on sequence content, with single nucleotide resolution. In addition, affinity SCODA can lead to different values of k for molecules with identical DNA sequences but subtly different chemical modifications such as methylation. Affinity SCODA can therefore be used to enrich for (i.e. preferentially focus) molecules that differ subtly in binding energy to a given probe, and specifically can be used to enrich for methylated, unmethylated, hypermethylated, or hypomethylated sequences.

Exemplary media that can be used to carry out affinity SCODA include any medium through which the molecules of interest can move, and in which an affinity agent can be immobilized to provide an affinity matrix. In some embodiments, polymeric gels including polyacrylamide gels, agarose gels, and the like are used. In some embodiments, microfabricated/microfluidic matrices are used.

Exemplary operating conditions that can be varied to provide a mobility altering field include temperature, pH, salinity, concentration of denaturants, concentration of catalysts, application of an electric field to physically pull duplexes apart, or the like.

Exemplary affinity agents that can be immobilized on the matrix to provide an affinity matrix include nucleic acids having a sequence complementary to a nucleic acid sequence of interest, proteins having different binding affinities for differentially modified molecules, antibodies specific for modified or unmodified molecules, nucleic acid aptamers specific for modified or unmodified molecules, other molecules or chemical agents that preferentially bind to modified or unmodified molecules, or the like.

The affinity agent may be immobilized within the medium in any suitable manner. For example where the affinity agent is an oligonucleotide, the oligonucleotide may be covalently bound to the medium, acrydite modified oligonucleotides may be incorporated directly into a polyacrylamide gel, the oligonucleotide may be covalently bound to a bead or other construct that is physically entrained within the medium, or the like.

Where the affinity agent is a protein or antibody, in some embodiments the protein may be physically entrained within the medium (e.g. the protein may be cast directly into an agarose or polyacrylamide gel), covalently coupled to the medium (e.g. through use of cyanogen bromide to couple the protein to an agarose gel), covalently coupled to a bead that is entrained within the medium, bound to a second affinity agent that is directly coupled to the medium or to beads entrained within the medium (e.g. a hexahistidine tag bound to NTA-agarose), or the like.

Where the affinity agent is a protein, the conditions under which the affinity matrix is prepared and the conditions under which the sample is loaded should be controlled so as not to denature the protein (e.g. the temperature should be maintained below a level that would be likely to denature the protein, and the concentration of any denaturing agents in the sample or in the buffer used to prepare the medium or conduct SCODA focusing should be maintained below a level that would be likely to denature the protein).

Where the affinity agent is a small molecule that interacts with the molecule of interest, the affinity agent may be covalently coupled to the medium in any suitable manner.

One embodiment of affinity SCODA is sequence-specific SCODA. In sequence specific SCODA, the target molecule is or comprises a nucleic acid molecule having a specific sequence, and the affinity matrix contains immobilized oligonucleotide probes that are complementary to the target nucleic acid molecule. In some embodiments, sequence specific SCODA is used both to separate a specific nucleic acid sequence from a sample, and to separate and/or detect whether that specific nucleic acid sequence is differentially modified within the sample. In some such embodiments, affinity SCODA is conducted under conditions such that both the nucleic acid sequence and the differentially modified nucleic acid sequence are concentrated by the application of SCODA fields. Contaminating molecules, including nucleic acids having undesired sequences, can be washed out of the affinity matrix during SCODA focusing. A washing bias can then be applied in conjunction with SCODA focusing fields to separate the differentially modified nucleic acid molecules as described below by preferentially focusing the molecule with a higher binding energy to the immobilized oligonucleotide probe.

### EXAMPLES

Embodiments of the invention are further described with reference to the following examples, which are intended to be illustrative and not restrictive in nature.

### Example 1—Use of a Sample Preparation Device

An automated sample preparation device of the disclosure was used to prepare a sample of DNA extracted from human blood.

The sample preparation device comprised a fluidics module (comprising a peristaltic pumping system), a temperature control module (to provide temperature and mechanical precision), a touch screen interface on the device that allowed the user to select any process-specific parameters (e.g., range of desired size of the nucleic acids, desired degree of homology for target molecule capture, etc.), and a lid that the user was able open in order to insert a sample preparation cartridge of the disclosure. The device was powered with a 1000-volt electrode supply. The sample preparation cartridge comprised thirteen discrete microfluidics channels (or pumping lanes) and was fabricated such that it could perform end-to-end sample preparation. The microfluidic channels were designed to manipulate reagents and the cartridge enabled, in automated succession: (1) Pipet introduction of combined sample lysis using lysis+Lysis buffer and subsequent extraction of target DNA; (2) DNA purification; (3) DNA tagmentation using transposase Tn5 succeeded by DNA repair; (4) selection of DNA fragments of particular size range using nucleic acid capture probes and SCODA; and (5) DNA clean-up. 100 μL of whole human blood was mixed with lysis buffer and Proteinase K was incubated at 55° C. for 10 minutes then mixed with isopropanol; lysate mixture was subsequently added to a sample port in the sample preparation cartridge, the loaded cartridge was inserted into the sample preparation device, and DNA was extracted. The automated device, as described above, yielded 1.2 μg extracted DNA; 1 μg of that extracted DNA was further processed using the successive steps described above to generate 530 ng of a DNA library at a concentration of 6.5 nM. This purified DNA library produced by the sample preparation device was then subjected to sequencing using a glass sequencing chip.

As a control experiment, 100 μL of whole human blood (from the same sample as above) was manually processed to generate DNA library for sequencing using traditional DNA extraction and purification techniques.

The inventors found that sequencing data acquired using DNA library prepared using the automated sample preparation device was similar in quality (e.g., as assessed by average read length) relative to the sequencing data acquired using DNA manually prepared using traditional DNA extraction and purification techniques. As shown in Table 3, the automated device generated more total reads (72 total reads using automated process compared to 27 total reads using manual process) and greater read lengths (1989.0±760.1 base pair read lengths using automated process compared to 1132.1±324.5 base pair read lengths using manual process) than the manual process, with no significant difference observed between the processes in terms of accuracy and GC content of the resulting reads.

TABLE 3
Sequencing results from DNA libraries generated from whole human blood

| | | | Standard | | Standard | | |
|---|---|---|---|---|---|---|---|
| | | Average | Deviation | Average | Deviation | | Standard |
| | | Read | Read | Read | Read | Average | Deviation |
| | Total | Length | Length | Accuracy | Accuracy | GC content | GC content |
| | Reads | (bp) | (bp) | (%) | (%) | (%) | (%) |
| Manual process | 27 | 1132.1 | 324.5 | 60.7% | 4.1% | 35.2% | 4.5% |
| Automated process | 72 | 1989.0 | 760.1 | 59.9% | 4.3% | 37.0% | 4.7% |
| using Sample | | | | | | | |
| Preparation | | | | | | | |
| device of this | | | | | | | |
| disclosure | | | | | | | |

### Example 2—Use of a Sample Preparation Device to Enrich DNA for Sequencing

An automated sample preparation device of the disclosure was used to prepare a sample of DNA extracted from cultured E. coli cells.

The sample preparation device comprised a fluidics module (comprising a peristaltic pumping system), a temperature control module (to provide temperature and mechanical precision), a touch screen interface on the device that allowed the user to select any process-specific parameters (e.g., range of desired size of the nucleic acids, desired degree of homology for target molecule capture, etc.), and a lid that the user was able open in order to insert a sample preparation cartridge of the disclosure. The device was powered with a 1000-volt electrode supply. The sample preparation cartridge comprised thirteen discrete microfluidics channels (or pumping lanes) and was fabricated such that it could perform end-to-end sample preparation. The microfluidic channels were designed to manipulate reagents and the cartridge enabled, in automated succession: (1) Pipet introduction of combined sample+Lysis buffer and subsequent extraction of target DNA; (2) DNA purification; (3) DNA tagmentation using transposase Tn5 succeeded by DNA repair; (4) selection of DNA fragments of particular size range using SCODA; and (5) DNA clean-up.

A sample of seven-hundred million E. coli cells from an overnight culture mixed with lysis buffer and Proteinase K was incubated at 55° C. for 10 minutes then mixed with isopropanol; lysate mixture was added to a sample port in the sample preparation cartridge, the loaded cartridge was inserted into the sample preparation device, and DNA was extracted. Automated processing continued to render the DNA into DNA library ready for sequencing with a brief pause for the user to add DNA Repair Enzyme and DNA Repair Buffer Mix to the cartridge just prior to the DNA Repair step. The automated device transported the DNA Repair Enzyme and DNA Repair Buffer Mix to the reaction location in the cartridge. The automated device, as described above, yielded 0.96 μg extracted DNA; subsequent automated steps generated 279 ng of a DNA library at a concentration of 2.89 nM.

As a control experiment, a sample of seven-hundred million E. coli cells (from the same sample as above) was manually processed to generate DNA using traditional DNA extraction and purification techniques. This manually prepared DNA was subjected to the same automated library preparation process on the automated device generating 199 ng of a DNA library at a concentration of 2.65 nM.

The purified DNA libraries produced by the sample preparation device were concentrated using Aline beads and then subjected to sequencing on a Pacific Biosciences® RSII DNA Sequencer.

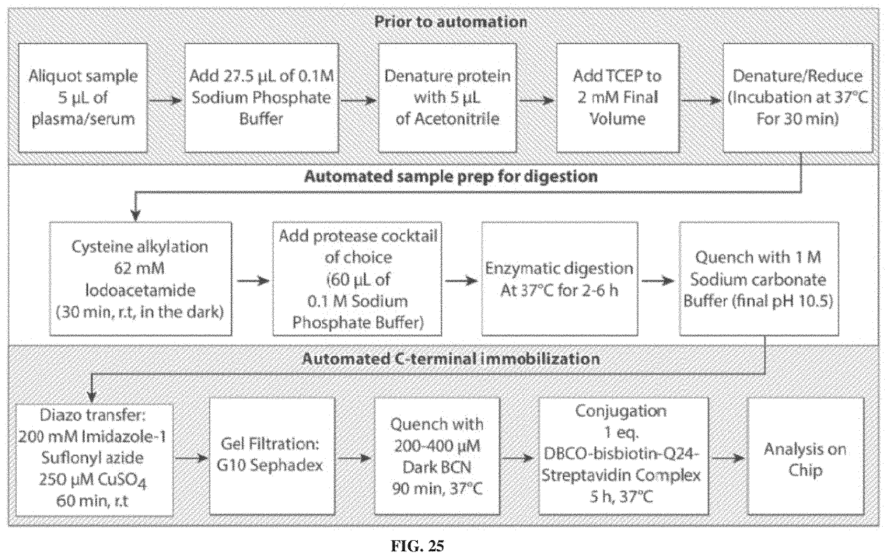

The inventors found that sequencing data acquired using DNA purified and prepared into library format using the automated sample preparation device generated sequencing reads that were slightly shorter in length, but similar in quality (as assessed by Rsq score) relative to the sequencing data acquired using DNA manually prepared with traditional DNA extraction and purification techniques followed by automated DNA library preparation (FIG. 25).

As shown in Table 4, the fully automated library generated reads with identical read quality (Rsq 0.82) to those generated with manual DNA extraction, with roughly equivalent read lengths (851 base average reads lengths versus 922 for manual).

TABLE 4
Sequencing results from DNA libraries generated from E. coli cells extracted
and purified via an Automated Sample Preparation Device versus manually
extracted and purified DNA run on the same automated device.

| | | | | Median | |
|---|---|---|---|---|---|
| Seq | | | | read | |
| name | Library | Treatment | Reads | length | RSq |
| C1856 | E2E | From lysate, E. coli library | 5756 | 851 | 0.82 |
| | | (Sample Prep device of this disclosure) | | | |
| C890 | MEAL | From purified DNA, E. coli library | 7674 | 922 | 0.82 |
| | | (Sample Prep device of this disclosure) | | | |

### Example 3—Use of a Sample Preparation Device to Enrich DNA for Sequencing

An automated sample preparation device of the disclosure was used to select DNA fragments of a particular size range using SCODA for a DNA library manually prepared from E. coli cultured cells.

Four micrograms of manually purified E. coli DNA was subjected to Tn5a tagmentation and then split into four separate samples consisting of 1 μg each. Selection of DNA fragments of a particular size was conducted separately by four different methods (1) Sage BluePippin with program to collect fragments from 3 kb to 10 kb in size, (2) Sage BluePippin with program to collect fragments greater in size than 4 kb to 10 kb, (3) manual Aline bead size selection with 0.45× bead addition, or (4) SCODA technology as in the automated sample preparation device (described in Example 8.0).

After size selection, each sample was separately prepared into DNA library and sequenced on a Pacific Biosciences® RSII DNA Sequencer.

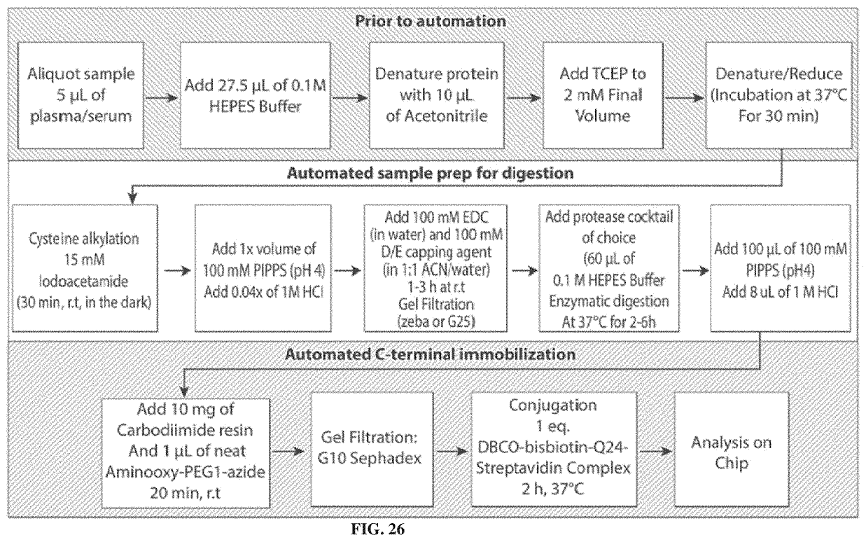

The inventors found that sequencing data acquired using DNA library size selection using the automated sample preparation device was superior to or equivalent to replicate DNA libraries selected for size by the standard manual bead-based process or the automated Sage BluePippin size selection method (FIG. 26).

As shown in Table 5 (below), the automated device generated read lengths longer than the manual size selection process and equivalent to the BluePippin methods with no significant difference observed among the processes in terms of accuracy and GC content of the resulting reads.

TABLE 5
Sequencing metrics from DNA libraries generated automated
size selection compared to those derived from samples
size selected by commercial and manual methods

| | | Median |
|---|---|---|
| | | read |
| Size selection | Reads | length |
| Sage BluePippin, selecting for 3-10 kb range | 675 | 2389 |
| Sage BluePippin, selecting >4-10 kb high pass | 2253 | 2409 |
| Manual bead-based size selection (Aline) | 2296 | 1478 |
| Automated size selection | 18707 | 2358 |
| (Sample Prep device of this disclosure) | | |

### Example 4—Preparation of a Biological Sample for Sequencing

Sample Lysis

Cultured cells or tissue samples comprising one or more target molecules (e.g., proteins) are lysed using any method known to a skilled person. The biological samples are suspended in lysis buffer (e.g., RIPA buffer, GCl (Guanidine-HCl) buffer, GlyNP40 buffer) and mechanically homogenized to break down cell walls (e.g., in a lysis cartridge). Once the cells are disrupted, the target molecules are then precipitated and the supernatant discarded. Precipitation can be accomplished using centrifugation including washing steps (e.g., addition of either a mix of chloroform/methanol or trichloroacetic acid). See FIG. 3.

Enrichment

The lysed sample is then optionally enriched (e.g., using affinity matrices) to capture the target molecules and discard the remaining non-target molecules (e.g., in an enrichment cartridge). Enrichment may include depletion strategies utilized to reduce sample complexity by sequestering the non-target molecules (e.g., using affinity matrices). See FIG. 4.

Fragmentation

The lysed sample (if not enriched) or the enriched sample may then be fragmented (e.g., digested) (e.g., in a fragmentation cartridge). This step in the sample process converts target molecules into smaller fragments or subunits. This step can be conducted using non-enzymatic and/or enzymatic processes. Non-enzymatic methods include (but are not limited to) acid hydrolysis, cleavage via cyanogen bromide, hydroxylamine, and 2-nitro-5-thiocyanobenzoic acid, and electrochemical oxidation. Enzymatic methods include (but are not limited to) the use of nucleases or proteases. See FIG. 6.

Functionalization

Prior to sequencing, the fragmented sample may be functionalized at one of its terminal moieties (e.g., N-terminus or C-terminus of a protein fragment) (e.g., in a functionalization cartridge). For example, digested peptides may be labeled with some moiety capable of immobilizing the peptides on the sequencing substrate. Functionalization can be accomplished through a variety of chemical or enzymatic methods. See FIGS. 6 and 7.

### Example 5—Preparation of a Protein Sample

This example describes the preparation of a protein sample using a device of the disclosure, wherein the incubation, functionalization, quenching, immobilization complex forming, and purifying steps were performed on a single cartridge. Proteins were prepared by pulldown from spiked plasma, wherein the enriched protein was purified using either an antibody or a DNA aptamer on a solid support. Proteins were then equilibrated with the desired buffer, either by gel filtration or by pH adjustment. Then, an enriched protein sample (50-200 μM in 100 μL) comprising an equal mixture of 2, 3, or 4 proteins was prepared in 100 mM HEPES or sodium phosphate (pH 6-9) with 10-20% acetonitrile was mixed with a solution of tris(2-carboxyethyl)phosphine hydrochloride (TCEP-HCl, 200 mM in water, 1 μL), to act as a reducing agent, freshly dissolved iodoacetamide solution (9 mg in 97.3 μL water for 500 mM, 2 μL), to act as an amino acid side-chain capping agent, and Trypsin (1 μg/μL, 0.5-1 μL), to act as a protein digestion agent. Next, the peptide sample was incubated at 37° C. for 6 to 10 hours in the digestion portion, wherein the protein was denatured and digested. This resulted in the formation of a digested peptide sample.

Next, the digested peptide sample was automatedly transported through a series of reservoirs, where it mixed with a functionalization agent, a first (catalytic) reagent, and a second (pH-adjusting) reagent. Initially, the digested peptide sample was automatedly added to potassium carbonate (1 M, 5 μL), to adjust the pH to a value of 10-11. Following this, the digested peptide sample was automatedly exposed to imidazole-1-sulfonyl azide solution (“ISA” 200 mM in 200 mM KOH, 1.2 μL), an azide transfer agent. Next, the digested peptide sample was automatedly mixed with copper sulfate (a catalytic reagent) solution. Finally, the digested peptide sample was automatedly transferred to a functionalization portion of the modular cartridge where was incubated for one hour at room temperature. This resulted in the formation unquenched mixture comprising one or more derivatized peptides.

Following functionalization of the peptides in the functionalization region, 50 μL of the unquenched sample was automatedly transported to a portion of the of the modular cartridge where it was mixed with a plurality of polystyrene beads (a solid substrate), and quenched using 10 actively mixed quench steps, with each quench step followed by a stationary mixing step, for a total of 23 minutes. Finally, the resulting quenched mixture was passed through an on-cartridge column to filter it from the plurality of polystyrene beads.

Next, the pH of the quenched peptide sample was adjusted to between 7 and 8 through the addition of 6 μL of 1 M acetic acid. Following this, the quenched mixture was automatedly mixed with DBCO-Q24-SV (50 μM, 6 μL), an immobilization complex, before being incubated at 37° C. on the device for 4 hours. Following this, the peptide sample was automatedly transported to a column of the modular cartridge, consisting of Zeba de-salting column resin with a cut off of 40 kDa that was equilibrated first with 10 mM TRIS, 10 mM potassium acetate buffer (pH 7.5). Finally, the purified peptide sample that resulted from this workflow was frozen and stored at a temperature below −20° C.

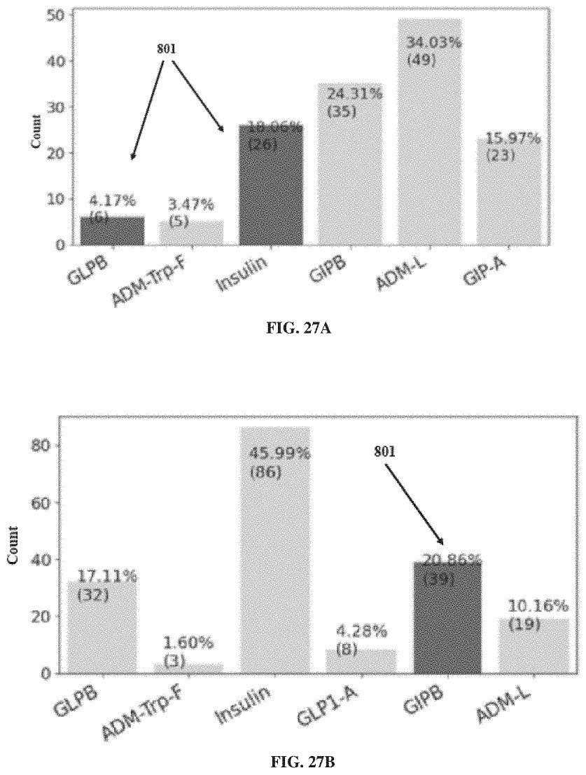

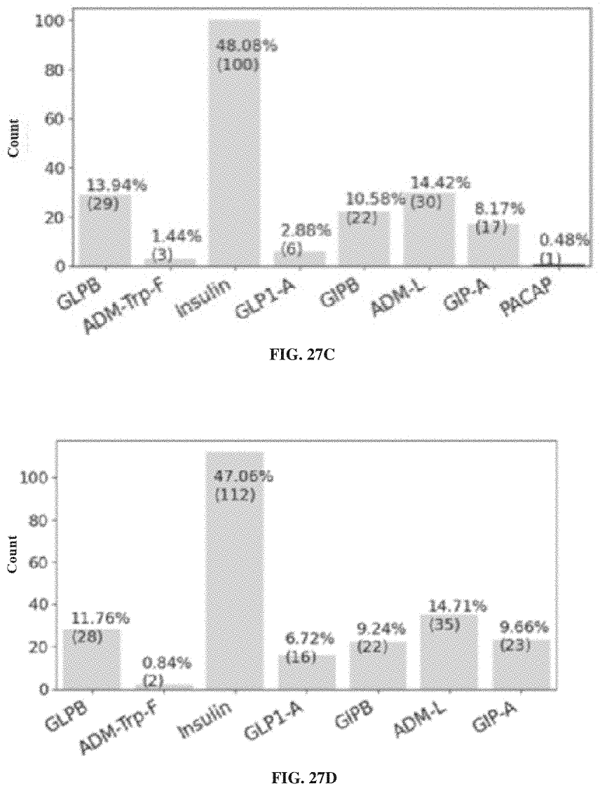

At a later time, purified peptide samples were sequenced, and observed peptides were identified based on their correspondence to protein sequences. FIGS. 27A-27D present the results in the form of bar charts. FIG. 27A corresponds to a mixture of two proteins—GIP and ADM. FIG. 27B corresponds to a mixture of three proteins—GLP1, Insulin, and ADM. FIG. 27C corresponds to a mixture of four proteins—GLP1, ADM, Insulin, and GIP. FIG. 27D corresponds to a mixture of four peptides—GLP1, ADM, Insulin, and GIP. A few off-target assignments 801 are indicated, but in general the peptides sequenced were correctly assigned to the proteins prepared in the peptide sample. Moreover, the generated libraries in this example had similar or more total reads than replicate manually prepared libraries of the same protein mixes. This example demonstrates that a purified peptide sample can be prepared in an automated way on a modular cartridge of the type disclosed here.

### Example 6—Use of a Device of the Disclosure

This example describes an exemplary device, wherein the incubation, functionalization, quenching, immobilization complex forming, and purifying steps may be performed using a device of the disclosure comprising multiple modular cartridges. Although the modular cartridges of this embodiment are not connected, peptide samples were prepared by following the protocol of Example 5. The protein sample was loaded and then incubated (e.g. at 37° C. for 5 hours), wherein the protein was denatured and digested. The cartridges further comprised pump lanes to facilitate pumping of the fluids within the cartridge, as well as a reagent/sample mixture source.

After incubation, the peptide sample became a digested peptide sample. The digested peptide sample was then automatedly transferred to a second cartridge, where it was automatedly transported through a series of reservoirs, where it mixed with a functionalization agent, a first (catalytic) reagent, and a second (pH-adjusting) reagent. The digested peptide sample was transported to the second cartridge through a sample input. The digested peptide sample was automatedly transported mixed with the functionalization agent, a first (catalytic) reagent, and a second (pH-adjusting) reagent, in sequence. Finally, the digested peptide sample was incubated for the period of time (e.g. one hour at room temperature). This resulted in the formation of an unquenched mixture. The second cartridge further comprised pump lanes.

A portion of the unquenched sample was automatedly transported to a third cartridge comprising a sample input, a filter for beads, a small volume acidic reagent reservoir, and mixing channels. Here, the unquenched mixture was quenched at room temperature. Finally, the resulting quenched mixture was passed through an on-cartridge column to remove the plurality of polystyrene beads, and the pH was adjusted to between 7 and 8 by the addition of acetic acid from an acidic reagent reservoir.

Following this, the quenched mixture was mixed with the DBCO-Q24-SV immobilization complex in the mixture source of the first modular cartridge, before it was incubated at 37° C.

Finally, the peptide sample was automatedly transported to a fourth cartridge, which controlled the flow of the quenched peptide sample through a commercial Zeba de-salting column resin. Additional equilibration buffer was dispensed through the column to ensure that the peptides were transmitted through the column. The purified peptide sample was collected from a specific fraction of the fluid passing through the column, while the remaining fluid was transmitted to a waste reservoir. This example demonstrates that in some embodiments, purified peptide samples can be produced automatedly using devices comprising multiple cartridges.

### Additional Embodiments

Additional embodiments of the present disclosure are encompassed by the following numbered paragraphs:

1. A device for preparing a biological sample for sequencing, wherein the device comprises an automated module configured to receive (iii) a fragmentation cartridge comprising one or more microfluidic channels and configured to digest or fragment at least one of the one or more target molecules to produce a fragmented sample; and one or more of the cartridges selected from (i) a lysis cartridge (ii) an enrichment cartridge, and (iv) a functionalization cartridge;

wherein (i), (ii), and (iv) are defined as follows:

(i) a lysis cartridge comprises one or more microfluidic channels and configured to intake a biological sample comprising one or more target molecules and produce a lysed sample;

(ii) an enrichment cartridge comprises one or more microfluidic channels and is configured to enrich at least one of the one or more target molecules to produce an enriched sample; and

(iv) a functionalization cartridge comprises one or more microfluidic channels and is configured to functionalize a terminal moiety of at least one of the one or more target molecules to produce a functionalized sample.

2. The device of paragraph 1, wherein the biological sample is a single cell, mammalian cell tissue, animal sample, fungal sample, or plant sample.

3. The device of paragraph 1, wherein the biological sample is a blood sample, saliva sample, sputum sample, fecal sample, urine sample, buccal swab sample, amniotic sample, seminal sample, synovial sample, spinal sample, or pleural fluid sample.

4. The device of any one of paragraphs 1-3, wherein the one or more target molecules are nucleic acids.

5. The device of paragraph 1 or 3, wherein the one or more target molecules are proteins.

6. The device of any one of paragraphs 1-5, wherein the one or more microfluidic channels are configured to contain and/or transport fluid(s) and/or reagent(s).

7. The device of any one of paragraphs 1-6, wherein the fragmentation cartridge comprises non-enzymatic reagents that digest or fragment the sample and/or the one or more target molecules.

8. The device of paragraph 7, wherein the non-enzymatic reagents that digest or fragment the sample and/or the one or more target molecules comprise detergents, acids, and/or bases.

9. The device of paragraph 7 or 8, wherein the non-enzymatic reagents that digest or fragment the sample and/or the one or more target molecules comprise cyanogen bromide, hydroxylamine, iodosobenzoic acid, dimethyl sulfoxide, hydrochloric acid, BNPS-skatole [2-(2-nitrophenylsulfenyl)-3-methylindole], and/or 2-nitro-5-thiocyanobenzoic acid.

10. The device of any one of paragraphs 1-6, wherein the fragmentation cartridge comprises one or more enzymatic reagents that digest or fragment at least one of the one or more target molecules.

11. The device of paragraph 10, wherein the one or more enzymatic reagents comprise one or more proteases.

12. The device of paragraph 11, wherein the one or more proteases are selected from the group consisting of: trypsin, chymotrypsin, LysC, LysN, AspN, GluC and ArgC.

13. The device of paragraph 10, wherein the one or more enzymatic reagents comprise one or more endonucleases or exonucleases.

14. The device of any one of paragraphs 1-13, wherein the fragmentation cartridge can be heated at an elevated temperature (e.g., 20-60° C.).

15. The device of any one of paragraphs 1-14, wherein the device is configured to heat the fragmentation cartridge at an elevated temperature (e.g., 20-60° C.).

16. The device of any one of paragraphs 1-15, wherein the device is configured to subject the fragmentation cartridge to microwaves or sonication.

17. The device of any one of paragraphs 1-16, wherein the module is further configured to receive a lysis cartridge.

18. The device of paragraph 1, wherein the fragmentation cartridge is positioned to receive the lysed sample from the lysis cartridge.

19. The device of paragraph 1 or 18, wherein the lysis cartridge and the fragmentation cartridge are connected by one or more microfluidic channels.

20. The device of paragraph 1, wherein the lysed sample can be removed from the device (e.g. to enable manual enrichment).

21. The device of any one of paragraphs 1-20, wherein the device is configured such that the lysed sample is enriched prior to fragmentation.

22. The device of any one of paragraphs 17-21, wherein the lysis cartridge comprises reagents that lyse the sample but does not degrade or fragment the one or more target molecules.

23. The device of any one of paragraphs 17-22, wherein the lysis cartridge comprises reagents that promote the one or more target molecules to be at least partially isolated or purified from non-target molecules of the sample.

24. The device of paragraph 22 or 23, wherein the reagents comprise detergents, acids, and/or bases.

25. The device of any one of paragraphs 22-24 wherein the reagents comprise a lysis buffer.

26. The device of paragraph 25, wherein the lysis buffer is selected from the group consisting of: RIPA buffer, GCl (Guanidine-HCl) buffer, and GlyNP40 buffer.

27. The device of any one of paragraphs 17-26, wherein the one or more microfluidic channels in the lysis cartridge promote shearing of cells and/or tissues (e.g., shear flow of cells and/or tissues).

28. The device of any one of paragraphs 17-26, wherein the lysis cartridge comprises a needle passage that promotes mechanical shearing of cells and/or tissues.

29. The device of paragraph 28, wherein the needle passage has an internal diameter of 0.1 to 1 mm.

30. The device of any one of paragraphs 17-29, wherein the one or more microfluidic channels in the lysis cartridge comprise a post array.

31. The device of any one of paragraphs 17-30, wherein the lysis cartridge is configured to be heated at an elevated temperature (e.g., 20-60° C.).

32. The device of any one of paragraphs 17-31, wherein the device is configured to heat the lysis cartridge at an elevated temperature (e.g., 20-60° C.).

33. The device of any one of paragraphs 17-32, wherein the device is configured to subject the lysis cartridge to microwaves or sonication.

34. The device of any one of paragraphs 1-33, wherein the module is further configured to receive an enrichment cartridge.

35. The device of paragraph 34, wherein the enrichment cartridge is positioned to receive the lysed sample from the lysis cartridge.

36. The device of paragraph 34 or 35, wherein the lysis cartridge and the enrichment cartridge are connected by one or more microfluidic channels.

37. The device of paragraph 34, wherein the fragmentation cartridge is positioned to receive the enriched sample from the enrichment cartridge.

38. The device of paragraph 37, wherein the enrichment cartridge and the fragmentation cartridge are connected by one or more microfluidic channels.

39. The device of any one of paragraphs 1-38, wherein the enrichment cartridge comprises one or more affinity matrices.

40. The device of paragraph 39, wherein the one or more affinity matrices are in microfluidic channels of the enrichment cartridge.

41. The device of paragraph 39 or 40, wherein the one or more target molecules are nucleic acids, wherein the immobilized capture probe is an oligonucleotide capture probe, and wherein the oligonucleotide capture probe comprises a sequence that is at least partially complementary to at least one of the one or more target molecules.

42. The device of paragraph 41, wherein the oligonucleotide capture probe comprises a sequence that is at least 80%, 90% 95%, or 100% complementary to the target molecule.

43. The device of any one of paragraphs 39-42, wherein the device produces nucleic acids with an average read-length that is longer than an average read-length produced using control methods.

44. The device of paragraph 39, wherein the one or more target molecules are proteins, and wherein the immobilized capture probe is a protein capture probe that binds to at least one of the one or more target molecules.

45. The device of paragraph 44, wherein the protein capture probe is an aptamer or an antibody.

46. The device of paragraph 44 or 45, wherein the protein capture probe binds to the target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M.

47. The device of paragraph 39, wherein the one or more target molecules are nucleic acids, wherein the immobilized capture probe is an oligonucleotide capture probe, and wherein the oligonucleotide capture probe comprises a sequence that is at least partially complementary to at least one non-target molecule.

48. The device of paragraph 47, wherein the oligonucleotide capture probe comprises a sequence that is at least 80%, 90% 95%, or 100% complementary to the non-target molecule.

49. The device of paragraph 47 or 48, wherein the oligonucleotide capture probe is not complementary to the one or more target molecules.

50. The device of paragraph 39, wherein the one or more target molecules are proteins, and wherein the immobilized capture probe is a protein capture probe that binds to at least one non-target molecule.

51. The device of paragraph 50, wherein the protein capture probe is an aptamer or an antibody.

52. The device of paragraph 50 or 51, wherein the protein capture probe binds to the non-target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M.

53. The device of any one of paragraphs 50-52, wherein the protein capture probe does not bind to the one or more target molecules.

54. The device of any one of paragraphs 47-53, wherein the enrichment cartridge is configured to deplete the sample of non-target molecules.

55. The device of any one of paragraphs 1-54, wherein the module is further configured to receive a functionalization cartridge.

56. The device of paragraph 55, wherein the lysis cartridge and the functionalization cartridge are connected by one or more microfluidic channels.

57. The device of paragraph 55, wherein the enrichment cartridge and the functionalization cartridge are connected by one or more microfluidic channels.

58. The device of paragraph 55, wherein the fragmentation cartridge and the functionalization cartridge are connected by one or more microfluidic channels.

59. The device of paragraph 58, wherein the functionalization cartridge is positioned to receive the fragmented sample from the fragmentation cartridge.

60. The device of paragraph 55 or 56, wherein the lysed sample is enriched prior to functionalization.

61. The device of any one of paragraphs 55-60, wherein the lysed sample is fragmented prior to functionalization.

62. The device of any one of paragraphs 55-61, wherein the functionalization cartridge comprises a first chamber comprising reagents that covalently modify a moiety M⁰ of the one or more target molecules, or of one or more fragments thereof, to a modified moiety M¹.

63. The device of paragraph 62, wherein the reagents are non-enzymatic.

64. The device of paragraph 62 or 63, wherein the covalent modification is regiospecific.

65. The device of any one of paragraphs 62-64, wherein the portion of the one or more target molecules, or of the one or more fragments thereof, is a C-terminal carboxylate group or a C-terminal amino group.

66. The device of any one of paragraphs 62-65, wherein the reagents comprise buffers, salts, organic compounds, acids, and/or bases.

67. The device of any one of paragraphs 62-66, wherein the portion of the one or more target molecules, or of the one or more fragments thereof, is a C-terminal amino group, and the covalent modification is diazo transfer.

68. The device of paragraph 67, wherein moiety M⁰ is —NH₂ and moiety M¹ is —N₃.

69. The device of paragraph 66, wherein the reagents comprise imidazole-1-sulfonyl azide and a copper salt (e.g., copper sulfate), and a buffer having a pH of about 10⁻¹¹.

70. The device of any one of paragraphs 55-69, wherein the first chamber is connected via one or more microfluidic channels, and/or optionally a purification chamber, to a second chamber.

71. The device of paragraph 70, wherein the second chamber comprises reagents that covalently modify moiety M¹ to produce a functionalized peptide.

72. The device of paragraph 71, wherein the covalent modification is an electrocyclic click reaction.

73. The device of paragraph 71 or 72, wherein the reagents comprise a DBCO-labeled DNA-streptavidin conjugate and a buffer, optionally wherein the DBCO-labeled DNA-streptavidin conjugate is immobilized to the surface of the second chamber.

74. The device of paragraph 73, wherein the functionalized peptide is functionalized with a DBCO-labeled DNA-streptavidin conjugate.

75. The device of any one of paragraphs 70-72, comprising a purification chamber positioned between the first chamber and the second chamber, comprising a resin that promotes purification or enrichment of the modified target molecules, or fragments thereof.

76. The device of paragraph 75, wherein the resin is Sephadex resin, optionally G-10 Sephadex resin.

77. The device of any one of paragraphs 55-76, wherein the functionalization cartridge can be heated at an elevated temperature (e.g., 20-60° C.).

78. The device of any one of paragraphs 55-77, wherein the device is configured to heat the functionalization cartridge at an elevated temperature (e.g., 20-60° C.).

79. The device of any one of paragraphs 55-78, wherein the functionalization cartridge can be subjected to microwaves or sonication.

80. The device of any one of paragraphs 55-79, wherein the device is configured to subject the functionalization cartridge to microwaves or sonication.

81. The device of any preceding paragraph, wherein the device further comprises a peristaltic pump configured to transport one or more fluids into, within, or out of any one of cartridges received by the device.

82. The device of any preceding paragraph, wherein the device further comprises a peristaltic pump configured to transport one or more fluids within, or through any of the microfluidic channels of cartridges received by the device.

83. The device of any preceding paragraphs, wherein the device is configured to transport fluids with a fluid flow resolution of less than or equal to 1000 microliters, less than or equal to 100 microliters, less than or equal to 50 microliters, or less than or equal to 10 microliters.

84. The device of any preceding paragraph, wherein any one of the cartridges comprises a base layer having a surface comprising channels.

85. The device of paragraph 84, wherein the channels include the one or more microfluidic channels.

86. The device of paragraph 84 or 85, wherein at least a portion of at least some of the channels have a substantially triangularly-shaped cross-section having a single vertex at a base of the channel and having two other vertices at the surface of the base layer.

87. The device of any preceding paragraph, wherein, at least a portion of at least some of the channels of any one of the cartridges have a surface layer, comprising an elastomer, configured to substantially seal off a surface opening of the channel.

88. The device of paragraph 87, wherein the elastomer comprises silicone.

89. The device of any preceding paragraph, wherein, at least one portion of at least some of the channels have walls and a base comprising a substantially rigid material compatible with biological material.

90. The device of any preceding paragraph, wherein any one of the cartridges comprise one or more fluid reservoirs.

91. The device of any preceding paragraph, wherein at least some of the channels connect to a reservoir in a temperature zone.

92. The device of any preceding paragraph, wherein at least some of the channels connect to an electrophoresis gel.

93. The device of any preceding paragraph, wherein the device is configured to receive two or more cartridges at the same time.

94. The device of paragraph 93, wherein the device is configured to establish fluidic communication between two or more cartridges received by the device at the same time.

93. The device of any preceding paragraph, wherein the device is configured to receive two or more cartridges sequentially.

94. The device of any preceding paragraph, wherein the device further comprises a sequencing module.

95. The device of paragraph 94, wherein the device is configured to deliver the one or more target molecules to the sequencing module.

96. The device of paragraph 94 or 95, wherein the sequencing module performs nucleic acid sequencing.

97. The device of paragraph 96, wherein the nucleic acid sequencing comprises single-molecule real-time sequencing, sequencing by synthesis, sequencing by ligation, nanopore sequencing, and/or Sanger sequencing.

98. The device of paragraph 94 or 96, wherein the sequencing module performs protein sequencing.

99. The device of paragraph 98, wherein the protein sequencing comprises edman degradation or mass spectroscopy.

100. The device of paragraph 94 or 96, wherein the sequencing module performs single-molecule protein sequencing.

101. A device for preparing one or more target molecules, configured to perform step (iii) fragment the one or more target molecules; and one or more of the following steps selected from (i), (ii), and (iv),

wherein (i), (ii), and (iv) are defined as follows:
(i) lyse a biological sample comprising one or more target molecules;
(ii) enrich at least one of the one or more target molecules and/or at least one non-target molecule; and
(iv) functionalize a terminal moiety of the one or more target molecules.

102. The device of paragraph 101, wherein one or more of the steps selected from (i), (ii), (iii), and (iv) are performed in a cartridge.

103. The device of paragraph 101, wherein the one or more steps are performed in the same cartridge.

104. The device of paragraph 102 or 103, wherein the cartridge is a single-use cartridge or a multi-use cartridge.

105. The device of any one of paragraphs 102-104, wherein the cartridge comprises one or more microfluidic channels configured to contain and/or transport a fluid used in any one of the automated steps.

106. The device of any one of paragraphs 102-104, wherein the cartridge comprises one or more microfluidic channels configured to contain and/or transport the one or more target molecules between any one of the automated steps.

107. The device of any one of paragraphs 102-106, wherein the cartridge comprises resin for purification of the one or more target molecules between any one of the automated steps.

108. The device of paragraph 107, wherein the resin is Sephadex resin, optionally G-10 Sephadex resin.

109. The device of any one of paragraphs 101-108, wherein the biological sample is a single cell, mammalian cell tissue, animal sample, fungal sample, or plant sample.

110. The device of any one of paragraphs 101-109, wherein the biological sample is a blood sample, saliva sample, sputum sample, fecal sample, urine sample, buccal swab sample, amniotic sample, seminal sample, synovial sample, spinal sample, or pleural fluid sample.

111. The device of any one of paragraphs 101-110, wherein the one or more target molecules are nucleic acids.

112. The device of any one of paragraphs 101-110, wherein the one or more target molecules are proteins.

113. The device of any one of paragraphs 101-112, wherein step (iii) is performed in a fragmentation cartridge or a fragmentation section of a cartridge.

114. The device of paragraph 113, wherein the fragmentation cartridge or the fragmentation section of the cartridge comprises non-enzymatic reagents that digest or fragment the sample and/or the one or more target molecules.

115. The device of paragraph 114, wherein the non-enzymatic reagents that digest or fragment the sample and/or the one or more target molecules comprise detergents, acids, and/or bases.

116. The device of paragraph 114 or 115, wherein the non-enzymatic reagents that digest or fragment the sample and/or the one or more target molecules comprise cyanogen bromide, hydroxylamine, iodosobenzoic acid, dimethyl sulfoxide, hydrochloric acid, BNPS-skatole [2-(2-nitrophenylsulfenyl)-3-methylindole], and/or 2-nitro-5-thiocyanobenzoic acid.

117. The device of any one of paragraphs 113-116, wherein the fragmentation cartridge or the fragmentation section of the cartridge comprises one or more enzymatic reagents that digest or fragment at least one of the one or more target molecules.

118. The device of paragraph 117, wherein the one or more enzymatic reagents comprise one or more proteases.

119. The device of paragraph 118, wherein the one or more proteases are selected from the group consisting of: trypsin, chymotrypsin, LysC, LysN, AspN, GluC and ArgC.

120. The device of paragraph 118, wherein the one or more enzymatic reagents comprise one or more endonucleases or exonucleases.

121. The device of any one of paragraphs 113-120, wherein the fragmentation cartridge or the fragmentation section of the cartridge can be heated at an elevated temperature (e.g., 20-60° C.).

122. The device of any one of paragraphs 113-121, wherein the device is configured to heat the fragmentation cartridge or the fragmentation section of the cartridge at an elevated temperature (e.g., 20-60° C.).

123. The device of any one of paragraphs 113-122, wherein the device is configured to subject the fragmentation cartridge or the fragmentation section of the cartridge to microwaves or sonication.

124. The device of any one of paragraphs 102-123, wherein step (i) is performed in a lysis cartridge or a lysis section of a cartridge.

125. The device of paragraph 124, wherein the fragmentation cartridge is positioned to receive the lysed sample from the lysis cartridge or the fragmentation section of the cartridge is positioned to receive the lysed sample from the lysis section of the cartridge.

126. The device of paragraph 124 or 125, wherein the lysis cartridge and the fragmentation cartridge or lysis section of the cartridge and the fragmentation section of the cartridge are connected by one or more microfluidic channels.

127. The device of paragraph 124 or 126, wherein the lysis cartridge or the lysis section of the cartridge comprises reagents that lyse the sample but does not degrade or fragment the one or more target molecules.

128. The device of paragraph 124 or 127, wherein the lysis cartridge or the lysis section of the cartridge comprises reagents that promote the one or more target molecules to be at least partially isolated or purified from non-target molecules of the sample.

129. The device of paragraph 127 or 128, wherein the reagents comprise detergents, acids, and/or bases.

130. The device of any one of paragraphs 127-129, wherein the reagents comprise a lysis buffer.

131. The device of paragraph 130, wherein the lysis buffer is selected from the group consisting of: RIPA buffer, GCl (Guanidine-HCl) buffer, and GlyNP40 buffer.

132. The device of any one of paragraphs 124-131, wherein the one or more microfluidic channels in the lysis cartridge or the lysis section of the cartridge promote shearing of cells and/or tissues (e.g., shear flow of cells and/or tissues).

133. The device of any one of paragraphs 124-132, wherein the lysis cartridge or the lysis section of the cartridge comprises a needle passage that promotes mechanical shearing of cells and/or tissues.

134. The device of paragraph 133, wherein the needle passage has an internal diameter of 0.1 to 1 mm.

135. The device of any one of paragraphs 124-134, wherein the one or more microfluidic channels in the lysis cartridge or the lysis section of the cartridge comprise a post array.

136. The device of any one of paragraphs 124-135, wherein the lysis cartridge or the lysis section of the cartridge is configured to be heated at an elevated temperature (e.g., 20-60° C.).

137. The device of any one of paragraphs 124-136, wherein the device is configured to heat the lysis cartridge or the lysis section of the cartridge at an elevated temperature (e.g., 20-60° C.).

138. The device of any one of paragraphs 124-137, wherein the device is configured to subject the lysis cartridge or the lysis section of the cartridge to microwaves or sonication.

139. The device of any one of paragraphs 102-138, wherein step (ii) is performed in a enrichment cartridge or a enrichment section of a cartridge.

140. The device of paragraph 139, wherein the enrichment cartridge is positioned to receive the lysed sample from the lysis cartridge or the enrichment section of the cartridge is positioned to receive the lysed sample from the lysis section of the cartridge.

141. The device of paragraph 139 or 140, wherein the lysis cartridge and the enrichment cartridge or the lysis section of the cartridge and the enrichment section of the cartridge are connected by one or more microfluidic channels.

142. The device of paragraph 139, wherein the fragmentation cartridge is positioned to receive the enriched sample from the enrichment cartridge or the fragmentation section of the cartridge is positioned to receive the enriched sample from the enrichment section of the cartridge.

143. The device of paragraph 142, wherein the enrichment cartridge and the fragmentation cartridge or the enrichment section of the cartridge and the fragmentation section of the cartridge are connected by one or more microfluidic channels.

144. The device of any one of paragraphs 139-143, wherein the enrichment cartridge or the enrichment section of the cartridge comprises one or more affinity matrices.

145. The device of paragraph 144, wherein the one or more affinity matrices are in microfluidic channels of the enrichment cartridge or the enrichment section of the cartridge.

146. The device of paragraph 144, wherein the one or more target molecules are nucleic acids, wherein the immobilized capture probe is an oligonucleotide capture probe, and wherein the oligonucleotide capture probe comprises a sequence that is at least partially complementary to at least one of the one or more target molecules.

147. The device of paragraph 146, wherein the oligonucleotide capture probe comprises a sequence that is at least 80%, 90% 95%, or 100% complementary to the target molecule.

148. The device of any one of paragraphs 144-147, wherein the device produces nucleic acids with an average read-length that is longer than an average read-length produced using control methods.

149. The device of paragraph 144, wherein the one or more target molecules are proteins, and wherein the immobilized capture probe is a protein capture probe that binds to at least one of the one or more target molecules.

150. The device of paragraph 149, wherein the protein capture probe is an aptamer or an antibody.

151. The device of paragraph 149 or 150, wherein the protein capture probe binds to the target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M.

152. The device of paragraph 144, wherein the one or more target molecules are nucleic acids, wherein the immobilized capture probe is an oligonucleotide capture probe, and wherein the oligonucleotide capture probe comprises a sequence that is at least partially complementary to at least one non-target molecule.

153. The device of paragraph 152, wherein the oligonucleotide capture probe comprises a sequence that is at least 80%, 90% 95%, or 100% complementary to the non-target molecule.

154. The device of paragraph 152 or 153, wherein the oligonucleotide capture probe is not complementary to the one or more target molecules.

155. The device of paragraph 144, wherein the one or more target molecules are proteins, and wherein the immobilized capture probe is a protein capture probe that binds to at least one non-target molecule.

156. The device of paragraph 155, wherein the protein capture probe is an aptamer or an antibody.

157. The device of paragraph 155 or 156, wherein the protein capture probe binds to the non-target protein with a binding affinity of 10⁻⁹ to 10⁻⁸ M, 10⁻⁸ to 10⁻⁷ M, 10⁻⁷ to 10⁻⁶ M, 10-6 to 10⁻⁵ M, 10⁻⁵ to 10⁻⁴ M, 10⁻⁴ to 10⁻³ M, or 10⁻³ to 10⁻² M.

158. The device of any one of paragraphs 155-157, wherein the protein capture probe does not bind to the one or more target molecules.

159. The device of any one of paragraphs 152-158, wherein the enrichment cartridge or the enrichment section of the cartridge is configured to deplete the sample of non-target molecules.

160. The device of any one of paragraphs 113-153, wherein step (iv) is performed in a functionalization cartridge or a functionalization section of a cartridge.

161. The device of paragraph 160, wherein the lysis cartridge and the functionalization cartridge or the lysis section of the cartridge and the functionalization section of the cartridge are connected by one or more microfluidic channels.

162. The device of paragraph 160, wherein the enrichment cartridge and the functionalization cartridge or the enrichment section of the cartridge and the functionalization section of the cartridge are connected by one or more microfluidic channels.

163. The device of paragraph 160, wherein the fragmentation cartridge and the functionalization cartridge or the fragmentation section of the cartridge and the functionalization section of the cartridge are connected by one or more microfluidic channels.

164. The device of paragraph 163, wherein the functionalization cartridge is positioned to receive the fragmented sample from the fragmentation cartridge.

165. The device of paragraph 160 or 161, wherein the lysed sample is enriched prior to functionalization.

166. The device of any one of paragraphs 160-165, wherein the lysed sample is fragmented prior to functionalization.

167. The device of any one of paragraphs 160-166, wherein the functionalization cartridge or the functionalization section of the cartridge comprises a first chamber comprising reagents that covalently modify a moiety M⁰ of the one or more target molecules, or of one or more fragments thereof, to a modified moiety M¹.

168. The device of paragraph 167, wherein the reagents are non-enzymatic.

169. The device of paragraph 167 or 168, wherein the covalent modification is regiospecific.

170. The device of any one of paragraphs 167-169, wherein the portion of the one or more target molecules, or of the one or more fragments thereof, is a C-terminal carboxylate group or a C-terminal amino group.

171. The device of any one of paragraphs 167-170, wherein the reagents comprise buffers, salts, organic compounds, acids, and/or bases.

172. The device of any one of paragraphs 167-171, wherein the portion of the one or more target molecules, or of the one or more fragments thereof, is a C-terminal amino group, and the covalent modification is diazo transfer.

173. The device of paragraph 172, wherein moiety M⁰ is —NH₂ and moiety M¹ is —N₃.

174. The device of paragraph 171, wherein the reagents comprise imidazole-1-sulfonyl azide and a copper salt (e.g., copper sulfate), and a buffer having a pH of about 10-11.

175. The device of any one of paragraphs 160-174, wherein the first chamber is connected via one or more microfluidic channels, and/or optionally a purification chamber, to a second chamber.

176. The device of paragraph 175, wherein the second chamber comprises reagents that covalently modify moiety M¹ to produce a functionalized peptide.

177. The device of paragraph 176, wherein the covalent modification is an electrocyclic click reaction.

178. The device of paragraph 176 or 177, wherein the reagents comprise a DBCO-labeled DNA-streptavidin conjugate and a buffer, optionally wherein the DBCO-labeled DNA-streptavidin conjugate is immobilized to the surface of the second chamber.

179. The device of paragraph 178, wherein the functionalized peptide is functionalized with a DBCO-labeled DNA-streptavidin conjugate.

180. The device of any one of paragraphs 175-177, comprising a purification chamber positioned between the first chamber and the second chamber, comprising a resin that promotes purification or enrichment of the modified target molecules, or fragments thereof.

181. The device of paragraph 180, wherein the resin is Sephadex resin, optionally G-10 Sephadex resin.

182. The device of any one of paragraphs 160-181, wherein the functionalization cartridge or the functionalization section of the cartridge can be heated at an elevated temperature (e.g., 20-60° C.).

183. The device of any one of paragraphs 160-182, wherein the device is configured to heat the functionalization cartridge or the functionalization section of the cartridge at an elevated temperature (e.g., 20-60° C.).

184. The device of any one of paragraphs 160-183, wherein the functionalization cartridge or the functionalization section of the cartridge can be subjected to microwaves or sonication.

185. The device of any one of paragraphs 160-184, wherein the device is configured to subject the functionalization cartridge or the functionalization section of the cartridge to microwaves or sonication.

186. The device of any preceding paragraph, wherein the device further comprises a peristaltic pump configured to transport one or more fluids into, within, or out of any one of cartridges received by the device.

187. The device of any preceding paragraph, wherein the device further comprises a peristaltic pump configured to transport one or more fluids within, or through any of the microfluidic channels of cartridges received by the device.

188. The device of any preceding paragraphs, wherein the device is configured to transport fluids with a fluid flow resolution of less than or equal to 1000 microliters, less than or equal to 100 microliters, less than or equal to 50 microliters, or less than or equal to 10 microliters.

189. The device of any preceding paragraph, wherein any one of the cartridges comprises a base layer having a surface comprising channels.

190. The device of paragraph 189, wherein the channels include the one or more microfluidic channels.

191. The device of paragraph 189 or 190, wherein at least a portion of at least some of the channels have a substantially triangularly-shaped cross-section having a single vertex at a base of the channel and having two other vertices at the surface of the base layer.

192. The device of any preceding paragraph, wherein, at least a portion of at least some of the channels of any one of the cartridges have a surface layer, comprising an elastomer, configured to substantially seal off a surface opening of the channel.

193. The device of paragraph 192, wherein the elastomer comprises silicone.

194. The device of any preceding paragraph, wherein, at least one portion of at least some of the channels have walls and a base comprising a substantially rigid material compatible with biological material.

195. The device of any preceding paragraph, wherein any one of the cartridges comprise one or more fluid reservoirs.

196. The device of any preceding paragraph, wherein at least some of the channels connect to a reservoir in a temperature zone.

197. The device of any preceding paragraph, wherein at least some of the channels connect to an electrophoresis gel.

198. The device of any preceding paragraph, wherein the device is configured to receive two or more cartridges at the same time.

199. The device of paragraph 198, wherein the device is configured to establish fluidic communication between two or more cartridges received by the device at the same time.

200. The device of any preceding paragraph, wherein the device is configured to receive two or more cartridges sequentially.

201. The device of any preceding paragraph, wherein the device further comprises a sequencing module.

202. The device of paragraph 201, wherein the device is configured to deliver the one or more target molecules to the sequencing module.

203. The device of paragraph 201 or 202, wherein the sequencing module performs nucleic acid sequencing.

204. The device of paragraph 203, wherein the nucleic acid sequencing comprises single-molecule real-time sequencing, sequencing by synthesis, sequencing by ligation, nanopore sequencing, and/or Sanger sequencing.

205. The device of paragraph 201 or 203, wherein the sequencing module performs protein sequencing.

206. The device of paragraph 205, wherein the protein sequencing comprises edman degradation or mass spectroscopy.

207. The device of paragraph 201 or 203, wherein the sequencing module performs single-molecule protein sequencing.

208. A method for preparing one or more target molecules, comprising step (iii) fragment the one or more target molecules; and one or more of the following steps selected from (i), (ii), and (iv),

wherein (i), (ii), and (iv) are defined as follows:
(i) lyse a biological sample comprising one or more target molecules
(ii) enrich at least one of the one or more target molecules and/or at least non-target molecule; and
(iv) functionalize a terminal moiety of the one or more fragmented target molecules;

wherein step (iii) is performed in an automated sample preparation device.

209. The method of paragraph 208, wherein the biological sample is a single cell, mammalian cell tissue, animal sample, fungal sample, or plant sample.

210. The method of paragraph 208, wherein the biological sample is a blood sample, saliva sample, sputum sample, fecal sample, urine sample, buccal swab sample, amniotic sample, seminal sample, synovial sample, spinal sample, or pleural fluid sample.

211. The method of any one of paragraphs 208-210, wherein the one or more target molecules are nucleic acids.

212. The method of any one of paragraphs 208-210, wherein the one or more target molecules are proteins.

213. The method of paragraph 208, wherein two steps are performed in an automated sample preparation device.

214. The method of paragraph 208, wherein three steps are performed in an automated sample preparation device.

215. The method of paragraph 208, wherein four steps are performed in an automated sample preparation device.

216. The method of any one of paragraphs 208-215, wherein step (iii) is performed using a fragmentation cartridge.

217. The method of paragraph 216, wherein step (iii) is performed using more than one fragmentation cartridge (e.g., two or three fragmentation cartridges).

218. The method of any one of paragraphs 216-217, wherein the fragmentation cartridge comprises non-enzymatic reagents that digest or fragment the sample and/or the one or more target molecules.

219. The method of paragraph 218, wherein the non-enzymatic reagents that digest or fragment the sample and/or the one or more target molecules comprise detergents, acids, and/or bases.

220. The method of paragraph 218 or 219, wherein the non-enzymatic reagents that digest or fragment the sample and/or the one or more target molecules comprise cyanogen bromide, hydroxylamine, iodosobenzoic acid, dimethyl sulfoxide, hydrochloric acid, BNPS-skatole [2-(2-nitrophenylsulfenyl)-3-methylindole], and/or 2-nitro-5-thiocyanobenzoic acid.

221. The method of any one of paragraphs 217-220, wherein the fragmentation cartridge comprises one or more enzymatic reagents that digest or fragment at least one of the one or more target molecules.

222. The method of paragraph 221, wherein the one or more enzymatic reagents comprise one or more proteases.

223. The method of paragraph 222, wherein the one or more proteases are selected from the group consisting of: trypsin, chymotrypsin, LysC, LysN, AspN, GluC and ArgC.

224. The method of paragraph 222, wherein the one or more enzymatic reagents comprise one or more endonucleases or exonucleases.

225 The method of any one of paragraphs 217-224, wherein the fragmentation cartridge can be heated at an elevated temperature (e.g., 20-60° C.).

226. The method of any one of paragraphs 217-225, wherein the method is configured to heat the fragmentation cartridge at an elevated temperature (e.g., 20-60° C.).

227. The method of any one of paragraphs 217-226, wherein the method is configured to subject the fragmentation cartridge to microwaves or sonication.

228. The method of any one of paragraphs 208-227, wherein step (i) is performed using a lysis cartridge.

229. The method of paragraph 228, wherein the lysis cartridge comprises one or more microfluidic channels configured to contain and/or transport fluid(s) and/or reagent(s).

230. The method of any one of paragraphs 228 or 229, wherein the lysis cartridge comprises reagents that lyse the sample but does not degrade or fragment the one or more target molecules.

231. The method of any one of paragraphs 228-230, wherein the lysis cartridge comprises reagents that promote the one or more target molecules to be at least partially isolated or purified from non-target molecules of the sample.

232. The method of any one of paragraphs 230 or 231, wherein the reagents comprise detergents, acids, and/or bases.

233. The method of any one of paragraphs 230-232, wherein the reagents comprise a lysis buffer.

234. The method of paragraph 233, wherein the lysis buffer is selected from the group consisting of: RIPA buffer, GCl (Guanidine-HCl) buffer, and GlyNP40 buffer.

235. The method of any one of paragraphs 228-234, wherein the one or more microfluidic channels in the lysis cartridge promote shearing of cells and/or tissues (e.g., shear flow of cells and/or tissues).

236. The method of any one of paragraphs 228-235, wherein the lysis cartridge comprises a needle passage that promotes mechanical shearing of cells and/or tissues.

237. The method of paragraph 236, wherein the needle passage has an internal diameter of 0.1 to 1 mm.

238. The method of any one of paragraphs 228-237, wherein the one or more microfluidic channels in the lysis cartridge comprise a post array.

239. The method of any one of paragraphs 228-238, wherein the lysis cartridge is configured to be heated at an elevated temperature (e.g., 20-60° C.).

240. The method of any one of paragraphs 228-239, wherein the device is configured to heat the lysis cartridge at an elevated temperature (e.g., 20-60° C.).

241. The method of any one of paragraphs 228-240, wherein the device is configured to subject the lysis cartridge to microwaves or sonication.

242. The method of any one of paragraphs 208-241, wherein step (ii) is performed in an automated sample preparation device.

243. The method of paragraph 242, wherein step (ii) is performed using an enrichment cartridge.

244. The method of paragraph 243, wherein the enrichment cartridge comprises one or more affinity matrices.

245. The method of paragraph 244, wherein the one or more affinity matrices are in microfluidic channels of the enrichment cartridge.

246. The method of paragraph 244, wherein the one or more target molecules are nucleic acids, wherein the immobilized capture probe is an oligonucleotide capture probe, and wherein the oligonucleotide capture probe comprises a sequence that is at least partially complementary to at least one of the one or more target molecules.

247. The method of paragraph 246, wherein the oligonucleotide capture probe comprises a sequence that is at least 80%, 90% 95%, or 100% complementary to the target molecule.

248. The method of paragraph 244, wherein the one or more target molecules are proteins, and wherein the immobilized capture probe is a protein capture probe that binds to at least one of the one or more target molecules.

249. The method of paragraph 248, wherein the protein capture probe is an aptamer or an antibody.

250. The method of paragraph 248 or 249, wherein the protein capture probe binds to the target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M.

251. The method of paragraph 244, wherein the one or more target molecules are nucleic acids, wherein the immobilized capture probe is an oligonucleotide capture probe, and wherein the oligonucleotide capture probe comprises a sequence that is at least partially complementary to at least one non-target molecule.

252. The method of paragraph 251, wherein the oligonucleotide capture probe comprises a sequence that is at least 80%, 90% 95%, or 100% complementary to the non-target molecule.

253. The method of paragraph 251 or 252, wherein the oligonucleotide capture probe is not complementary to the one or more target molecules.

254. The method of paragraph 244, wherein the one or more target molecules are proteins, and wherein the immobilized capture probe is a protein capture probe that binds to at least one non-target molecule.

255. The method of paragraph 254, wherein the protein capture probe is an aptamer or an antibody.

256. The method of paragraph 254 or 255, wherein the protein capture probe binds to the non-target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M.

257. The device of any one of paragraphs 254-256, wherein the protein capture probe does not bind to the one or more target molecules.

258. The device of any one of paragraphs 251-257, wherein the enrichment cartridge is configured to deplete the sample of non-target molecules.

259. The method of any one of claims 208-258, wherein step (iv) is performed in an automated sample preparation device.

260. The method of claim 259, wherein step (iv) is performed using a functionalization cartridge.

261. The method of claim 260, wherein the functionalization cartridge comprises a first chamber comprising reagents that covalently modify a moiety M⁰ of the one or more target molecules, or of one or more fragments thereof, to a modified moiety M¹.

262. The method of claim 261, wherein the reagents are non-enzymatic.

263. The method of claim 261 or 262, wherein the covalent modification is regiospecific.

264. The method of any one of claims 261-263, wherein the portion of the one or more target molecules, or of the one or more fragments thereof, is a C-terminal carboxylate group or a C-terminal amino group.

265. The method of any one of claims 261-264, wherein the reagents comprise buffers, salts, organic compounds, acids, and/or bases.

266. The method of any one of claims 261-265, wherein the portion of the one or more target molecules, or of the one or more fragments thereof, is a C-terminal amino group, and the covalent modification is diazo transfer.

267. The method of claim 266, wherein moiety M⁰ is —NH₂ and moiety M¹ is —N₃.

268. The method of claim 265, wherein the reagents comprise imidazole-1-sulfonyl azide and a copper salt (e.g., copper sulfate), and a buffer having a pH of about 10-11.

269. The method of any one of claims 260-268, wherein the first chamber is connected via one or more microfluidic channels, and/or optionally a purification chamber, to a second chamber.

270. The method of claim 269, wherein the second chamber comprises reagents that covalently modify moiety M¹ to produce a functionalized peptide.

271. The method of claim 270, wherein the covalent modification is an electrocyclic click reaction.

272. The method of claim 270 or 271, wherein the reagents comprise a DBCO-labeled DNA-streptavidin conjugate and a buffer, optionally wherein the DBCO-labeled DNA-streptavidin conjugate is immobilized to the surface of the second chamber.

273. The method of claim 272, wherein the functionalized peptide is functionalized with a DBCO-labeled DNA-streptavidin conjugate.

274. The method of any one of claims 269-271, comprising a purification chamber positioned between the first chamber and the second chamber, comprising a resin that promotes purification or enrichment of the modified target molecules, or fragments thereof.

275. The method of claim 274, wherein the resin is Sephadex resin, optionally G-10 Sephadex resin.

276. The method of any one of claims 260-275, wherein the functionalization cartridge can be heated at an elevated temperature (e.g., 20-60° C.).

277. The method of any one of claims 260-276, wherein the method is configured to heat the functionalization cartridge at an elevated temperature (e.g., 20-60° C.).

278. The method of any one of claims 260-277, wherein the functionalization cartridge can be subjected to microwaves or sonication.

279. The method of any one of claims 260-278, wherein the method is configured to subject the functionalization cartridge to microwaves or sonication.

280. The method of any one of claims 208-215, wherein two or more of steps (i), (ii), and (iii) are performed in a single cartridge.

281. A cartridge for preparing one or more target molecules, configured to perform step (iii) fragment the one or more target molecules; and one or more of the following steps selected from (i), (ii), and (iv),

wherein (i), (ii), and (iv) are defined as follows:

(i) lyse a biological sample comprising one or more target molecules

(ii) enrich at least one of the one or more target molecules and/or at least one non-target molecule; and

(iv) functionalize a terminal moiety of the one or more target molecules.

282. The cartridge of claim 281, wherein the cartridge is a single-use cartridge or a multi-use cartridge.

283. The cartridge of claim 281 or 282, wherein the cartridge comprises one or more microfluidic channels configured to contain and/or transport a fluid used in any one of the automated steps.

284. The cartridge of claim 281 or 282, wherein the cartridge comprises one or more microfluidic channels configured to contain and/or transport the one or more target molecules between any one of the automated steps.

285. The cartridge of any one of claims 281-284, wherein the cartridge comprises resin for purification of the one or more target molecules between any one of the automated steps.

286. The cartridge of claim 285, wherein the resin is Sephadex resin, optionally G-10 Sephadex resin.

### FURTHER ASPECTS OF THE INVENTION

Aspects of the exemplary embodiments and examples described above may be combined in various combinations and subcombinations to yield further embodiments of the invention. To the extent that aspects of the exemplary embodiments and examples described above are not mutually exclusive, it is intended that all such combinations and subcombinations are within the scope of the present invention. It will be apparent to those of skill in the art that embodiments of the present invention include a number of aspects. Accordingly, the scope of the claims should not be limited by the preferred embodiments set forth in the description and examples, but should be given the broadest interpretation consistent with the description as a whole.

What is claimed is:

1. A device for preparing a biological sample for sequencing, wherein the device comprises an automated module configured to receive (iii) a fragmentation cartridge comprising one or more microfluidic channels and configured to digest or fragment at least one of the one or more target molecules to produce a fragmented sample; and one or more of the cartridges selected from (i) a lysis cartridge (ii) an enrichment cartridge, and (iv) a functionalization cartridge; wherein (i), (ii), and (iv) are defined as follows:
(i) a lysis cartridge comprises one or more microfluidic channels and configured to intake a biological sample comprising one or more target molecules and produce a lysed sample;
(ii) an enrichment cartridge comprises one or more microfluidic channels and is configured to enrich at least one of the one or more target molecules to produce an enriched sample; and
(iv) a functionalization cartridge comprises one or more microfluidic channels and is configured to functionalize a terminal moiety of at least one of the one or more target molecules to produce a functionalized sample.

2. The device of claim 1, wherein the biological sample is a single cell, mammalian cell tissue, animal sample, fungal sample, plant sample, blood sample, saliva sample, sputum sample, fecal sample, urine sample, buccal swab sample, amniotic sample, seminal sample, synovial sample, spinal sample, or pleural fluid sample.

3. The device of claim 1, wherein the one or more target molecules are nucleic acids or proteins.

4. The device of claim 1, wherein the one or more microfluidic channels are configured to contain and/or transport fluid(s) and/or reagent(s).

5. The device of claim 1, wherein the fragmentation cartridge comprises non-enzymatic reagents that digest or fragment the sample and/or the one or more target molecules.

6. The device of claim 5, wherein the non-enzymatic reagents that digest or fragment the sample and/or the one or more target molecules comprise detergents, acids, and/or bases.

7. The device of claim 5, wherein the non-enzymatic reagents that digest or fragment the sample and/or the one or more target molecules comprise cyanogen bromide, hydroxylamine, iodosobenzoic acid, dimethyl sulfoxide, hydrochloric acid, BNPS-skatole [2-(2-nitrophenylsulfenyl)-3-methylindole], and/or 2-nitro-5-thiocyanobenzoic acid.

8. The device of claim 1, wherein the fragmentation cartridge comprises one or more enzymatic reagents that digest or fragment at least one of the one or more target molecules.

9. The device of claim 8, wherein the one or more enzymatic reagents comprise one or more proteases, optionally wherein the one or more proteases are selected from the group consisting of: trypsin, chymotrypsin, LysC, LysN, AspN, GluC and ArgC.

10. The device of claim 8, wherein the one or more enzymatic reagents comprise one or more endonucleases or exonucleases.

11. The device of claim 1, wherein the fragmentation cartridge can be heated at an elevated temperature (e.g., 20-60° C.).

12. The device of claim 1, wherein the device is configured to heat the fragmentation cartridge at an elevated temperature (e.g., 20-60° C.).

13. The device of claim 1, wherein the module is further configured to receive a lysis cartridge, optionally wherein the lysis cartridge and the fragmentation cartridge are connected by one or more microfluidic channels.

14. The device of claim 1, wherein the fragmentation cartridge is positioned to receive the lysed sample from the lysis cartridge.

15. The device of claim 13, wherein the device is configured such that the lysed sample is enriched prior to fragmentation.

16. A device for preparing one or more target molecules, configured to perform step (iii) fragment the one or more target molecules; and one or more of the following steps selected from (i), (ii), and (iv),
wherein (i), (ii), and (iv) are defined as follows:
(i) lyse a biological sample comprising one or more target molecules;
(ii) enrich at least one of the one or more target molecules and/or at least one non-target molecule; and
(iv) functionalize a terminal moiety of the one or more target molecules.

17. The device of claim 16, wherein one or more of the steps selected from (i), (ii), (iii), and (iv) are performed in a cartridge.

18. The device of claim 16, wherein the one or more steps are performed in the same cartridge.

19. A method for preparing one or more target molecules, comprising step (iii) fragment the one or more target molecules; and one or more of the following steps selected from (i), (ii), and (iv),
wherein (i), (ii), and (iv) are defined as follows:
(i) lyse a biological sample comprising one or more target molecules
(ii) enrich at least one of the one or more target molecules and/or at least non-target molecule; and
(iv) functionalize a terminal moiety of the one or more fragmented target molecules;
wherein step (iii) is performed in an automated sample preparation device.

20. A cartridge for preparing one or more target molecules, configured to perform step (iii) fragment the one or more target molecules; and one or more of the following steps selected from (i), (ii), and (iv),
wherein (i), (ii), and (iv) are defined as follows:
(i) lyse a biological sample comprising one or more target molecules
(ii) enrich at least one of the one or more target molecules and/or at least one non-target molecule; and
(iv) functionalize a terminal moiety of the one or more target molecules.
